SEQUENCE LISTING (69 sequences)

SEQ ID NO: 1 - DNA, 4856 bp, Artificial Sequence
  Synthetic vector
gatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatccaatgtttaaacaagcttgttccagctatgagaactttttatcaagctcttgtacaatctggtttaaatggtattaaggtttcatcacctcattcgttgggtatactcttacgatccaatccaccaagtgctgcaagatttagacctggatgggatgttggtattcttgctccaatgcttcagtttctacgcgaaactaaaggaccgttcatggtaaacccatatccatactttggttacaacccgaaacaagaagactttcttcttttcaggaaaaacaaaggtgtttatgacaggttttcaaaaagatggtatacgaatagtttcgatatgttgttggatgctgtgtatatgtcaatggtaaggttgaagtatccagatgtggagattgtagctgctgaaacagggtggccttctcaaggtgaatcatatgagcctcaatgtacagtggaaaatgcagcttcgtataatgtatgctatacgaaatcgcaagatattataaaaggagaggtttatatagatgtaataataataataacgtgtagtggaaggtaaaaaaggtagaggggcagaggaaaagcttatcgaaaatcgaagaaaaacccaaaggaggattcaaagaaagaagaaagatgacgacaaagaggcaaagttttgaaagtaaaagggtccgttaaaaagttgttttttcttttcttcttctttagtgaagtgagtagtacttgtagtatcaaacgttcaattgaaatcatagttaaaagttaatcatgagagcttagctaactgttgggacacttggactgaaattttcttacttacacttttatatttttctgttctttctctaacatttgttctcattgacaattcaccacacatatgagtggttcgctagttcgatatggccatgagttgagattatatatgctttggccaagtggatattatattgcaattaatctactatcagatgtggcaaccttggatttgctgaaaacggaaaatctgcattgggttggatttcttaaaagtaatgtatctaaaaaaatatagtcatgtttaacggtgctgaattttgccaactggacaagaatgcaaatgttacacattgtcatccaccaattaggaaatagatagtgatattcaaggataaggacttagggtctttcgagtcatttaaataaacttgttggaagatccatgaaactcatcaactcttctttctgtgtaatagctgcattcaagagtttttcagttactagtttttagaattgagttttcactaattatcgggttgtttgatggccttgtaaatttggctattgcaaattatggtaatcatatatgaaactttgtttggtcttcaataattttgaatggccataaaatttaaaatcctctacgtcgacaccagaagttcaaaacaagcttgttccagctatgagaactttttatcaagctcttgtacaatctggtttaaatggtattaaggtttcatcacctcattcgttgggtatactcttacgatccaatccaccaagtgctgcaagatttagacctggatgggatgttggtattcttgctccaatgcttcagtttctacgcgaaactaaaggaccgttcatggtaaacccatatccatactttggttacaacccgaaacaagaagactttcttcttttcaggaaaaacaaaggtgtttatgacaggttttcaaaaagatggtatacgaatagtttcgatatgttgttggatgctgtgtatatgtcaatggtaaggttgaagtatccagatgtggagattgtagctgctgaaacagggtggccttctcaaggtgaatcatatgagcctcaatgtacagtggaaaatgcagcttcgtataatgtatgctatacgaaatcgcaagatattataaaaggagaggtttatatagatgtaataataataataacgtgtagtggaaggtaaaaaaggtagaggggcagaggaaaagcttatcgaaaatcgaagaaaaacccaaaggaggattcaaagaaagaagaaagatgacgacaaagaggcaaagttttgaaagtaaaagggtccgttaaaaagttgttttttcttttcttcttctttagtgaagtgagtagttgttaacattggatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcg

SEQ ID NO: 2 - DNA, 2272 bp, Artificial Sequence
  Synthetic vector
cttcgtataatgtatgctatacgaaatcgcaagatattataaaaggagaggtttatatagatgtaataataataataacgtgtagtggaaggtaaaaaaggtagaggggcagaggaaaagcttatcgaaaatcgaagaaaaacccaaaggaggattcaaagaaagaagaaagatgacgacaaagaggcaaagttttgaaagtaaaagggtccgttaaaaagttgttttttcttttcttcttctttagtgaagtgagtagtacttgtagtatcaaacgttcaattgaaatcatagttaaaagttaatcatgagagcttagctaactgttgggacacttggactgaaattttcttacttacacttttatatttttctgttctttctctaacatttgttctcattgacaattcaccacacatatgagtggttcgctagttcgatatggccatgagttgagattatatatgctttggccaagtggatattatattgcaattaatctactatcagatgtggcaaccttggatttgctgaaaacggaaaatctgcattgggttggatttcttaaaagtaatgtatctaaaaaaatatagtcatgtttaacggtgctgaattttgccaactggacaagaatgcaaatgttacacattgtcatccaccaattaggaaatagatagtgatattcaaggataaggacttagggtctttcgagtcatttaaataaacttgttggaagatccatgaaactcatcaactcttctttctgtgtaatagctgcattcaagagtttttcagttactagatttgacttattcaattcatcgcgtgatatatcatgaatacttctgtttttacgtcgtcgggggtactgaggaaaggagcatgggctgaagaagaagatattctcttaagaaaatgtattgagaagtacggggaaggaaagtggcatcaagttcctgttagagccggtttaaatagatgcaggaagagctgcaggctaaggtggttgaattatctgagaccacatataaagagaggtgacttttgtccggaggaagtggaccttattcagaggcttcataagcttctcggcaacaggtggtcacttattgccgggagacttccgggaagaacggcaaacgatgtgaaaaactactggaatactcaccttctaaggaggtcaaactttgctcctcctccccagcaacacgaaaggaaatgtactaaagaaattaggaccatggccaagaatgccataataagacctcaacctcggaatctctcaaaattagcaaagaataacgtctcaaaccacagtactaaacacaaggatgaatatagcaaacagaaaatgttcatcgagaagccaacaacggccgaagtcgtgtcgagagataacaacgttgaatggtggacgaatttattactggataactgcaacggatttgaaaaggcagcacctgaaagctcttcaacatttaagaacatagaaagtttgttaaacgaagaactattatcagcatcgataaatggtggaaccaactatcccattcaagaaactggagacatgggttggagtgacttttgtattgattctgacccctgggaattgctactccaatgaattactcagatatatgattggtactgttatcttgtactttttagaattgagttttcactaattatcgggttgtttgatggccttgtaaatttggctattgcaaattatggtaatcatatatgaaactttgtttggtcttcaataattttgaatggccataaaatttaaaatcctctacgtcgacaccagaagttcaaaacaagcttgttccagctatgagaactttttatcaagctcttgtacaatctggtttaaatggtattaaggtttcatcacctcattcgttgggtatactcttacgatccaatccaccaagtgctgcaagatttagacctggatgggatgttggtattcttgctccaatgcttcagtttctacgcgaaactaaaggaccgttcatggtaaacccatatccatactttggttacaacccgaaacaagaagactttcttcttttcaggaaaaacaaaggtgtttatgacaggttttcaaaaagatggtatacgaatagtttcgatatgttgttggatgctgtgtatatgtcaatggtaaggttgaagtatccagatgtggagattgtagctgctgaaacagggtggccttctcaaggtgaatcatatgagcctcaatgtacagtggaaaatgcag

SEQ ID NO: 3 - DNA, 2254 bp, Artificial Sequence
  Synthetic vector
cttcgtataatgtatgctatacgaaatcgcaagatattataaaaggagaggtttatatagatgtaataataataataacgtgtagtggaaggtaaaaaaggtagaggggcagaggaaaagcttatcgaaaatcgaagaaaaacccaaaggaggattcaaagaaagaagaaagatgacgacaaagaggcaaagttttgaaagtaaaagggtccgttaaaaagttgttttttcttttcttcttctttagtgaagtgagtagtacttgtagtatcaaacgttcaattgaaatcatagttaaaagttaatcatgagagcttagctaactgttgggacacttggactgaaattttcttacttacacttttatatttttctgttctttctctaacatttgttctcattgacaattcaccacacatatgagtggttcgctagttcgatatggccatgagttgagattatatatgctttggccaagtggatattatattgcaattaatctactatcagatgtggcaaccttggatttgctgaaaacggaaaatctgcattgggttggatttcttaaaagtaatgtatctaaaaaaatatagtcatgtttaacggtgctgaattttgccaactggacaagaatgcaaatgttacacattgtcatccaccaattaggaaatagatagtgatattcaaggataaggacttagggtctttcgagtcatttaaataaacttgttggaagatccatgaaactcatcaactcttctttctgtgtaatagctgcattcaagagtttttcagttactagtaaagtgtgaccccccatatcatgaatactagtagtactattcccaagtcgtctggactagtgaggaaaggtgcatggactgaagaagaagacgttcttttgagaaaatgtattgagaagttcggtgaaggaaagtggcatcaagttcctgtaagagctggtctgaatagatgcagaaagagctgcagacttaggtggttgaattatctaaggccacacataaagagaggggacttctctgaggatgaagtagatctcattttgaggcttcataagcttctaggcaacagatggtcacttatcgcgggtagacttccgggaagaacagcaaacgatgtcaaaaattactggaacacacacctgcagaggaagttaattgctcctccgcgacaagagataagaaagtgcagagcccttaagatcactgagaacaacatagtaagacctcgacctcggaccttctcaaataatgcacagaatatttcttggtgcagcaacaaaagtatcacaaccagcactatagataaagatgggagtaacaatgaatgtataaggattaatgataagaagccaatggccgaggagtcaagacacgatggagttcaatggtggactagtttactagctaactgcaacgaaaatgatgaaacagcagttgagaacatgtcatatgataagttaccgagtttgttgcacgaggaaatatcaccaacgataaatggtggaattagcaactgcatgcaagaaggacaaactggttgggatgacttttctgttgatattgatcacctatggaatctacttaactaggtttcataaccattaactatatattgagttatttttagaattgagttttcactaattatcgggttgtttgatggccttgtaaatttggctattgcaaattatggtaatcatatatgaaactttgtttggtcttcaataattttgaatggccataaaatttaaaatcctctacgtcgacaccagaagttcaaaacaagcttgttccagctatgagaactttttatcaagctcttgtacaatctggtttaaatggtattaaggtttcatcacctcattcgttgggtatactcttacgatccaatccaccaagtgctgcaagatttagacctggatgggatgttggtattcttgctccaatgcttcagtttctacgcgaaactaaaggaccgttcatggtaaacccatatccatactttggttacaacccgaaacaagaagactttcttcttttcaggaaaaacaaaggtgtttatgacaggttttcaaaaagatggtatacgaatagtttcgatatgttgttggatgctgtgtatatgtcaatggtaaggttgaagtatccagatgtggagattgtagctgctgaaacagggtggccttctcaaggtgaatcatatgagcctcaatgtacagtggaaaatgcag

SEQ ID NO: 4 - DNA, 5628 bp, Artificial Sequence
  Synthetic vector
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccccgggtgggctgatggaccggagtttgcgacccagtgtccgatccgacccggacatagctacacatacaagtttaaaataacaggacaagaaggaactctatggtggcatgcacacgtgtcatggcttagagccacagttcatggtgcactcattattcgcccaaaaaaaggccactcttatcctttccctaaaccttatagagaagttcctattctctagaaagtataggaacagaagggctaagaattccaaaatttcttcctgcacaggggataagtacaaatttgacataaaactcatagcaaagctcttcatacaaagaagaatgttaacaatttcaaatctcatcacacatatggggtcagccacaaaaataaagaacggttggaacggatctattatataatactaataaagaatagaaaaaggaaagtgagtgaggtgcgagggagagaatttgtttaatatcaaattcggctctgacttcaactgagtttaggcaattctgataaggcggggaaaatcatagtgctgagtctagaaaaatctcatgtagtgtgagataaacctcaacaaggacggtgagtccatagaggggtgtatgtgacaccccaacctcaacaaaagaaaacctcctttcaacaaggacattgggtccatagaggggatgtatgtgacatcataacttaagataaaaatgtaagaattattattaattatgtcttacttatggcccaagtttacttgtaacccaagtaataccataaataatatttaataaggaatagatctcgtccgtacattggttacttgatggacgtaccggattaagtcataacctgtataaattggtcctccctccacccattagggttaccacatattctctacaataattccatcgctgacggttgtggtaacataaagttagggaaaggaggtagaaaccaatttacgaccaacgcttccgcttctctctgtgatgatgtcttcctcatcaggtatgtgcccctaacgatctctatgtaagattatcgtgttcaagatcctgatattatgtattaaaacgtatttaatcttacatatggtagattgtttgagcggataatcttcatatacctcccctcaacaaggacatttgcggtgctaaacaatttcaagtctcatcacacatatatattatataatactaataaagaatagaaaaaggaaaggtaaacatcactaatgacagttgcggtgcaaagtgagtgaggtaataaacatcactaacttttattggttatgtcaaactcaaaataaaatttctcaacttgtttacgtgcctatatataccatgcttgttatatgctcaaagcaccaacaaaatttaaaaacactttgaacatttgcaaaatgagtactcctatgatgtgtacatctttgggagtaataaggaaaggttcatggactgaagaagaagatattcttttgaggaaatgtattgataagtatggagaaggaaagtggaatcttgttccaactagagctggattaaacagatgcaggaaaagttgtagactgaggtggctaaattatctaaggccacatatcaagagaggtgactttgattgggatgaagtggatctcatcttgagacttcataagctcttaggcaatcgatggtcacttattgctggtagacttccaggaaggacagctaacgatgtgaaaaactattggaacactaaccttctaaggaagctaaatactagtactaaatttgctcctcaaccacaagaaggaattaatactagtactattgctcctcaaccacaagaaggaattaagtgtgggaaagccaatgccataataagacctcaacctcagaaattcagaagctccatgaagattaatgtctcttggtgcaacaacaatagtatcgtaaataatgaagaagcatcgaaagataacaacgatatgcaatggtgggcaaatatactggaaaattgcaatgacattggagaaggagaagctgaaagaacactaccttcatgtaaggaaattaattgcaatgaaattgataaaacaccaagtttgttacatgatggaggcaactccacgcaacaaggacaaggtgatggtggttgggatgaattttctctagatgatatatggaatctacttaattagaagcttcttattaattcaaggtctcgggttgtagtagtaaccttactatgctaaataataaacgcttgcaatatttatgattgcacgcatttaagtatttcaacctcaaaataaaaggagtttgagggataaatttcaatagaaatgtctctctatgtaatgtgtgcttggattatgtaaccttttggttgtgttaaatatttaaataaattatcgttattttatgctatggctatttgaatcttcttttcaaagcaaaaagaaaaaaaatataatttgaagtgccaagttaatattatacaaataattatgatatgatataagagattcttcaactttagatatcctaaatgaatgagaaaaaaatagtactactttgaggcaagtgatttgatccatcagtatcaaggtaaattaacaatataatgtaattgaagttaacaggacaagaaggaactctatggtggcatgcacacgtgtcatggcttagagccacagttcatggtgcactcattattcgcccaaaaaaaggccactcttatcctttccctaaaccttatagagaagttcctattctctagaaagtataggaacagaagggctaagaattccaaaatttcttcctgcacaggggataagtacaaatttgacataaaactcatagcaaagctcttcatacaaagaagaatgttgtgcaagaggaccaatgagcacattaaaaatataggtgctttggaaggtcatgactcatgtgatataatatgtagtcattgaattaaacccgggccaatcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 5 - DNA, 2534 bp, Artificial Sequence
  Synthetic vector
gttaacaggacaagaaggaactctatggtggcatgcacacgtgtcatggcttagagccacagttcatggtgcactcattattcgcccaaaaaaaggccactcttatcctttccctaaaccttatagagaagttcctattctctagaaagtataggaacagaagggctaagaattccaaaatttcttcctgcacaggggataagtacaaatttgacataaaactcatagcaaagctcttcatacaaagaagaatgttaacaatttcaaatctcatcacacatatggggtcagccacaaaaataaagaacggttggaacggatctattatataatactaataaagaatagaaaaaggaaagtgagtgaggtgcgagggagagaatttgtttaatatcaaattcggctctgacttcaactgagtttaggcaattctgataaggcggggaaaatcatagtgctgagtctagaaaaatctcatgtagtgtgagataaacctcaacaaggacggtgagtccatagaggggtgtatgtgacaccccaacctcaacaaaagaaaacctcctttcaacaaggacattgggtccatagaggggatgtatgtgacatcataacttaagataaaaatgtaagaattattattaattatgtcttacttatggcccaagtttacttgtaacccaagtaataccataaataatatttaataaggaatagatctcgtccgtacattggttacttgatggacgtaccggattaagtcataacctgtataaattggtcctccctccacccattagggttaccacatattctctacaataattccatcgctgacggttgtggtaacataaagttagggaaaggaggtagaaaccaatttacgaccaacgcttccgcttctctctgtgatgatgtcttcctcatcaggtatgtgcccctaacgatctctatgtaagattatcgtgttcaagatcctgatattatgtattaaaacgtatttaatcttacatatggtagattgtttgagcggataatcttcatatacctcccctcaacaaggacatttgcggtgctaaacaatttcaagtctcatcacacatatatattatataatactaataaagaatagaaaaaggaaaggtaaacatcactaatgacagttgcggtgcaaagtgagtgaggtaataaacatcactaacttttattggttatgtcaaactcaaaataaaatttctcaacttgtttacgtgcctatatataccatgcttgttatatgctcaaagcaccaacaaaatttaaaaacactttgaacatttgcaaaatgagtactcctatgatgtgtacatctttgggagtaataaggaaaggttcatggactgaagaagaagatattcttttgaggaaatgtattgataagtatggagaaggaaagtggaatcttgttccaactagagctggattaaacagatgcaggaaaagttgtagactgaggtggctaaattatctaaggccacatatcaagagaggtgactttgattgggatgaagtggatctcatcttgagacttcataagctcttaggcaatcgatggtcacttattgctggtagacttccaggaaggacagctaacgatgtgaaaaactattggaacactaaccttctaaggaagctaaatactagtactaaatttgctcctcaaccacaagaaggaattaatactagtactattgctcctcaaccacaagaaggaattaagtgtgggaaagccaatgccataataagacctcaacctcagaaattcagaagctccatgaagattaatgtctcttggtgcaacaacaatagtatcgtaaataatgaagaagcatcgaaagataacaacgatatgcaatggtgggcaaatatactggaaaattgcaatgacattggagaaggagaagctgaaagaacactaccttcatgtaaggaaattaattgcaatgaaattgataaaacaccaagtttgttacatgatggaggcaactccacgcaacaaggacaaggtgatggtggttgggatgaattttctctagatgatatatggaatctacttaattagaagcttcttattaattcaaggtctcgggttgtagtagtaaccttactatgctaaataataaacgcttgcaatatttatgattgcacgcatttaagtatttcaacctcaaaataaaaggagtttgagggataaatttcaatagaaatgtctctctatgtaatgtgtgcttggattatgtaaccttttggttgtgttaaatatttaaataaattatcgttattttatgctatggctatttgaatcttcttttcaaagcaaaaagaaaaaaaatataatttgaagtgccaagttaatattatacaaataattatgatatgatataagagattcttcaactttagatatcctaaatgaatgagaaaaaaatagtactactttgaggcaagtgatttgatccatcagtatcaaggtaaattaacaatataatgtaattgaa

SEQ ID NO: 6 - DNA, 2274 bp, Artificial Sequence
  Synthetic vector
aagctttaacgagatagaaaattatattactccgttttgttcattacttaacaaatgcaacagtatcttgtaccaaatcctttctctcttttcaaacttttctatttggctgttgacagagtaatcaggatacaaaccacaagtatttaattgactcatccaccagatattatgatttatgaatcctcgaaaagcctatccattaagttctcatctatggatatacttgacagtttcttcctatttgggtatttttttttcctgccaagtggaacggagacatgttatgttgtatacgggaagctcgttaaaaaaaaaaatacaataggaagaaatgtaacaaacattgaatgttgtttttaaccatccttccttttagcagtgtatcaattttgtaatagaaccatgcatctcaatcttaatactaaaaaatgcaacaaaattctagtggagggaccagtaccagtacattagatattattttttattactataataatattttaattaacacgagacataggaatgtcaagtggtagcggtaggagggagttggtttagttttttagatactaggagacagaaccggaggggcccattgcaaggcccaagttgaagtccagccgtgaatcaacaaagagagggcccataatactgtcgatgagcatttccctataatacagtgtccacagttgccttccgctaagggatagccacccgctattctcttgacacgtgtcactgaaacctgctacaaataaggcaggcacctcctcattctcacactcactcactcacacagctcaacaagtggtaacttttactcatctcctccaattatttctgatttcatgcatgtttccctacattctattatgaatcgtgttatggtgtataaacgttgtttcatatctcatctcatctattctgattttgattctcttgcctactgaatttgaccctactgtaatcggtgataaatgtgaatgcttcctcttcttcttcttcttctcagaaatcaatttctgttttgtttttgttcatctgtagcttggtagattcccctttttgtagacatgagtactcctatgatgtgtacatctttgggagtaataaggaaaggttcatggactgaagaagaagatattcttttgaggaaatgtattgataagtatggagaaggaaagtggaatcttgttccaactagagctggattaaacagatgcaggaaaagttgtagactgaggtggctaaattatctaaggccacatatcaagagaggtgactttgattgggatgaagtggatctcatcttgagacttcataagctcttaggcaatcgatggtcacttattgctggtagacttccaggaaggacagctaacgatgtgaaaaactattggaacactaaccttctaaggaagctaaatactagtactaaatttgctcctcaaccacaagaaggaattaatactagtactattgctcctcaaccacaagaaggaattaagtgtgggaaagccaatgccataataagacctcaacctcagaaattcagaagctccatgaagattaatgtctcttggtgcaacaacaatagtatcgtaaataatgaagaagcatcgaaagataacaacgatatgcaatggtgggcaaatatactggaaaattgcaatgacattggagaaggagaagctgaaagaacactaccttcatgtaaggaaattaattgcaatgaaattgataaaacaccaagtttgttacatgatggaggcaactccacgcaacaaggacaaggtgatggtggttgggatgaattttctctagatgatatatggaatctacttaattagatccttgtttcaacaataagatcattaagcaaacgtatttactagcgaactatgtagaaccctattatggggtctcaatcatctacaaaatgattggtttttgctggggagcagcagcatattaggctgtaaaatcctggttaatgtttttgtaggtaagggctatttaaggtggtgtggatcaaagtcaatagaaaatagttattactagcgtttgcaactaaatacttagtaatgtagcataaataatactagtagctaatatatatgcgtgaatttgttgtaccttttcttgcataattatttgcagtacatatataatgaaaattacccaaggaatcaatgtttcttgctccgtcctcctttgatgattttttactcaatacagagctagtgtgttaagttataaattttgtttaaa

SEQ ID NO: 7 - DNA, 2199 bp, Artificial Sequence
  Synthetic vector
gtggtgagctaaacaatttcaaatctcatcacacatatggggtcagccacaaaaataaagaacggttggaacggatctattatataatactaataaagaatagaaaaaggaaagtgagtgaggtgcgagggagagaatttgtttaatatcaaattcggctctgacttcaactgagtttaggcaattctgataaggcggggaaaatcatagtgctgagtctagaaaaatctcatgtagtgtgagataaacctcaacaaggacggtgagtccatagaggggtgtatgtgacaccccaacctcaacaaaagaaaacctcctttcaacaaggacattgggtccatagaggggatgtatgtgacatcataacttaagataaaaatgtaagaattattattaattatgtcttacttatggcccaagtttacttgtaacccaagtaataccataaataatatttaataaggaatagatctcgtccgtacattggttacttgatggacgtaccggattaagtcataacctgtataaattggtcctccctccacccattagggttaccacatattctctacaataattccatcgctgacggttgtggtaacataaagttagggaaaggaggtagaaaccaatttacgaccaacgcttccgcttctctctgtgatgatgtcttcctcatcaggtatgtgcccctaacgatctctatgtaagattatcgtgttcaagatcctgatattatgtattaaaacgtatttaatcttacatatggtagattgtttgagcggataatcttcatatacctcccctcaacaaggacatttgcggtgctaaacaatttcaagtctcatcacacatatatattatataatactaataaagaatagaaaaaggaaaggtaaacatcactaatgacagttgcggtgcaaagtgagtgaggtaataaacatcactaacttttattggttatgtcaaactcaaaataaaatttctcaacttgtttacgtgcctatatataccatgcttgttatatgctcaaagcaccaacaaaatttaaaaacactttgaacatttgcaaaatggatgagtactcctatgatgtgtacatctttgggagtaataaggaaaggttcatggactgaagaagaagatattcttttgaggaaatgtattgataagtatggagaaggaaagtggaatcttgttccaactagagctggattaaacagatgcaggaaaagttgtagactgaggtggctaaattatctaaggccacatatcaagagaggtgactttgattgggatgaagtggatctcatcttgagacttcataagctcttaggcaatcgatggtcacttattgctggtagacttccaggaaggacagctaacgatgtgaaaaactattggaacactaaccttctaaggaagctaaatactagtactaaatttgctcctcaaccacaagaaggaattaatactagtactattgctcctcaaccacaagaaggaattaagtgtgggaaagccaatgccataataagacctcaacctcagaaattcagaagctccatgaagattaatgtctcttggtgcaacaacaatagtatcgtaaataatgaagaagcatcgaaagataacaacgatatgcaatggtgggcaaatatactggaaaattgcaatgacattggagaaggagaagctgaaagaacactaccttcatgtaaggaaattaattgcaatgaaattgataaaacaccaagtttgttacatgatggaggcaactccacgcaacaaggacaaggtgatggtggttgggatgaattttctctagatgatatatggaatctacttaattagagcttcttattaattcaaggtctcgggttgtagtagtaaccttactatgctaaataataaacgcttgcaatatttatgattgcacgcatttaagtatttcaacctcaaaataaaaggagtttgagggataaatttcaatagaaatgtctctctatgtaatgtgtgcttggattatgtaaccttttggttgtgttaaatatttaaataaattatcgttattttatgctatggctatttgaatcttcttttcaaagcaaaaagaaaaaaaatataatttgaagtgccaagttaatattatacaaataattatgatatgatataagagattcttcaactttagat

SEQ ID NO: 8 - DNA, 7578 bp, Artificial Sequence
  Synthetic vector
agggcgaattgggcccgacgtcgcatgctcccggccgccatggccgcgggtatcactatgcggccgcctgcaggtcgacaaatccagccatctctgctatccacgcctggagagcagcagctgatgaactggattcacaacgaggctcatttatgagttcccacgcaaatatggcgggctcttctgaatatttaactccacttatagagttttttcttgtcacaatagccttgacaaaagacttgtagtatgctttgaccgttggattggtaaagaatgcatctcttgatgaggacacattggtacctgcttctcgcgcccacctcatatattgagcagtgccaccatatgcttttaaattgtttacacttgtatatatcctgtcctctcagagctggatttgcatcaggatgaaaaacggaaagagcactcattgcactgacaagaacccccattggatgagcatcatggggcattgactgtatgatatcgattcaaggttacagcgagccgagtgatggttctaggccggtttcagatactgttaggagtagttccggtgtcggaagagttgatgctgatacggcgttgtacacggagctttggcgttcatgtgccggtccacttgtgacagtacctagagagggtgagctcgtgtactatttccctcaaggacatatcgagcaggttgaagcatcaacaaatcaagtggctgaccagcagatgccttcgtataatgtatgctatacgaattcgggttactacctgtacatacaatctccaagagcagaaatgcctcaaaaataaactactcctatgaatcaactttgtgcaccatataatattttcataagtccaaaatactggctctaaaaataactaatcagctaaaagagaagatagaacgaggtcttcgactgttttgccgttcttttgttaacccccagcctttccagagtttctctgtaatgccttccttgtcattactaggatgtggttgtaaaagtgagcatgttgttcttctcgcaataggggaccatctcctacttctcgaactagccttgagtggagttttcctcgatggacctgattctgatgcaacagttgcaggtgactctagacctccattcaactgatatctaaggatcagctttaacgagatagaaaattatattactccgttttgttcattacttaacaaatgcaacagtatcttgtaccaaatcctttctctcttttcaaacttttctatttggctgttgacagagtaatcaggatacaaaccacaagtatttaattgactcatccaccagatattatgatttatgaatcctcgaaaagcctatccattaagttctcatctatggatatacttgacagtttcttcctatttgggtatttttttttcctgccaagtggaacggagacatgttatgttgtatacgggaagctcgttaaaaaaaaaaatacaataggaagaaatgtaacaaacattgaatgttgtttttaaccatccttccttttagcagtgtatcaattttgtaatagaaccatgcatctcaatcttaatactaaaaaatgcaacaaaattctagtggagggaccagtaccagtacattagatattattttttattactataataatattttaattaacacgagacataggaatgtcaagtggtagcggtaggagggagttggtttagttttttagatactaggagacagaaccggaggggcccattgcaaggcccaagttgaagtccagccgtgaatcaacaaagagagggcccataatactgtcgatgagcatttccctataatacagtgtccacagttgccttccgctaagggatagccacccgctattctcttgacacgtgtcactgaaacctgctacaaataaggcaggcacctcctcattctcacactcactcactcacacagctcaacaagtggtaacttttactcatctcctccaattatttctgatttcatgcatgtttccctacattctattatgaatcgtgttatggtgtataaacgttgtttcatatctcatctcatctattctgattttgattctcttgcctactgaatttgaccctactgtaatcggtgataaatgtgaatgcttcctcttcttcttcttcttctcagaaatcaatttctgttttgtttttgttcatctgtagcttggtagattcccctttttgtagacatgagtactcctatgatgtgtacatctttgggagtaataaggaaaggttcatggactgaagaagaagatattcttttgaggaaatgtattgataagtatggagaaggaaagtggaatcttgttccaactagagctggattaaacagatgcaggaaaagttgtagactgaggtggctaaattatctaaggccacatatcaagagaggtgactttgattgggatgaagtggatctcatcttgagacttcataagctcttaggcaatcgatggtcacttattgctggtagacttccaggaaggacagctaacgatgtgaaaaactattggaacactaaccttctaaggaagctaaatactagtactaaatttgctcctcaaccacaagaaggaattaatactagtactattgctcctcaaccacaagaaggaattaagtgtgggaaagccaatgccataataagacctcaacctcagaaattcagaagctccatgaagattaatgtctcttggtgcaacaacaatagtatcgtaaataatgaagaagcatcgaaagataacaacgatatgcaatggtgggcaaatatactggaaaattgcaatgacattggagaaggagaagctgaaagaacactaccttcatgtaaggaaattaattgcaatgaaattgataaaacaccaagtttgttacatgatggaggcaactccacgcaacaaggacaaggtgatggtggttgggatgaattttctctagatgatatatggaatctacttaattagatccttgtttcaacaataagatcattaagcaaacgtatttactagcgaactatgtagaaccctattatggggtctcaatcatctacaaaatgattggtttttgctggggagcagcagcatattaggctgtaaaatcctggttaatgtttttgtaggtaagggctatttaaggtggtgtggatcaaagtcaatagaaaatagttattactagcgtttgcaactaaatacttagtaatgtagcataaataatactagtagctaatatatatgcgtgaatttgttgtaccttttcttgcataattatttgcagtacatatataatgaaaattacccaaggaatcaatgtttcttgctccgtcctcctttgatgattttttactcaatacagagctagtgtgttaagttataaattttgtttccggcatcaccatctatatagcatattgatgacctaacaggtgctgtgttgagatagcctggatcataaagcttaagacctttatcattctgtcctgttgttatcttctttaagtcggtggctttgatagtgccatcctcagaaacctgaaccggatacttcttaccggtccgctcatcgattcaaggttacagcgagccgagtgatggttctaggccggtttcagatactgttaggagtagttccggtgtcggaagagttgatgctgatacggcgttgtacacggagctttggcgttcatgtgccggtccacttgtgacagtacctagagagggtgagctcgtgtactatttccctcaaggacatatcgagcaggttgaagcatcaacaaatcaagtggctgaccagcagatgccttcgtataatgtatgctatacgaattcgggttactacctgtacatacaatctccaagagcagaaatgcctcaaaaataaactactcctatgaatcaactttgtgcaccatataatattttcataagtccaaaatactggctctaaaaataactaatcagctaaaagagaagatagaacgaggtcttcgactgttttgccgttcttttgttaacccccagcctttccagagtttctctgtaatgccttccttgtcattactaggatgtggttgtaaaagtgagcatgttgttcttctcgcaataggggaccatctcctacttctcgaactagccttgagtggagttttcctcgatggacctgattctgatgcaacagttgcaggtgactctagaagtttgaaggttgttaaatcggagacgttgagtgaagctgcaaggtgagctgaaagcaccgccaatctccctcgagcaactgctgaagaattcccgtttaccccaaaatatatcctgcctctgtcaacatctattaaattgtttccccattttagagttggttgcattttacgacctaaattaagaggtcttatagtatcagcaactgagactgatgtagcagtggaagaagtagaagcaactgctacagatgatggatcaggtgaagtctcagaagcttcatctgatgctgctgaaacaagtcaagaatcctccatatcagatgtgagtcctacatctgttcaatctaagcgttcaagacctgctagaaagagtgagatgcctcctgtgaagaatgaaaaccttattccaggtgcaacttttactgtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactat

SEQ ID NO: 9 - DNA, 7507 bp, Artificial Sequence
  Synthetic vector
gggcgaattgggcccgacgtcgcatgctcccggccgccatggccgcgggtatcactatgcggccgcctgcaggtcgacaaatccagccatctctgctatccacgcctggagagcagcagctgatgaactggattcacaacgaggctcatttatgagttcccacgcaaatatggcgggctcttctgaatatttaactccacttatagagttttttcttgtcacaatagccttgacaaaagacttgtagtatgctttgaccgttggattggtaaagaatgcatctcttgatgaggacacattggtacctgcttctcgcgcccacctcatatattgagcagtgccaccatatgcttttaaattgtttacacttgtatatatcctgtcctctcagagctggatttgcatcaggatgaaaaacggaaagagcactcattgcactgacaagaacccccattggatgagcatcatggggcattgactgtatgatatcgattcaaggttacagcgagccgagtgatggttctaggccggtttcagatactgttaggagtagttccggtgtcggaagagttgatgctgatacggcgttgtacacggagctttggcgttcatgtgccggtccacttgtgacagtacctagagagggtgagctcgtgtactatttccctcaaggacatatcgagcaggttgaagcatcaacaaatcaagtggctgaccagcagatgccttcgtataatgtatgctatacgaattcgggttactacctgtacatacaatctccaagagcagaaatgcctcaaaaataaactactcctatgaatcaactttgtgcaccatataatattttcataagtccaaaatactggctctaaaaataactaatcagctaaaagagaagatagaacgaggtcttcgactgttttgccgttcttttgttaacccccagcctttccagagtttctctgtaatgccttccttgtcattactaggatgtggttgtaaaagtgagcatgttgttcttctcgcaataggggaccatctcctacttctcgaactagccttgagtggagttttcctcgatggacctgattctgatgcaacagttgcaggtgactctagacctccattcaactgatatctaaggatcgtggtgagctaaacaatttcaaatctcatcacacatatggggtcagccacaaaaataaagaacggttggaacggatctattatataatactaataaagaatagaaaaaggaaagtgagtgaggtgcgagggagagaatttgtttaatatcaaattcggctctgacttcaactgagtttaggcaattctgataaggcggggaaaatcatagtgctgagtctagaaaaatctcatgtagtgtgagataaacctcaacaaggacggtgagtccatagaggggtgtatgtgacaccccaacctcaacaaaagaaaacctcctttcaacaaggacattgggtccatagaggggatgtatgtgacatcataacttaagataaaaatgtaagaattattattaattatgtcttacttatggcccaagtttacttgtaacccaagtaataccataaataatatttaataaggaatagatctcgtccgtacattggttacttgatggacgtaccggattaagtcataacctgtataaattggtcctccctccacccattagggttaccacatattctctacaataattccatcgctgacggttgtggtaacataaagttagggaaaggaggtagaaaccaatttacgaccaacgcttccgcttctctctgtgatgatgtcttcctcatcaggtatgtgcccctaacgatctctatgtaagattatcgtgttcaagatcctgatattatgtattaaaacgtatttaatcttacatatggtagattgtttgagcggataatcttcatatacctcccctcaacaaggacatttgcggtgctaaacaatttcaagtctcatcacacatatatattatataatactaataaagaatagaaaaaggaaaggtaaacatcactaatgacagttgcggtgcaaagtgagtgaggtaataaacatcactaacttttattggttatgtcaaactcaaaataaaatttctcaacttgtttacgtgcctatatataccatgcttgttatatgctcaaagcaccaacaaaatttaaaaacactttgaacatttgcaaaatggatgagtactcctatgatgtgtacatctttgggagtaataaggaaaggttcatggactgaagaagaagatattcttttgaggaaatgtattgataagtatggagaaggaaagtggaatcttgttccaactagagctggattaaacagatgcaggaaaagttgtagactgaggtggctaaattatctaaggccacatatcaagagaggtgactttgattgggatgaagtggatctcatcttgagacttcataagctcttaggcaatcgatggtcacttattgctggtagacttccaggaaggacagctaacgatgtgaaaaactattggaacactaaccttctaaggaagctaaatactagtactaaatttgctcctcaaccacaagaaggaattaatactagtactattgctcctcaaccacaagaaggaattaagtgtgggaaagccaatgccataataagacctcaacctcagaaattcagaagctccatgaagattaatgtctcttggtgcaacaacaatagtatcgtaaataatgaagaagcatcgaaagataacaacgatatgcaatggtgggcaaatatactggaaaattgcaatgacattggagaaggagaagctgaaagaacactaccttcatgtaaggaaattaattgcaatgaaattgataaaacaccaagtttgttacatgatggaggcaactccacgcaacaaggacaaggtgatggtggttgggatgaattttctctagatgatatatggaatctacttaattagagcttcttattaattcaaggtctcgggttgtagtagtaaccttactatgctaaataataaacgcttgcaatatttatgattgcacgcatttaagtatttcaacctcaaaataaaaggagtttgagggataaatttcaatagaaatgtctctctatgtaatgtgtgcttggattatgtaaccttttggttgtgttaaatatttaaataaattatcgttattttatgctatggctatttgaatcttcttttcaaagcaaaaagaaaaaaaatataatttgaagtgccaagttaatattatacaaataattatgatatgatataagagattcttcaactttagatccggcatcaccatctatatagcatattgatgacctaacaggtgctgtgttgagatagcctggatcataaagcttaagacctttatcattctgtcctgttgttatcttctttaagtcggtggctttgatagtgccatcctcagaaacctgaaccggatacttcttaccggtccgctcatcgattcaaggttacagcgagccgagtgatggttctaggccggtttcagatactgttaggagtagttccggtgtcggaagagttgatgctgatacggcgttgtacacggagctttggcgttcatgtgccggtccacttgtgacagtacctagagagggtgagctcgtgtactatttccctcaaggacatatcgagcaggttgaagcatcaacaaatcaagtggctgaccagcagatgccttcgtataatgtatgctatacgaattcgggttactacctgtacatacaatctccaagagcagaaatgcctcaaaaataaactactcctatgaatcaactttgtgcaccatataatattttcataagtccaaaatactggctctaaaaataactaatcagctaaaagagaagatagaacgaggtcttcgactgttttgccgttcttttgttaacccccagcctttccagagtttctctgtaatgccttccttgtcattactaggatgtggttgtaaaagtgagcatgttgttcttctcgcaataggggaccatctcctacttctcgaactagccttgagtggagttttcctcgatggacctgattctgatgcaacagttgcaggtgactctagaagtttgaaggttgttaaatcggagacgttgagtgaagctgcaaggtgagctgaaagcaccgccaatctccctcgagcaactgctgaagaattcccgtttaccccaaaatatatcctgcctctgtcaacatctattaaattgtttccccattttagagttggttgcattttacgacctaaattaagaggtcttatagtatcagcaactgagactgatgtagcagtggaagaagtagaagcaactgctacagatgatggatcaggtgaagtctcagaagcttcatctgatgctgctgaaacaagtcaagaatcctccatatcagatgtgagtcctacatctgttcaatctaagcgttcaagacctgctagaaagagtgagatgcctcctgtgaagaatgaaaaccttattccaggtgcaacttttactgtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 10 - DNA, 3106 bp, Artificial Sequence
  Synthetic vector
tcaaggttacagcgagccgagtgatggttctaggccggtttcagatactgttaggagtagttccggtgtcggaagagttgatgctgatacggcgttgtacacggagctttggcgttcatgtgccggtccacttgtgacagtacctagagagggtgagctcgtgtactatttccctcaaggacatatcgagcaggttgaagcatcaacaaatcaagtggctgaccagcagatgccttcgtataatgtatgctatacgaattcgggttactacctgtacatacaatctccaagagcagaaatgcctcaaaaataaactactcctatgaatcaactttgtgcaccatataatattttcataagtccaaaatactggctctaaaaataactaatcagctaaaagagaagatagaacgaggtcttcgactgttttgccgttcttttgttaacccccagcctttccagagtttctctgtaatgccttccttgtcattactaggatgtggttgtaaaagtgagcatgttgttcttctcgcaataggggaccatctcctacttctcgaactagccttgagtggagttttcctcgatggacctgattctgatgcaacagttgcaggtgactctagacctccattcaactgatatctaaggatcagctttaacgagatagaaaattatattactccgttttgttcattacttaacaaatgcaacagtatcttgtaccaaatcctttctctcttttcaaacttttctatttggctgttgacagagtaatcaggatacaaaccacaagtatttaattgactcatccaccagatattatgatttatgaatcctcgaaaagcctatccattaagttctcatctatggatatacttgacagtttcttcctatttgggtatttttttttcctgccaagtggaacggagacatgttatgttgtatacgggaagctcgttaaaaaaaaaaatacaataggaagaaatgtaacaaacattgaatgttgtttttaaccatccttccttttagcagtgtatcaattttgtaatagaaccatgcatctcaatcttaatactaaaaaatgcaacaaaattctagtggagggaccagtaccagtacattagatattattttttattactataataatattttaattaacacgagacataggaatgtcaagtggtagcggtaggagggagttggtttagttttttagatactaggagacagaaccggaggggcccattgcaaggcccaagttgaagtccagccgtgaatcaacaaagagagggcccataatactgtcgatgagcatttccctataatacagtgtccacagttgccttccgctaagggatagccacccgctattctcttgacacgtgtcactgaaacctgctacaaataaggcaggcacctcctcattctcacactcactcactcacacagctcaacaagtggtaacttttactcatctcctccaattatttctgatttcatgcatgtttccctacattctattatgaatcgtgttatggtgtataaacgttgtttcatatctcatctcatctattctgattttgattctcttgcctactgaatttgaccctactgtaatcggtgataaatgtgaatgcttcctcttcttcttcttcttctcagaaatcaatttctgttttgtttttgttcatctgtagcttggtagattcccctttttgtagatatgagtactcctatgatgtgtacatctttgggagtaataaggaaaggttcatggactgaagaagaagatattcttttgaggaaatgtattgataagtatggagaaggaaagtggaatcttgttccaactagagctggattaaacagatgcaggaaaagttgtagactgaggtggctaaattatctaaggccacatatcaagagaggtgactttgattgggatgaagtggatctcatcttgagacttcataagctcttaggcaatcgatggtcacttattgctggtagacttccaggaaggacagctaacgatgtgaaaaactattggaacactaaccttctaaggaagctaaatactagtactaaatttgctcctcaaccacaagaaggaattaatactagtactattgctcctcaaccacaagaaggaattaagtgtgggaaagccaatgccataataagacctcaacctcagaaattcagaagctccatgaagattaatgtctcttggtgcaacaacaatagtatcgtaaataatgaagaagcatcgaaagataacaacgatatgcaatggtgggcaaatatactggaaaattgcaatgacattggagaaggagaagctgaaagaacactaccttcatgtaaggaaattaattgcaatgaaattgataaaacaccaagtttgttacatgatggaggcaactccacgcaacaaggacaaggtgatggtggttgggatgaattttctctagatgatatatggaatctacttaattagatccttgtttcaacaataagatcattaagcaaacgtatttactagcgaactatgtagaaccctattatggggtctcaatcatctacaaaatgattggtttttgctggggagcagcagcatattaggctgtaaaatcctggttaatgtttttgtaggtaagggctatttaaggtggtgtggatcaaagtcaatagaaaatagttattactagcgtttgcaactaaatacttagtaatgtagcataaataatactagtagctaatatatatgcgtgaatttgttgtaccttttcttgcataattatttgcagtacatatataatgaaaattacccaaggaatcaatgtttcttgctccgtcctcctttgatgattttttactcaatacagagctagtgtgttaagttataaattttgtttccggcatcaccatctatatagcatattgatgacctaacaggtgctgtgttgagatagcctggatcataaagcttaagacctttatcattctgtcctgttgttatcttctttaagtcggtggctttgatagtgccatcctcagaaacctgaaccggatacttcttaccggtccgctcatcgat

SEQ ID NO: 11 - DNA, 3035 bp, Artificial Sequence
  Synthetic vector
tcaaggttacagcgagccgagtgatggttctaggccggtttcagatactgttaggagtagttccggtgtcggaagagttgatgctgatacggcgttgtacacggagctttggcgttcatgtgccggtccacttgtgacagtacctagagagggtgagctcgtgtactatttccctcaaggacatatcgagcaggttgaagcatcaacaaatcaagtggctgaccagcagatgccttcgtataatgtatgctatacgaattcgggttactacctgtacatacaatctccaagagcagaaatgcctcaaaaataaactactcctatgaatcaactttgtgcaccatataatattttcataagtccaaaatactggctctaaaaataactaatcagctaaaagagaagatagaacgaggtcttcgactgttttgccgttcttttgttaacccccagcctttccagagtttctctgtaatgccttccttgtcattactaggatgtggttgtaaaagtgagcatgttgttcttctcgcaataggggaccatctcctacttctcgaactagccttgagtggagttttcctcgatggacctgattctgatgcaacagttgcaggtgactctagacctccattcaactgatatctaaggatcgtggtgagctaaacaatttcaaatctcatcacacatatggggtcagccacaaaaataaagaacggttggaacggatctattatataatactaataaagaatagaaaaaggaaagtgagtgaggtgcgagggagagaatttgtttaatatcaaattcggctctgacttcaactgagtttaggcaattctgataaggcggggaaaatcatagtgctgagtctagaaaaatctcatgtagtgtgagataaacctcaacaaggacggtgagtccatagaggggtgtatgtgacaccccaacctcaacaaaagaaaacctcctttcaacaaggacattgggtccatagaggggatgtatgtgacatcataacttaagataaaaatgtaagaattattattaattatgtcttacttatggcccaagtttacttgtaacccaagtaataccataaataatatttaataaggaatagatctcgtccgtacattggttacttgatggacgtaccggattaagtcataacctgtataaattggtcctccctccacccattagggttaccacatattctctacaataattccatcgctgacggttgtggtaacataaagttagggaaaggaggtagaaaccaatttacgaccaacgcttccgcttctctctgtgatgatgtcttcctcatcaggtatgtgcccctaacgatctctatgtaagattatcgtgttcaagatcctgatattatgtattaaaacgtatttaatcttacatatggtagattgtttgagcggataatcttcatatacctcccctcaacaaggacatttgcggtgctaaacaatttcaagtctcatcacacatatatattatataatactaataaagaatagaaaaaggaaaggtaaacatcactaatgacagttgcggtgcaaagtgagtgaggtaataaacatcactaacttttattggttatgtcaaactcaaaataaaatttctcaacttgtttacgtgcctatatataccatgcttgttatatgctcaaagcaccaacaaaatttaaaaacactttgaacatttgcaaaaagcatgagtactcctatgatgtgtacatctttgggagtaataaggaaaggttcatggactgaagaagaagatattcttttgaggaaatgtattgataagtatggagaaggaaagtggaatcttgttccaactagagctggattaaacagatgcaggaaaagttgtagactgaggtggctaaattatctaaggccacatatcaagagaggtgactttgattgggatgaagtggatctcatcttgagacttcataagctcttaggcaatcgatggtcacttattgctggtagacttccaggaaggacagctaacgatgtgaaaaactattggaacactaaccttctaaggaagctaaatactagtactaaatttgctcctcaaccacaagaaggaattaatactagtactattgctcctcaaccacaagaaggaattaagtgtgggaaagccaatgccataataagacctcaacctcagaaattcagaagctccatgaagattaatgtctcttggtgcaacaacaatagtatcgtaaataatgaagaagcatcgaaagataacaacgatatgcaatggtgggcaaatatactggaaaattgcaatgacattggagaaggagaagctgaaagaacactaccttcatgtaaggaaattaattgcaatgaaattgataaaacaccaagtttgttacatgatggaggcaactccacgcaacaaggacaaggtgatggtggttgggatgaattttctctagatgatatatggaatctacttaattagagcttcttattaattcaaggtctcgggttgtagtagtaaccttactatgctaaataataaacgcttgcaatatttatgattgcacgcatttaagtatttcaacctcaaaataaaaggagtttgagggataaatttcaatagaaatgtctctctatgtaatgtgtgcttggattatgtaaccttttggttgtgttaaatatttaaataaattatcgttattttatgctatggctatttgaatcttcttttcaaagcaaaaagaaaaaaaatataatttgaagtgccaagttaatattatacaaataattatgatatgatataagagattcttcaactttagatccggcatcaccatctatatagcatattgatgacctaacaggtgctgtgttgagatagcctggatcataaagcttaagacctttatcattctgtcctgttgttatcttctttaagtcggtggctttgatagtgccatcctcagaaacctgaaccggatacttcttaccggtccgctcatcgat

SEQ ID NO: 12 - DNA, 248 bp, Artificial Sequence
  Synthetic vector
gtctagagcggccgcataacttcgtatagcatacattatacgaacggtaagatctctgcagaagcttgacgtcggtaccactagttgtacacccggggaattctccggacaattgcgtacggagctcctcgagcctaggtgacaggatatattggcgggtaaactaagtcgctgtatgtgtttgtttgatcgatgggccctaccgttcgtatagcatacattatacgaagttatgcggccgcgtcgac

SEQ ID NO: 13 - DNA, 29 bp, Artificial Sequence
  Synthetic primer
ccacatgtccaatttactgaccgttacac

SEQ ID NO: 14 - DNA, 18 bp, Artificial Sequence
  Synthetic primer
gtcgacgcggccgctcta

SEQ ID NO: 15 - DNA, 12674 bp, Artificial Sequence
  Synthetic vector
gtcgacaggaaacagctatgaccatgattacgccaagctcgaaattaaccctcactaaagggaacaaaagctggagctccaccgcggtggcggccgcataacttcgtataatgtatgctatacgaacggtagggcccatcgatcaaacaaacacatacagcgacttagtttacccgccaatatatcctgtcacctaggctcgaggagctccgtacgcaattgtccggagaattccccgggtgtacaactagtggtaccgacgtcaagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccagatctcgactctagtcgagggcccatgggagcttggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggacccaagctagcttcgacggatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagcttctgcagagatcttaccgttcgtataatgtatgctatacgaagttatgcggccgctctagaaactcaatggtgatggtgatgatgaccggtacgcgtagaatcgagaccgaggagagggttagggataggcttaccttcagtcgacgcggccgctctagactaatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatccttagcgccgtaaatcaatcggtgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagttacccccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacgaacctggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcctgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatgtggcatgggtatgtatatctccttcttaaagttaaacaaaattatttctagcccaaaaaaacgggtatggagaaacagtagagagttgcgataaaaagcgtcaggtaggatccgctaatcttatggataaaaatgctatggcatagcaaagtgtgacgccgtgcaaataatcaatgtggacttttctgccgtgattatagacacttttgttacgcgtttttgtcatggctttggtcccgctttgttacagaatgcttttaataagcggggttaccggtttggttagcgagaagagccagtaaaagacgcagtgacggcaatgtctgatgcaatatggacaattggtttcttctctgaatggcgggagtatgaaaagtatggctgaagcgcaaaatgatcccctgctgccgggatactcgtttaatgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtttgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacggggttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgtatcagccaggcgaagctgcttttgagcaccacccggatgcctatcgccaccgtcggtcgcaatgttggttttgacgatcaactctatttctcgcgggtatttaaaaaatgcaccggggccagcccgagcgagttccgtgccggttgtgaagaaaaagtgaatgatgtagccgtcaagttgtcataattggtaacgaatcagacaattgacggcttgacggagtagcatagggtttgcagaatccctgcttcgtccatttgacaggcacattatgctagaactagtggatcccccgggctgcaggaattcgatatcaagcttatcgataccgtcgacctcgagggggggcccggtacccaattcgccctatagtgagtcgtattacaattcactggccgtcgttttacatcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccgggatggcgctaagaagctattgccgccgatcttcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatatcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaaacaagtggcagcaacggattcgcaaacctgtcacgccttttgtgccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaatggccaattttcgcttgccctgacagataacggcctgaagatcatgtatctaagcaactagcctgctctctaataaaatgttaggagcttggctgccatttttggggtgaggccgttcgcggccgaggggcgcagcccctggggggatgggaggcccgcgttagcgggccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagcgtcggaaagg

SEQ ID NO: 16 - DNA, 1916 bp, Artificial Sequence
  Synthetic vector
caaacaaacacatacagcgacttagtttacccgccaatatatcctgtcacctaggctcgaggagctccgtacgcaattgtccggagaattccccgggtgtacaactagtggtaccgacgtcaagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccagatctcgactctagtcgagggcccatgggagcttggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggacccaagctagcttcgacggatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagcttctgcagagatcttaccgttcgtataatgtatgctatacgaacggtagggcccatcgat

SEQ ID NO: 17 - DNA, 24 bp, Artificial Sequence
  Synthetic primer
ggttgggaagccctgcaaagtaaa

SEQ ID NO: 18 - DNA, 22 bp, Artificial Sequence
  Synthetic primer
tcgctgtatgtgtttgtttgat

SEQ ID NO: 19 - DNA, 24 bp, Artificial Sequence
  Synthetic primer
tcttgcgaacctcatcactcgttg

SEQ ID NO: 20 - DNA, 24 bp, Artificial Sequence
  Synthetic primer
ctaatccctaactgctggcggaaa

SEQ ID NO: 21 - DNA, 12661 bp, Artificial Sequence
  Synthetic vector
gtcgacaggaaacagctatgaccatgattacgccaagctcgaaattaaccctcactaaagggaacaaaagctggagctccaccgcggtggcggccgcataacttcgtatagcatacattatacgaacggtaagatctggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagcttgcatgcctgcagaagcttgacgtcggtaccactagttgtacacccggggaattctccggacaattgcgtacggagctcctcgagcctaggtgacaggatatattggcgggtaaactaagtcgctgtatgtgtttgtttgatcgataagcttggcaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatccgtcgaagctagcttgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccatgggccctaccgttcgtatagcatacattatacgaagttatgcggccgctctagaaactcaatggtgatggtgatgatgaccggtacgcgtagaatcgagaccgaggagagggttagggataggcttaccttcagtcgacgcggccgctctagactaatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatccttagcgccgtaaatcaatcggtgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagttacccccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacgaacctggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcctgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatgtggcatgggtatgtatatctccttcttaaagttaaacaaaattatttctagcccaaaaaaacgggtatggagaaacagtagagagttgcgataaaaagcgtcaggtaggatccgctaatcttatggataaaaatgctatggcatagcaaagtgtgacgccgtgcaaataatcaatgtggacttttctgccgtgattatagacacttttgttacgcgtttttgtcatggctttggtcccgctttgttacagaatgcttttaataagcggggttaccggtttggttagcgagaagagccagtaaaagacgcagtgacggcaatgtctgatgcaatatggacaattggtttcttctctgaatggcgggagtatgaaaagtatggctgaagcgcaaaatgatcccctgctgccgggatactcgtttaatgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtttgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacggggttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgtatcagccaggcgaagctgcttttgagcaccacccggatgcctatcgccaccgtcggtcgcaatgttggttttgacgatcaactctatttctcgcgggtatttaaaaaatgcaccggggccagcccgagcgagttccgtgccggttgtgaagaaaaagtgaatgatgtagccgtcaagttgtcataattggtaacgaatcagacaattgacggcttgacggagtagcatagggtttgcagaatccctgcttcgtccatttgacaggcacattatgctagaactagtggatcccccgggctgcaggaattcgatatcaagcttatcgataccgtcgacctcgagggggggcccggtacccaattcgccctatagtgagtcgtattacaattcactggccgtcgttttacatcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccgggatggcgctaagaagctattgccgccgatcttcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatatcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaaacaagtggcagcaacggattcgcaaacctgtcacgccttttgtgccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaatggccaattttcgcttgccctgacagataacggcctgaagatcatgtatctaagcaactagcctgctctctaataaaatgttaggagcttggctgccatttttggggtgaggccgttcgcggccgaggggcgcagcccctggggggatgggaggcccgcgttagcgggccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagcgtcggaaagg

SEQ ID NO: 22 - DNA, 1903 bp, Artificial Sequence
  Synthetic vector
caaacaaacacatacagcgacttagtttacccgccaatatatcctgtcacctaggctcgaggagctccgtacgcaattgtccggagaattccccgggtgtacaactagtggtaccgacgtcaagcttctgcaggcatgcaagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccagatcttaccgttcgtataatgtatgctatacgaacggtagggcccatgggagcttggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggacccaagctagcttcgacggatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagcttatcgat

SEQ ID NO: 23 - DNA, 20 bp, Artificial Sequence
  Synthetic primer
aggaagcggaacacgtagaa

SEQ ID NO: 24 - DNA, 23 bp, Artificial Sequence
  Synthetic primer
gcgggactctaatcataaaaacc

SEQ ID NO: 25 - DNA, 24 bp, Artificial Sequence
  Synthetic primer
ggttgggaagccctgcaaagtaaa

SEQ ID NO: 26 - DNA, 24 bp, Artificial Sequence
  Synthetic primer
tcttgcgaacctcatcactcgttg

SEQ ID NO: 27 - DNA, 24 bp, Artificial Sequence
  Synthetic primer
ctaatccctaactgctggcggaaa

SEQ ID NO: 28 - DNA, 2713 bp, Artificial Sequence
  Synthetic vector
ggatccgcggccgcgtcgacaggccactcttatcctttccctaaaccttatagagaagttcctattctctagaaagtataggaacagaagggctaagaattccaaaatttcttcctgcactttgcaaatgttcaaagtgtttttaaattttgttggtgctttgagcatataacaagcatggtatatataggcacgtaaacaagttgagaaattttattttgagtttgacataaccaataaaagttagtgatgtttattacctcactcactttgcaccgcaactgtcattagtgatgtttacctttcctttttctattctttattagtattatataatatatatgtgtgatgagacttgaaattgtttagcaccgcaaatgtccttgttgaggggaggtatatgaagattatccgctcaaacaatctaccatatgtaagattaaatacgttttaatacataatatcaggatcttgaacacgataatcttacatagagatcgttaggggcacatacctgatgaggaagacatcatcacagagagaagcggaagcgttggtcgtaaattggtttctacctcctttccctaactttatgttaccacaaccgtcagcgatggaattattgtagagaatatgtggtaaccctaatgggtggagggaggaccaatttatacaggttatgacttaatccggtacgtccatcaagtaaccaatgtacggacgagatctattccttattaaatattatttatggtattacttgggttacaagtaaacttgggccataagtaagacataattaataataattcttacatttttatcttaagttatgatgtcacatacatcccctctatggacccaatgtccttgttgaaaggaggttttcttttgttgaggttggggtgtcacatacacccctctatggactcaccgtccttgttgaggtttatctcacactacatgagatttttctagactcagcactatgattttccccgccttatcagaattgcctaaactcagttgaagtcagagccgaatttgatattaaacaaattctctccctcgcacctcactcactttcctttttctattctttattagtattatataatagatccgttccaaccgttctttatttttgtggctgaccccatatgtgtgatgagatttgaaattgttgtggtggtaagaagaagagcaacataatgaccgggattcatcttcatgatttcgttagcagtgacaggatatattgcatggtaaacatacgaaacattgtattccacattgatctgttcattaagagcagcttcagattgatcagtgaacttctgtctagcaagagaagcatgaggaacagtaggtacaagcataagctcaacttcaattacattatattgttaatttaccttgatactgatggatcaaatcacttgcctcaaagtagtactatttttttctcattcatttaggatatctaaagttgaagaatctcttatatcatatcataattatttgtataatattaacttggcacttcaaattatattttttttctttttgctttgaaaagaagattcaaatagccatagcataaaataacgataatttatttaaatatttaacacaaccaaaaggttacataatccaagcacacattacatagagagacatttctattgaaatttatccctcaaactccttttattttgaggttgaaatacttaaatgcgtgcaatcataaatattgcaagcgtttattatttagcatagtaaggttactactacaacccgagaccttgaattaataagaagcttctaattaagtagattccatatatcatctagagaaaattcatcccaaccaccatcaccttgtccttgttgcgtggagttgcctccatcatgtaacaaacttggtgttttatcaatttcattgcaattaatttccttacatgaaggtagtgttctttcagcttctccttctccaatgtcattgcaattttccagtatatttgcccaccattgcatatcgttgttatctttcgatgcttcttcattatttacgatactattgttgttgcaccaagagacattaatcttcatggagcttctgaatttctgaggttgaggtcttattatggcattggctttcccacacttaattccttcttgtggttgaggagcaatagtactagtattaattccttcttgtggttgaggagcaaatttagtactagtatttagcttccttagaaggttagtgttccaatagtttttcacatcgttagctgtccttcctggaagtctaccagcaataagtgaccatcgattgcctaagagcttatgaagtctcaagatgagatccacttcatcccaatcaaagtcacctctcttgatatgtggccttagataatttagccacctcagtctacaacttttcctgcatctgtttaatccagctctagttggaacaagattccactttccttctccatacttatcaatacatttcctcaaaagaatatcttcttcttcagtccatgaacctttccttattactcccaaagatgtacacatcataggagtactcataggccactcttatcctttccctaaaccttatagagaagttcctattctctagaaagtataggaacagaagggctaagaattccaaaatttcttcctgcacgtcgacggatcc

SEQ ID NO: 29 - DNA, 2581 bp, Artificial Sequence
  Synthetic vector
gagcttatgcttgtacctactgttcctcatgcttctcttgctagacagaagttcactgatcaatctgaagctgctcttaatgaacagatcaatgtggaatacaatgtttcgtatgtttaccatgcaatatatcctgtcactgctaacgaaatcatgaagatgaatcccggtcattatgttgctcttcttcttaccaccacaacaatttcaaatctcatcacacatatggggtcagccacaaaaataaagaacggttggaacggatctattatataatactaataaagaatagaaaaaggaaagtgagtgaggtgcgagggagagaatttgtttaatatcaaattcggctctgacttcaactgagtttaggcaattctgataaggcggggaaaatcatagtgctgagtctagaaaaatctcatgtagtgtgagataaacctcaacaaggacggtgagtccatagaggggtgtatgtgacaccccaacctcaacaaaagaaaacctcctttcaacaaggacattgggtccatagaggggatgtatgtgacatcataacttaagataaaaatgtaagaattattattaattatgtcttacttatggcccaagtttacttgtaacccaagtaataccataaataatatttaataaggaatagatctcgtccgtacattggttacttgatggacgtaccggattaagtcataacctgtataaattggtcctccctccacccattagggttaccacatattctctacaataattccatcgctgacggttgtggtaacataaagttagggaaaggaggtagaaaccaatttacgaccaacgcttccgcttctctctgtgatgatgtcttcctcatcaggtatgtgcccctaacgatctctatgtaagattatcgtgttcaagatcctgatattatgtattaaaacgtatttaatcttacatatggtagattgtttgagcggataatcttcatatacctcccctcaacaaggacatttgcggtgctaaacaatttcaagtctcatcacacatatatattatataatactaataaagaatagaaaaaggaaaggtaaacatcactaatgacagttgcggtgcaaagtgagtgaggtaataaacatcactaacttttattggttatgtcaaactcaaaataaaatttctcaacttgtttacgtgcctatatataccatgcttgttatatgctcaaagcaccaacaaaatttaaaaacactttgaacatttgcaaagtgcaggaagaaattttggaattcttagcccttctgttcctatactttctagagaataggaacttctctataaggtttagggaaaggataagagtggcctatgagtactcctatgatgtgtacatctttgggagtaataaggaaaggttcatggactgaagaagaagatattcttttgaggaaatgtattgataagtatggagaaggaaagtggaatcttgttccaactagagctggattaaacagatgcaggaaaagttgtagactgaggtggctaaattatctaaggccacatatcaagagaggtgactttgattgggatgaagtggatctcatcttgagacttcataagctcttaggcaatcgatggtcacttattgctggtagacttccaggaaggacagctaacgatgtgaaaaactattggaacactaaccttctaaggaagctaaatactagtactaaatttgctcctcaaccacaagaaggaattaatactagtactattgctcctcaaccacaagaaggaattaagtgtgggaaagccaatgccataataagacctcaacctcagaaattcagaagctccatgaagattaatgtctcttggtgcaacaacaatagtatcgtaaataatgaagaagcatcgaaagataacaacgatatgcaatggtgggcaaatatactggaaaattgcaatgacattggagaaggagaagctgaaagaacactaccttcatgtaaggaaattaattgcaatgaaattgataaaacaccaagtttgttacatgatggaggcaactccacgcaacaaggacaaggtgatggtggttgggatgaattttctctagatgatatatggaatctacttaattagaagcttcttattaattcaaggtctcgggttgtagtagtaaccttactatgctaaataataaacgcttgcaatatttatgattgcacgcatttaagtatttcaacctcaaaataaaaggagtttgagggataaatttcaatagaaatgtctctctatgtaatgtgtgcttggattatgtaaccttttggttgtgttaaatatttaaataaattatcgttattttatgctatggctatttgaatcttcttttcaaagcaaaaagaaaaaaaatataatttgaagtgccaagttaatattatacaaataattatgatatgatataagagattcttcaactttagatatcctaaatgaatgagaaaaaaatagtactactttgaggcaagtgatttgatccatcagtatcaaggtaaattaacaatataatgtaattgaagtt

SEQ ID NO: 30 - DNA, 4903 bp, Artificial Sequence
  Synthetic vector
ggatccagtattttggacttatgaaaatattatatggtgcacaaagttgattcataggagtagtttatttttgaggcatttctgctcttggagattgtatgtacaggtagtaacccgaattcgtatagcatacattatacgaaggcatctgctggtcagccacttgatttgttgatgcttcaacctgctcgatatgtccttgagggaaatagtacacgagctcaccctctctaggtactgtcacaagtggaccggcacatgaacgccaaagctccgtgtacaacgccgtatcagcatcaactcttccgacacgtggaagcggtggcggagcaaggattagtgggcggacacaaggtggtggtggtaagaagaagagcaacataatgaccgggattcatcttcatgatttcgttagcagtgacaggatatattgcatggtaaacatacgaaacattgtattccacattgatctgttcattaagagcagcttcagattgatcagtgaacttctgtctagcaagagaagcatgaggaacagtaggtacaagcataagctctttcttcacctcttcaaagggttcgaaaacaacacccgtcaacggtttgctgttcgaaccctttgaagcacatacgtagtgatcattactcctttgtttgttttatttgtcatgttagttcattaaaaagaaaatctctcttcttatcaattctgacgtgtttaatatcataagattaaagaatatttaaatatatctttaatttaaaaccacaaagttcaaatttcttcgttaacttaatttgtcaaatcaggctcaaagatcgtttttcatatcggaatgaggattttatttattcttttaaaaataaagaggtggtgagctaaacaatttcaaatctcatcacacatatggggtcagccacaaaaataaagaacggttggaacggatctattatataatactaataaagaatagaaaaaggaaagtgagtgaggtgcgagggagagaatttgtttaatatcaaattcggctctgacttcaactgagtttaggcaattctgataaggcggggaaaatcatagtgctgagtctagaaaaatctcatgtagtgtgagataaacctcaacaaggacggtgagtccatagaggggtgtatgtgacaccccaacctcaacaaaagaaaacctcctttcaacaaggacattgggtccatagaggggatgtatgtgacatcataacttaagataaaaatgtaagaattattattaattatgtcttacttatggcccaagtttacttgtaacccaagtaataccataaataatatttaataaggaatagatctcgtccgtacattggttacttgatggacgtaccggattaagtcataacctgtataaattggtcctccctccacccattagggttaccacatattctctacaataattccatcgctgacggttgtggtaacataaagttagggaaaggaggtagaaaccaatttacgaccaacgcttccgcttctctctgtgatgatgtcttcctcatcaggtatgtgcccctaacgatctctatgtaagattatcgtgttcaagatcctgatattatgtattaaaacgtatttaatcttacatatggtagattgtttgagcggataatcttcatatacctcccctcaacaaggacatttgcggtgctaaacaatttcaagtctcatcacacatatatattatataatactaataaagaatagaaaaaggaaaggtaaacatcactaatgacagttgcggtgcaaagtgagtgaggtaataaacatcactaacttttattggttatgtcaaactcaaaataaaatttctcaacttgtttacgtgcctatatataccatgcttgttatatgctcaaagcgctcatcacttcttatagccattttgcctcctttcacttctcacctttatcgacaacaccaacaatggcggctgctgcctcaccatctccatgtttctccaaaaccctacctccatcttcctccaaatcttccaccattcttcctagatctaccttccctttccacaatcaccctcaaaaagcctcaccccttcatctcacccacacccatcatcatcgtcgtggtttcgccgtttccaatgtcgtcatatccactaccacccataacgacgtttctgaacctgaaacattcgtttcccgtttcgcccctgacgaacccagaaagggttgtgatgttcttgtggaggcacttgaaagggagggggttacggatgtatttgcgtacccaggaggtgcttctatggagattcatcaggctttgacacgttcgaatattattcgtaatgtgctgccacgtcatgagcaaggtggtgtgtttgctgcagagggttacgcacgggcgactgggttccctggtgtttgcattgctacctctggtccgggagctacgaatcttgttagtggtcttgcggatgctttgttggatagtattccgattgttgctattacgggtcaagtgtcgaggaggatgattggtactgatgcgtttcaggaaacgcctattgttgaggtaacgagatctattacgaagcataattatcttgttatggatgtagaggatattcctagggttgttcgtgaagcgttttttctagcgaaatcgggacggcctgggccggttttgattgatgtacctaaggatattcagcaacaattggtgatacctaattgggatcagccaatgaggttgcctggttacatgtctaggttacctaaattgcctaatgagatgcttttggaacaaattattaggctgatttcggagtcgaagaagcctgttttgtatgtgggtggtgggtgtttgcaatcaagtgaggagctgagacgatttgtggagcttacgggtattcctgtggcgagtactttgatgggtcttggagcttttccaactggggatgagctttcccttcaaatgttgggtatgcatgggactgtgtatgctaattatgctgtggatggtagtgatttgttgcttgcatttggggtgaggtttgatgatcgagttactggtaaattggaagcttttgctagccgagcgaaaattgtccacattgatattgattcggctgagattggaaagaacaagcaacctcatgtttccatttgtgcagatatcaagttggcattacagggtttgaattccatattggagggtaaagaaggtaagctgaagttggacttttctgcttggagacaggagttaacggaacagaaggtgaagtacccattgagttttaagacttttggtgaagccatccctccacaatatgctattcaggttcttgatgagttaactaacggaaatgccattattagtactggtgtggggcaacaccagatgtgggctgcccaatactataagtacaaaaagccacaccaatggttgacatctggtggattaggagcaatgggatttggtttgcctgctgcaataggtgcggctgttggaagaccgggtgagattgtggttgacattgatggtgacgggagttttatcatgaatgtgcaggagttagcaacaattaaggtggagaatctcccagttaagattatgttgctgaataatcaacacttgggaatggtggttcaaagggaggatcgattctataaggctaacagagcacacacttacttgggtgatcctgctaatgaggaagagatcttccctaatatgttgaaattcgcagaggcttgtggcgtacctgctgcaagagtgtcacacagggatgatcttagagctgccattcaaaagatgttagacactcctgggccatacttgttggatgtgattgtacctcatcaggagcacgttctacctatgattcccagtggcggtgctttcaaagatgtgatcacagagggtgatgggagacgttcatattgacttttagaaactacataactagctctaggcattgtattatctaaaataaacttctattaagccaaaagtgttctatctgtctagtttaaactccgagcaaacaaagcttcttattaattcaaggtctcgggttgtagtagtaaccttactatgctaaataataaacgcttgcaatatttatgattgcacgcatttaagtatttcaacctcaaaataaaaggagtttgagggataaatttcaatagaaatgtctctctatgtaatgtgtgcttggattatgtaaccttttggttgtgttaaatatttaaataaattatcgttattttatgctatggctatttgaatcttcttttcaaagcaaaaagaaaaaaaatataatttgaagtgccaagttaatattatacaaataattatgatatgatataagagattcttcaactttagatatcctaaatgaatgagaaaaaaatagtactactttgaggcaagtgatttgatccatcagtatcaaggtaaattaacaatataatgtatacagagaaactctggaaaggctgggggttaacaaaagaacggcaaaacagtcgaagacctcgttctatcttctcttttagctgattagttatttttagagccagtattttggacttatgaaaatattatatggtgcacaaagttgattcataggagtagtttatttttgaggcatttctgctcttggagattgtatgtacaggtagtaacccgaattcgtatagcatacattatacgaaggcatctgctggtcagccacttgatttgttgatgcttcaacctgctcgatatgtccttgagggaaatagtacacgagctcaccctctctaggtactgtcacaagtggaccggcacatgaacgccaaagctccgtgtacaacgccgtatcagcatcaactcttccgacaccggatcc

SEQ ID NO: 31 - DNA, 4584 bp, Artificial Sequence
  Synthetic vector
tacagagaaactctggaaaggctgggggttaacaaaagaacggcaaaacagtcgaagacctcgttctatcttctcttttagctgattagttatttttagagccagtattttggacttatgaaaatattatatggtgcacaaagttgattcataggagtagtttatttttgaggcatttctgctcttggagattgtatgtacaggtagtaacccgaattcgtatagcatacattatacgaaggcatctgctggtcagccacttgatttgttgatgcttcaacctgctcgatatgtccttgagggaaatagtacacgagctcaccctctctaggtactgtcacaagtggaccggcacatgaacgccaaagctccgtgtacaacgccgtatcagcatcaactcttccgacacgtggaagcggtggcggagcaaggattagtgggcggacacaaggtggtggtggtaagaagaagagcaacataatgaccgggattcatcttcatgatttcgttagcagtgacaggatatattgcatggtaaacatacgaaacattgtattccacattgatctgttcattaagagcagcttcagattgatcagtgaacttctgtctagcaagagaagcatgaggaacagtaggtacaagcataagctctttcttcacctcttcaaagggttcgaaaacaacacccgtcaacggtttgctgttcgaaccctttgaagcacatacgtagtgatcattactcctttgtttgttttatttgtcatgttagttcattaaaaagaaaatctctcttcttatcaattctgacgtgtttaatatcataagattaaagaatatttaaatatatctttaatttaaaaccacaaagttcaaatttcttcgttaacttaatttgtcaaatcaggctcaaagatcgtttttcatatcggaatgaggattttatttattcttttaaaaataaagaggtggtgagctaaacaatttcaaatctcatcacacatatggggtcagccacaaaaataaagaacggttggaacggatctattatataatactaataaagaatagaaaaaggaaagtgagtgaggtgcgagggagagaatttgtttaatatcaaattcggctctgacttcaactgagtttaggcaattctgataaggcggggaaaatcatagtgctgagtctagaaaaatctcatgtagtgtgagataaacctcaacaaggacggtgagtccatagaggggtgtatgtgacaccccaacctcaacaaaagaaaacctcctttcaacaaggacattgggtccatagaggggatgtatgtgacatcataacttaagataaaaatgtaagaattattattaattatgtcttacttatggcccaagtttacttgtaacccaagtaataccataaataatatttaataaggaatagatctcgtccgtacattggttacttgatggacgtaccggattaagtcataacctgtataaattggtcctccctccacccattagggttaccacatattctctacaataattccatcgctgacggttgtggtaacataaagttagggaaaggaggtagaaaccaatttacgaccaacgcttccgcttctctctgtgatgatgtcttcctcatcaggtatgtgcccctaacgatctctatgtaagattatcgtgttcaagatcctgatattatgtattaaaacgtatttaatcttacatatggtagattgtttgagcggataatcttcatatacctcccctcaacaaggacatttgcggtgctaaacaatttcaagtctcatcacacatatatattatataatactaataaagaatagaaaaaggaaaggtaaacatcactaatgacagttgcggtgcaaagtgagtgaggtaataaacatcactaacttttattggttatgtcaaactcaaaataaaatttctcaacttgtttacgtgcctatatataccatgcttgttatatgctcaaagcgctcatcacttcttatagccattttgcctcctttcacttctcacctttatcgacaacaccaacaatggcggctgctgcctcaccatctccatgtttctccaaaaccctacctccatcttcctccaaatcttccaccattcttcctagatctaccttccctttccacaatcaccctcaaaaagcctcaccccttcatctcacccacacccatcatcatcgtcgtggtttcgccgtttccaatgtcgtcatatccactaccacccataacgacgtttctgaacctgaaacattcgtttcccgtttcgcccctgacgaacccagaaagggttgtgatgttcttgtggaggcacttgaaagggagggggttacggatgtatttgcgtacccaggaggtgcttctatggagattcatcaggctttgacacgttcgaatattattcgtaatgtgctgccacgtcatgagcaaggtggtgtgtttgctgcagagggttacgcacgggcgactgggttccctggtgtttgcattgctacctctggtccgggagctacgaatcttgttagtggtcttgcggatgctttgttggatagtattccgattgttgctattacgggtcaagtgtcgaggaggatgattggtactgatgcgtttcaggaaacgcctattgttgaggtaacgagatctattacgaagcataattatcttgttatggatgtagaggatattcctagggttgttcgtgaagcgttttttctagcgaaatcgggacggcctgggccggttttgattgatgtacctaaggatattcagcaacaattggtgatacctaattgggatcagccaatgaggttgcctggttacatgtctaggttacctaaattgcctaatgagatgcttttggaacaaattattaggctgatttcggagtcgaagaagcctgttttgtatgtgggtggtgggtgtttgcaatcaagtgaggagctgagacgatttgtggagcttacgggtattcctgtggcgagtactttgatgggtcttggagcttttccaactggggatgagctttcccttcaaatgttgggtatgcatgggactgtgtatgctaattatgctgtggatggtagtgatttgttgcttgcatttggggtgaggtttgatgatcgagttactggtaaattggaagcttttgctagccgagcgaaaattgtccacattgatattgattcggctgagattggaaagaacaagcaacctcatgtttccatttgtgcagatatcaagttggcattacagggtttgaattccatattggagggtaaagaaggtaagctgaagttggacttttctgcttggagacaggagttaacggaacagaaggtgaagtacccattgagttttaagacttttggtgaagccatccctccacaatatgctattcaggttcttgatgagttaactaacggaaatgccattattagtactggtgtggggcaacaccagatgtgggctgcccaatactataagtacaaaaagccacaccaatggttgacatctggtggattaggagcaatgggatttggtttgcctgctgcaataggtgcggctgttggaagaccgggtgagattgtggttgacattgatggtgacgggagttttatcatgaatgtgcaggagttagcaacaattaaggtggagaatctcccagttaagattatgttgctgaataatcaacacttgggaatggtggttcaaagggaggatcgattctataaggctaacagagcacacacttacttgggtgatcctgctaatgaggaagagatcttccctaatatgttgaaattcgcagaggcttgtggcgtacctgctgcaagagtgtcacacagggatgatcttagagctgccattcaaaagatgttagacactcctgggccatacttgttggatgtgattgtacctcatcaggagcacgttctacctatgattcccagtggcggtgctttcaaagatgtgatcacagagggtgatgggagacgttcatattgacttttagaaactacataactagctctaggcattgtattatctaaaataaacttctattaagccaaaagtgttctatctgtctagtttaaactccgagcaaacaaagcttcttattaattcaaggtctcgggttgtagtagtaaccttactatgctaaataataaacgcttgcaatatttatgattgcacgcatttaagtatttcaacctcaaaataaaaggagtttgagggataaatttcaatagaaatgtctctctatgtaatgtgtgcttggattatgtaaccttttggttgtgttaaatatttaaataaattatcgttattttatgctatggctatttgaatcttcttttcaaagcaaaaagaaaaaaaatataatttgaagtgccaagttaatattatacaaataattatgatatgatataagagattcttcaactttagatatcctaaatgaatgagaaaaaaatagtactactttgaggcaagtgatttgatccatcagtatcaaggtaaattaacaatataatgta

SEQ ID NO: 32 - DNA, 655 bp, Artificial Sequence
  Synthetic vector
gatatcagttgaatggaggtctagagtcacctgcaactgttgcatcagaatcaggtccatcgaggaaaactccactcaaggctagttcgagaagtaggagatggtcccctattgcgagaagaacaacatgctcacttttacaaccacatcctagtaatgacaaggaaggcattacagagaaactctggaaaggctgggggttaacaaaagaacggcaaaacagtcgaagacctcgttctatcttctcttttagctgattagttatttttagagccagtattttggacttatgaaaatattatatggtgcacaaagttgattcataggagtagtttatttttgaggcatttctgctcttggagattgtatgtacaggtagtaacccgaattcgtatagcatacattatacgaaggcatctgctggtcagccacttgatttgttgatgcttcaacctgctcgatatgtccttgagggaaatagtacacgagctcaccctctctaggtactgtcacaagtggaccggcacatgaacgccaaagctccgtgtacaacgccgtatcagcatcaactcttccgacaccggaactactcctaacagtatctgaaaccggcctagaaccatcactcggctcgctgtaaccttgaatcgatatc

SEQ ID NO: 33 - DNA, 34 bp, Artificial Sequence
  Solanum tuberosum
ataacttcgtatagcatacattatacgaagttat

SEQ ID NO: 34 - DNA, 34 bp, Artificial Sequence
  Solanum tuberosum
ccgaattcgtatagcatacattatacgaaggcat

SEQ ID NO: 35 - DNA, 2316 bp, Artificial Sequence
  Synthetic vector
gtcgacagtaaaagttgcacctggaataaggttttcattcttcacaggaggcatctcactctttctagcaggtcttgaacgcttagattgaacagatgtaggactcacatctgatatggaggattcttgacttgtttcagcagcatcagatgaagcttctgagacttcacctgatccatcatctgtagcagttgcttctacttcttccactgctacatcagtctcagttgctgatactataagacctcttaatttaggtcgtaaaatgcaaccaactctaaaatggggaaacaatttaatagatgttgacagaggcaggatatattttggggtaaacgggaattcttcagcagttgctcgagggagattggcggtgctttcagctcaccttgcagcttcactcaacgtctccgatttaacaaccttcaaacttctagagtcacctgcaactgttgcatcagaatcaggtccatcgaggaaaactccactcaaggctagttcgagaagtaggagatggtcccctattgcgagaagaacaacatgctcacttttacaaccacatcctagtaatgacaaggaaggcattacagagaaactctggaaaggctgggggttaacaaaagaacggcaaaacagtcgaagacctcgttctatcttctcttttagctgattagttatttttagagccagtattttggacttatgaaaatattatatggtgcacaaagttgattcataggagtagtttatttttgaggcatttctgctcttggagattgtatgtacaggtagtaacccgaattcgtatagcatacattatacgaaggcatctgctggtcagccacttgatttgttgatgcttcaacctgctcgatatgtccttgagggaaatagtacacgagctcaccctctctaggtactgtcacaagtggaccggcacatgaacgccaaagctccgtgtacaacgccgtatcagcatcaactcttccgacaccggaactactcctaacagtatctgaaaccggcctagaaccatcactcggctcgctgtaaccttgaatcgatgagcggaccggtaagaagtatccggttcaggtttctgaggatggcactatcaaagccaccgacttaaagaagataacaacaggacagaatgataaaggtcttaagctttatgatccaggctatctcaacacagcacctgttaggtcatcaatatgctatatagatggtgatgccgggatccttagatatcagttgaatggaggtctagagtcacctgcaactgttgcatcagaatcaggtccatcgaggaaaactccactcaaggctagttcgagaagtaggagatggtcccctattgcgagaagaacaacatgctcacttttacaaccacatcctagtaatgacaaggaaggcattacagagaaactctggaaaggctgggggttaacaaaagaacggcaaaacagtcgaagacctcgttctatcttctcttttagctgattagttatttttagagccagtattttggacttatgaaaatattatatggtgcacaaagttgattcataggagtagtttatttttgaggcatttctgctcttggagattgtatgtacaggtagtaacccgaattcgtatagcatacattatacgaaggcatctgctggtcagccacttgatttgttgatgcttcaacctgctcgatatgtccttgagggaaatagtacacgagctcaccctctctaggtactgtcacaagtggaccggcacatgaacgccaaagctccgtgtacaacgccgtatcagcatcaactcttccgacaccggaactactcctaacagtatctgaaaccggcctagaaccatcactcggctcgctgtaaccttgaatcgatatcatacagtcaatgccccatgatgctcatccaatgggggttcttgtcagtgcaatgagtgctctttccgtttttcatcctgatgcaaatccagctctgagaggacaggatatatacaagtgtaaacaatttaaaagcatatggtggcactgctcaatatatgaggtgggcgcgagaagcaggtaccaatgtgtcctcatcaagagatgcattctttaccaatccaacggtcaaagcatactacaagtcttttgtcaaggctattgtgacaagaaaaaactctataagtggagttaaatattcagaagagcccgccatatttgcgtgggaactcataaatgagcctcgttgtgaatccagttcatcagctgctgctctccaggcgtggatagcagagatggctggatttgtcgac

SEQ ID NO: 36 - DNA, 1480 bp, Artificial Sequence
  Synthetic vector
gtcgacagtaaaagttgcacctggaataaggttttcattcttcacaggaggcatctcactctttctagcaggtcttgaacgcttagattgaacagatgtaggactcacatctgatatggaggattcttgacttgtttcagcagcatcagatgaagcttctgagacttcacctgatccatcatctgtagcagttgcttctacttcttccactgctacatcagtctcagttgctgatactataagacctcttaatttaggtcgtaaaatgcaaccaactctaaaatggggaaacaatttaatagatgttgacagaggcaggatatattttggggtaaacgggaattcttcagcagttgctcgagggagattggcggtgctttcagctcaccttgcagcttcactcaacgtctccgatttaacaaccttcaaacttctagagtcacctgcaactgttgcatcagaatcaggtccatcgaggaaaactccactcaaggctagttcgagaagtaggagatggtcccctattgcgagaagaacaacatgctcacttttacaaccacatcctagtaatgacaaggaaggcattacagagaaactctggaaaggctgggggttaacaaaagaacggcaaaacagtcgaagacctcgttctatcttctcttttagctgattagttatttttagagccagtattttggacttatgaaaatattatatggtgcacaaagttgattcataggagtagtttatttttgaggcatttctgctcttggagattgtatgtacaggtagtaacccgaattcgtatagcatacattatacgaaggcatctgctggtcagccacttgatttgttgatgcttcaacctgctcgatatgtccttgagggaaatagtacacgagctcaccctctctaggtactgtcacaagtggaccggcacatgaacgccaaagctccgtgtacaacgccgtatcagcatcaactcttccgacaccggaactactcctaacagtatctgaaaccggcctagaaccatcactcggctcgctgtaaccttgaatcgatatcatacagtcaatgccccatgatgctcatccaatgggggttcttgtcagtgcaatgagtgctctttccgtttttcatcctgatgcaaatccagctctgagaggacaggatatatacaagtgtaaacaatttaaaagcatatggtggcactgctcaatatatgaggtgggcgcgagaagcaggtaccaatgtgtcctcatcaagagatgcattctttaccaatccaacggtcaaagcatactacaagtcttttgtcaaggctattgtgacaagaaaaaactctataagtggagttaaatattcagaagagcccgccatatttgcgtgggaactcataaatgagcctcgttgtgaatccagttcatcagctgctgctctccaggcgtggatagcagagatggctggatttgtcgac

SEQ ID NO: 37 - DNA, 34 bp, Artificial Sequence
  Synthetic vector
ataacttcgtataatgtatgctatacgaagttat

SEQ ID NO: 38 - DNA, 34 bp, Artificial Sequence
  Synthetic vector
atgacttcgtataatgtatgctatacgaagtgtg

SEQ ID NO: 39 - DNA, 34 bp, Artificial Sequence; identical to SEQ ID NO: 37
  Synthetic vector

SEQ ID NO: 40 - DNA, 34 bp, Artificial Sequence
  Synthetic vector
ataccttcgtataatgtatgctatacaaagaaat

SEQ ID NO: 41 - DNA, 34 bp, Artificial Sequence; identical to SEQ ID NO: 37
  Synthetic vector

SEQ ID NO: 42 - DNA, 34 bp, Artificial Sequence
  Synthetic vector
gccactccgtataatgtatgctatacgaaatgat

SEQ ID NO: 43 - DNA, 185 bp, Artificial Sequence
  Synthetic vector
cttaagaaattttggaattcttagcccttctgttcctatactttctagagaataggaagttgctaacttctcctgattgtccgatataacctgtcctatagtttgtgtttagcatttcattatggtattccactttggtaatcatggtcatttttcattgaaaagcaacctaggtttgtggatcc

SEQ ID NO: 44 - DNA, 185 bp, Artificial Sequence; identical to SEQ ID NO: 43
  Synthetic vector

SEQ ID NO: 45 - DNA, 34 bp, Artificial Sequence
  Solanum tuberosum
tctgttcctatactttctagagaataggaagttg

SEQ ID NO: 46 - DNA, 1432 bp, Artificial Sequence
  Synthetic vector
gtcgacagtaaaagttgcacctggaataaggttttcattcttcacaggaggcatctcactctttctagcaggtcttgaacgcttagattgaacagatgtaggactcacatctgatatggaggattcttgacttgtttcagcagcatcagatgaagcttctgagacttcacctgatccatcatctgtagcagttgcttctacttcttccactgctacatcagtctcagttgctgatactataagacctcttaatttaggtcgtaaaatgcaaccaactctaaaatggggaaacaatttaatagatgttgacagaggcaggatatattttggggtaaacgggaattcttagcccttctgttcctatactttctagagaataggaagttgctaacttctcctgattgtccgatataacctgtcctatagtttgtgtttagcatttcattatggtattccactttggtaatcatggtcatttttcattgaaaagcaacctagaaacttccggtgtatccgccgtttccggcgttgcacctccgccgaatctaaaaggtgcgttgacgatcatcgatgagcggaccggtaagaagtatccggttcaggtttctgaggatggcactatcaaagccaccgacttaaagaagataacaacaggacagaatgataaaggtcttaagaaattttggaattcttagcccttctgttcctatactttctagagaataggaagttgctaacttctcctgattgtccgatataacctgtcctatagtttgtgtttagcatttcattatggtattccactttggtaatcatggtcatttttcattgaaaagcaacctaggtttgtggatccttagatatcgaggctaccctattgaagagctggccgagggaagttccttcttggaagtggcatatcttttgttgtatggtaatttaccatctgagaaccagttagcagactgggagttcacagtttcacagcattcagcggttccacaaggactcttggatatcatacagtcaatgccccatgatgctcatccaatgggggttcttgtcagtgcaatgagtgctctttccgtttttcatcctgatgcaaatccagctctgagaggacaggatatatacaagtgtaaacaatttaaaagcatatggtggcactgctcaatatatgaggtgggcgcgagaagcaggtaccaatgtgtcctcatcaagagatgcattctttaccaatccaacggtcaaagcatactacaagtcttttgtcaaggctattgtgacaagaaaaaactctataagtggagttaaatattcagaagagcccgccatatttgcgtgggaactcataaatgagcctcgttgtgaatccagttcatcagctgctgctctccaggcgtggatagcagagatggctggatttgtcgac

SEQ ID NO: 47 - DNA, 685 bp, Artificial Sequence
  Synthetic vector
tttctagcaagtcttgtacgcttagattgaacagatgtaggactcacatctgatatggaggattcttgacttgtttcagcagcatcagatgaagcttctgagacttcacctgatccatcatctgtagcagttgcttctacttcttccactgctacatcagtctcagttgctgatactataagacctcttaatttaggtcgtaaaatgcaaccaactctaaaatggggaaacaatttaatagatgttgacagaggcaggatatattttggggtaaacgggaatnnntagcccttctgttcctatactttctagagaataggaagttgctaacttctcctgattgtccgatataacctgtcctatagtttgtgtttagcatttcattatggtattccactttggnaatcatggtcanttttcattgaaaagcaacctaggtttgtggatccttagatatcgaggctaccctattgaagagctggccgagggaagttccttcttggaagtggcatatcttttgttgtatggtaatttaccatctgagaaccagttagcagactgggagttcacagtttcacagcattcagcggttccacaaggactcttggatatcatacagtcaatgccccatgatgctcatccaatgggggtacttgtcagtgcaatgagtgctctttccgttttt

SEQ ID NO: 48 - DNA, 875 bp, Artificial Sequence
  Synthetic vector
attaatcccacctgcaggatcaacattctccacaatgatggcatgccttctactaggaacactaggagcatccatcactgaagtagtaaccgatgctcttgtagctgagtacagcaaaactcaaaaagcaggcgttctacagtcgtatgccttcttagcacttgcagcaggtgcattgctaggcaacttatcgggcgggtttttcctccaaatcacccaggacccaaaatccatgttcctcgctttctccgttctcctcactgcacagctagctctttccttaaaaaccaaagagatcgctcttccttcatcaaattctacccgggcctccttatccaaaaacctcaaaagacagctttctgaactagtaactgctatcaaagaaccaagaattttctatcctcttctctggattgtagcttctacagcacttgttcctatactctctggagaataggaactgtatatcatcctctttcacttgaataggagatggagataagggagtatgcttgcaaatgacaaaagcaagtagtacatataggtgattttggattctagttgacatcgactatgagtaccttcctgggacaactgggaaggaagatggcaatagtttataaggcttaatcacaattaaggcttcacatacctaagtctaattcgcttgaccgtctcaatatcaaaacaaaccaactctccaatcaaattttcaaccaattaattccaatcatagactgtagaacagacgaaattttagagaaatggacccaaatagagatagaaagtactactcacgagaagagtcgatggaggggctccaaagagtaataattaaaggttagccttcaataatcatgggtttagcctctaga

SEQ ID NO: 49 - DNA, 34 bp, Artificial Sequence
  Allium cepa
gaagttcctatactttctagagaataggaacttc

SEQ ID NO: 50 - DNA, 34 bp, Artificial Sequence
  Synthetic vector
cttgttcctatactctctggagaataggaactgt

SEQ ID NO: 51 - DNA, 2896 bp, Artificial Sequence
  Synthetic vector
gtcgacttccctttcctctactccacttgtttctcgctttctctacttcctttttctctcttttctttatatttattgctcagctgggattaattactgtcatttattcctcatatctattttattgaattaaaacggttatttagctcgaggccttctctcttattctttgcttccaaggagagagaatatggcgagtggtagcaatcatcagcatggtggaggaggaagaagaagaggcggaatgttagtcgctgcgaccttgcttattcttcctgccattttccccaatttgtttgttcctcttccctttgcttttggtagttctggcagcggtgcatctccttctctcttctccgaatggaatgctcctaaacctaggcatctctctcttctgaaagcagccattgagcgtgagatttctgacgaacaaaaatcagagctgtggtctcccttgcctccacagggatggaaaccgtgccttgagactcaatatagtagcgggctacccagtagatcgacaggatatattcaagtgtaaaacaagatgctgaatcgattagcaatggttcgctcttctagctctttccttaaaaaccaaagagatcgctcttccttcatcaaattctacccgggcctccttatccaaaaacctcaaaagacagctttctgaactagtaactgctatcaaagaaccaagaattttctatcctcttctctggattgtagcttctacagcacttgttcctatactctctggagaataggaactgtatatcatcctctttcacttgaataggagatggagataagggagtatgcttgcaaatgacaaaagcaagtagtacatataggtgattttggattctagttgacatcgactatgagtaccttcctgggacaactgggaaggaagatggcaatagtttataaggcttaatcacaattaaggcttcacatacctaagtctaattcgcttgaccgtctcaatatcaaaacaaaccaactctccaatcaaattttcaaccaattaattccaatcatagactgtagaacagacgaaattttagagaaatggacccaaatagagatagaaagtactactcacgagaagagtcgatggaggggctccaaagagtaataattaaaggttagccttcaataatcatgggtttagcctctagacttgcttctcggataatcaatcctcagtttttgattccttctcgaagcttccttgatctccataagatggtaaacaaggaggcgataaaaaaagaaagggctagacttgctgatgagatgagcagaggatattttgcggatatggcagagattcgtatacatggtggcaagattgctatggcaaatgaaattcttattccatcaggggaagcaatcaaatttcctgatttgacagtaaaattgtctgatgatagcagtttgcatttaccaattgtatctacacaaagtgctacaaataacaatgctaaatccactcctgctgcctcattgttgtgcctttccttcagagcaagttcacagacaatggttgaatcatggactgttccttttttggacacttttaactcttcagaagtacaagcataatcccacctgcaggatcaacattctccacaatgatggcatgccttctactaggaacactaggagcatccatcactgaagtagtaaccgatgctcttgtagctgagtacagcaaaactcaaaaagcaggcgttctacagtcgtatgccttcttagcacttgcagcaggtgcattgctaggcaacttatcgggcgggtttttcctccaaatcacccaggacccaaaatccatgttcctcgctttctccgttctcctcactgcacagctagctctttccttaaaaaccaaagagatcgctcttccttcatcaaattctacccgggcctccttatccaaaaacctcaaaagacagctttctgaactagtaactgctatcaaagaaccaagaattttctatcctcttctctggattgtagcttctacagcacttgttcctatactctctggagaataggaactgtatatcatcctctttcacttgaataggagatggagataagggagtatgcttgcaaatgacaaaagcaagtagtacatataggtgattttggattctagttgacatcgactatgagtaccttcctgggacaactgggaaggaagatggcaatagtttataaggcttaatcacaattaaggcttcacatacctaagtctaattcgcttgaccgtctcaatatcaaaacaaaccaactctccaatcaaattttcaaccaattatgaggtatcatttttggattcttggtttttctcattcggaccaatcaagagaatgtttcttaacatgacgaagaaacccactgctactcagcggaagattggttatttcatttggtgatcactatgattttaggaagcagcttcaaattgtaaatcttttgacaggatatatattactgtaaaaagtgaagagagaaatgtgatatatgctgatgtttccatggagaggggtgcatttcttgttcaacaagctatgagggctttccatggaaagaatatagaaagcgcaaaatcaaggcttagtctttgcgaggaggatattcgtgggcagttagagatgacagataacaaaccagagttatattcacagcttggtgctgtccttggaatgctaggagactgctgtcgaggaatgggtgatactaatggtgcgattccatattatgaagagagtgtggaattcctcttaaaaatgcctgcaaaagatcccgaggttgtacatacactatcagtttccttgaataaaattggagacctgaaatactacgaaggagatctgcagtcgac

SEQ ID NO: 52 - DNA, 34 bp, Artificial Sequence; identical to SEQ ID NO: 49
  Synthetic vector

SEQ ID NO: 53 - DNA, 34 bp, Artificial Sequence
  Synthetic vector
acagttcctatactttctggagaataggaaggtg

SEQ ID NO: 54 - DNA, 34 bp, Artificial Sequence; identical to SEQ ID NO: 49
  Synthetic vector

SEQ ID NO: 55 - DNA, 34 bp, Artificial Sequence
  Synthetic vector
acagttcctatactttctacagaataggaacttc

SEQ ID NO: 56 - DNA, 34 bp, Artificial Sequence; identical to SEQ ID NO: 49
  Synthetic vector

SEQ ID NO: 57 - DNA, 34 bp, Artificial Sequence
  Synthetic vector
agagttcctatactttctagagaataggaacccc

SEQ ID NO: 58 - DNA, 34 bp, Artificial Sequence; identical to SEQ ID NO: 49
  Synthetic vector

SEQ ID NO: 59 - DNA, 34 bp, Artificial Sequence
  Synthetic vector
aaagttcctatactttctggagaataggaaaaca

SEQ ID NO: 60 - DNA, 31 bp, Artificial Sequence
  Synthetic primer
ggggtaccatgaatacttctgtttttacgtc

SEQ ID NO: 61 - DNA, 20 bp, Artificial Sequence
  Synthetic primer
gccatcaaacaacccgataa

SEQ ID NO: 62 - DNA, 23 bp, Artificial Sequence
  Synthetic primer
tgcaatgaaattgataaaacacc

SEQ ID NO: 63 - DNA, 24 bp, Artificial Sequence
  Synthetic primer
tcatcaaaggaggacggagcaaga

SEQ ID NO: 64 - DNA, 34 bp, Artificial Sequence
  Synthetic vector
ataacttcgtatannnnnnnntatacgaagttat

SEQ ID NO: 65 - DNA, 34 bp, Artificial Sequence
  Synthetic vector
gaagttcctatacnnnnnnnngwataggaacttc

SEQ ID NO: 66 - DNA, 24 bp, Artificial Sequence
  Synthetic vector
grcaggatatatnnnnnkstmawn

SEQ ID NO: 67 - DNA, 539 bp, Petunia x hybrida
acttgtagtatcaaacgttcaattgaaatcatagttaaaagttaatcatgagagcttagctaactgttgggacacttggactgaaattttcttacttacacttttatatttttctgttctttctctaacatttgttctcattgacaattcaccacacatatgagtggttcgctagttcgatatggccatgagttgagattatatatgctttggccaagtggatattatattgcaattaatctactatcagatgtggcaaccttggatttgctgaaaacggaaaatctgcattgggttggatttcttaaaagtaatgtatctaaaaaaatatagtcatgtttaacggtgctgaatttgccaactggacaagaatgcaaatgttacacattgtcatccaccaattaggaaatagatagtgatattcaaggataaggacttagggtctttcgagtcatttaaataaacttgttggaagatccatgaaactcatcaactcttctttctgtgtaatagctgcattcaagagtttttcagttact

SEQ ID NO: 68 - protein, 262 aa, Petunia x hybrida
MNTSSTIPKSSGLVRKGAWTEEEDVLLRKCIEKFGEGKWHQVPVRAGLNRCRKSCRLRWLNYLRPHIKRGDFSEDEVDLILRLHKLLGNRWSLIAGRLPGRTANDVKNYWNTHLQRKLIAPPRQEIRKCRALKITENNIVRPRPRTFSNNAQNISWCSNKSITTSTIDKDGSNNECIRINDKKPMAEESRHDGVQWWTSLLANCNENDETAVENMSYDKLPSLLHEEISPTINGGISNCMQEGQTGWDDFSVDIDHLWNLLN

SEQ ID NO: 69 - protein, 262 aa, Petunia x hybrida
MNTSVFTSSGVLRKGAWAEEEDILLRKCIEKYGEGKWHQVPVRAGLNRCRKSCRLRWLNYLRPHIKRGDFCPEEVDLIQRLHKLLGNRWSLIAGRLPGRTANDVKNYWNTHLLRRSNFAPPPQQHERKCTKEIRTMAKNAIIRPQPRNLSKLAKNNVSNHSTKHKDEYSKQKMFIEKPTTAEVVSRDNNVEWWTNLLLDNCNGFEKAAPESSSTFKNIESLLNEELLSASINGGTNYPIQETGDMGWSDFCIDSDPWELLLQ

(19) United States
(12) Patent Application Publication
(10) Pub. No.: US 2012/0042409 A1
(43) Pub. Date: Feb. 16, 2012

(54) PLANT TRANSFORMATION USING DNA MINICIRCLES

(76) Inventors: Anthony Conner, Lincoln (NZ); Julie Pringle, Christchurch (NZ); Annemarie Lokerse, Wageningen (NL); Johanna Jacobs, Lincoln (NZ); Philippa Barrell, Rangiora (NZ); Simon Deroles, Levin (NZ); Murray Boase, Palmerston North (NZ)

(21) Appl. No.: 13/144,543

(22) PCT Filed: Jan. 15, 2010
(86) PCT No.: PCT/NZ2010/000005

§ 371 Date: Nov. 1, 2011

(30) Foreign Application Priority Data
Jan. 15, 2009 (NZ) 574191

(51) Int. Cl. A01H 1/06; C12N 15/64; A01H 5/00; C12N 15/63; C12N 5/10

(52) U.S. Cl. 800/278; 435/320.1; 435/419; 800/298; 435/91.4

(57) ABSTRACT

The invention provides methods and compositions for producing and using minicircle DNA molecules that are useful for plant transformation. The invention also provides methods for transforming plant cells and plants with such minicircle DNA molecules, plant cells and plants produced by such methods, and plants transformed with minicircle DNA molecules. The methods and compositions of the invention are particularly useful for producing “intragenic plants” which do not contain any non-native DNA.

### BACKGROUND ART

Historically, plant breeders have succeeded in introducing pest and disease resistance, as well as improved quality attributes, into a wide range of crop plants through traditional plant breeding methods. In recent years, genetic engineering has widened the scope by which new traits can be incorporated into plants at the DNA level. Such plants with extra DNA incorporated are usually referred to as transformed plants, transgenic plants or genetically modified (GM) plants.

The first definitive demonstration of the successful transformation of plants with foreign genes involved the transfer and expression of a neomycin-phosphotransferase gene from bacterial transposon five (Tn5) [Bevan et al 1983; Fraley et al 1983; Herrera-Estrella et al 1983]. The resulting plants were able to grow in the presence of aminoglycoside antibiotics (e.g. kanamycin) due to the detoxifying activity of the transgene-derived enzyme. Southern analysis established the integration of the foreign gene into the genome of plant cells, northern analysis demonstrated the expression of RNA transcripts of the correct size, and enzyme assays established the activity of neomycin-phosphotransferase in the plant cells. This demonstrated that genes of non-plant origin could be transferred to and expressed in plants greatly expanded the potential sources of genes (other plants, microbes, animals, or entirely synthetic genes) available for introduction into crop plants.

Nowadays two general approaches can be used to develop transformed plants. These involve the direct uptake of DNA into plant cells, or exploiting the natural gene transfer ability of the bacterium Agrobacterium.

### Direct DNA Uptake

Direct gene transfer involves the uptake of naked DNA by plant cells and its subsequent integration into the genome. The target cells can include: isolated protoplasts or cells; cultured tissues, organs or plants; intact pollen, seeds, and plants [Petolino 2002]. Direct DNA uptake methods are entirely physical processes with no biological interactions to introduce the DNA into plant cells and therefore no “host range” limitations associated with Agrobacterium-mediated transformation [Twyman and Christou 2004]. Methods to effect direct DNA transfer can involve a wide range of approaches, including: passive uptake; the use of electroporation; treatments with polyethylene glycol; electrophoresis; cell fusion with liposomes or spheroplasts; microinjection, silicon carbide whiskers, and particle bombardment [Petolino 2002]. Of the various approaches, particle bombardment is almost exclusively used because there are no limitations to the target tissue. However, one limitation of particle bombardment is the overall length of the DNA. Longer DNA molecules are likely to shear either upon particle acceleration or impact [Twyman and Christou 2004].

Vectors for direct DNA uptake only need to be standard bacterial plasmids to allow propagation of the vector. It is usual for such vectors to be small, high-copy plasmids capable of propagation in Escherichia coli. This allows convenient construction of plasmids using well-established molecular biology protocols and ensures high yields of vector upon plasmid isolation and purification for subsequent use in transformation. Various authors claim a preference to use DNA of a specific form (circular or linear, double- or single-stranded). However, comparisons of all four combinations of DNA conformation in parallel experiments resulted in similar transformation frequencies and integration patterns [Uze et al 1999].

### Agrobacterium-Mediated Gene Transfer

Agrobacterium strains induce crown galls or hairy roots on plants by the natural transfer of a discrete segment of DNA (T-DNA) to plant cells. The T-DNA region contains genes that induce tumour or hairy root formation and opine biosynthesis in plant cells. In Agrobacterium the T-DNA resides on the Ti or Ri plasmids along with several virulence loci with key vir genes responsible for the transfer process [Gheysen et al 1998; Gelvin 2003]. The action of these vir genes, combined with several other chromosomal-based genes in Agrobacterium, and specific plant proteins [Anand et al 2007] effect the transfer and integration of the T-DNA into the nuclear genome of plant cells. Short imperfect direct repeats of about 25 bp, known as the right and left border (RB and LB respectively), define the outer limits of the T-DNA region [Gheysen et al 1998; Gelvin 2003].

The genes on the T-DNA of Ti and Ri plasmids responsible for tumour or hairy root formation are well known to result in plants with an abnormal phenotype or prevent the regeneration of plants [Grant et al 1991; Christey 2001]. The development of “disarmed” Agrobacterium strains with either the deletion of the genes responsible for tumour formation or the complete removal of the T-DNA was crucial for Agrobacterium-mediated gene transfer to plants. These approaches lead to the development of co-integrate vectors and binary vectors respectively.

With co-integrate vectors the foreign DNA is integrated into the resident Ti plasmid [Zambryski et al 1983]. The tumour-inducing genes of the T-DNA are first removed leaving the right border and left border sequences. The foreign DNA is then inserted into a vector that can not replicate in Agrobacterium cells, but can recombine with the Ti plasmids through a single or double recombination event at a homologous site previously introduced between the right border and left border sequences. This results in a co-integration event between the two plasmids. A later refinement resulted in the split-end vector system [Fraley et al 1985] in which only the left border is retained on the Ti plasmid and the right border is restored by the co-integration event. The main advantage of co-integrate vectors is their high stability in Agrobacterium. However, the frequency of co-integration is low and their development is complex, requiring a detailed knowledge of the Ti plasmid and a high level of technical competence.

The demonstration that the T-DNA and the vir region of Ti plasmids could be separated onto two different plasmids [Hoekema et al 1983; de Frammond et al 1983] contributed to the development of binary vectors, a key step to greatly simplify Agrobacterium-mediated gene transfer. The helper plasmid is a Ti or Ri plasmid that has the vir genes with the T-DNA region deleted and acts in trans to effect T-DNA processing and transfer to plant cells of a T-DNA on a second plasmid (the binary vector). Binary vectors have several main advantages: small size, ease of manipulation in Escherichia coli, high frequency of introduction into Agrobacterium, and independence of specific Ti and Ri plasmids [Grant et al 1991]. They have revolutionised the applications of Agrobacterium-mediated gene transfer in plant science and are now used to the virtual exclusion of co-integrate vectors.

To facilitate the development of transgenic plants a wide range of binary vectors with versatile T-DNA regions have been constructed [e.g. Hellens et al 2000]. These often contain alternative cloning regions with a different series of unique restriction endonuclease sites for insertion of genes for transfer to plants and/or alternative selectable marker genes. However, many binary vectors also contain extraneous DNA elements on the T-DNA region that are present as a matter of convenience rather than of necessity for the development of a desired transgenic plant. Examples include the lacZ′ region coding for β-galactosidase reporter genes, origins of plasmid replication, and bacterial marker genes.

For the general release of transgenic plants into agricultural production, such extraneous DNA regions either necessitate additional risk assessment or may be unacceptable to regulatory authorities [Nap et al 2003]. This led to the development of minimal T-DNA vectors, without extraneous DNA segments on the T-DNA [During 1994; Porsch et al 1998; Barrell et al 2002; Barrell and Conner 2006]. These simple binary vectors consist of a very small T-DNA with a selectable marker gene tightly inserted between the left and right T-DNA borders and a short cloning region with a series of unique restriction sites for inserting genes-of-interest. As a consequence they are based on the minimum features necessary for efficient plant transformation by Agrobacterium.

For optimal transgene function, the generation of plants with a single intact T-DNA is preferred. The T-DNA is delineated by two 25 bp imperfect repeats, the so-called border sequences, which define target sites for the VirD1/VirD2 border specific endonucleases that initiate T-DNA processing [Gelvin 2003]. The resulting single-stranded T-strand is transferred to plant cells rather than the double stranded T-DNA. Initiation of T-strand formation involves a single strand nick in the double-stranded T-DNA of the right border, predominantly between the third and fourth nucleotides. After nicking the border, the VirD protein remains covalently linked to the 5′ end of the resulting single-stranded T-strand [Gheysen et al 1998; Gelvin 2003]. The attachment of the VirD protein to the 5′ right border end of the T-strand, rather than the border sequence, establishes the polarity between the borders. This determines the initiation and termination sites for T-strand formation.

Vectors for Agrobacterium-mediated transformation of plants generally contain two T-DNA border-like sequences in the correct orientation that ideally flank a series of restriction sites suitable for cloning genes intended for transfer. However, efficient transformation is possible with, only a single border in the right border orientation. Deletion of the left border has minimal effect on T-DNA transfer, whereas deletion of the right border abolishes T-DNA transfer [Gheysen et al 1998], Retaining two borders flanking the T-DNA helps to define both the initiation and end points of transfer, thereby facilitating the recovery of transformation events without vector backbone sequences.

The well defined nature of T-strand initiation from the right border results, in most instances, in only 3 nucleotides of the right border being transferred upon plant transformation. However, at the left border, the end point of the T-DNA sequence is far less precise. It may occur at or about the left border, or even well beyond the left border. This is confirmed by DNA sequencing across the junctions of T-DNA integration events into plant genomes [Gheysen et al 1998]. The less precise end points at left border junctions results in the frequent integration of vector backbone sequences into plant genomes [Gelvin 2003].

### Intragenic DNA Transfers

Despite the rapid global adoption of GM technology in agricultural crops, many concerns have been raised about the use of GM crops in agricultural production [Conner et al 2003; Nap et al 2003]. These include ethical, religious and/or other concerns among the general public, with the main underlying issue often involving the transfer of genes across very wide taxonomic boundaries [Conner 2000; Conner and Jacobs 2006]. Current advances in plant genomics are beginning to address some of these concerns. Many genes are now being identified from within the gene pools already used by plant breeders for transfer via plant transformation. More importantly, the design of vectors for plant transformation has recently progressed to the development of intragenic systems [Conner et al 2005, Conner et al 2007]. This involves identifying plant-derived DNA sequences similar to important vector components. A particularly useful approach involves adjoining two fragments from plant genomes to form sequences that have the functional equivalence of vectors elements such as: T-DNA borders for Agrobacterium-mediated transformation, bacterial origins of replication, and bacterial selectable elements. Such DNA fragments have been identified from a wide range of plant species, suggesting that intragenic vectors can be constructed from the genome of any plant species [Conner et al 2005]. Intragenic vectors provide a mechanism for the well-defined genetic improvement of plants with the entire DNA destined for transfer originating from within the gene pool already available to plant breeders. The aim of such approaches is to design vectors capable of effecting gene transfer without the introduction of foreign DNA upon plant transformation. In this manner genes can be introgressed into elite cultivars in a single step without linkage drag and, most importantly, without the incorporation of foreign DNA [Conner et al 2007].

### The Problem of Vector Backbone Sequences

A major limitation of current technology to generate transformed plants, whether they involve transgenic or intragenic approaches is the inadvertent transfer of unintended DNA sequences to the transformed plants. This applies for both direct DNA uptake into plant cells and Agrobacterium-mediated gene transfer. In both instances the transfer of the vector backbone sequences is undesired. This is especially an issue when attempting intragenic transfers, as these vector backbone sequences are usually based on foreign DNA derived from bacteria. For the general release of transgenic plants into agricultural production, such extraneous DNA regions either necessitate additional risk assessment or may be unacceptable to regulatory authorities [Nap et al 2003].

For direct DNA uptake the avoidance of undesirable plasmid backbone sequences can be potentially achieved by one of several approaches:
1. Generating the desired DNA fragment via the polymerase chain reaction (PCR), thereby limiting the boundaries of the DNA to be transferred by the design of specific primers [Yang et al 2008]. However, this approach can inadvertently introduce random mutations through PCR errors, thereby resulting in the generation of non-functional or undesirable DNA fragments with unknown errors in DNA sequence.
2. The gel isolation and purification of the desired DNA fragments from plasmid propagated in bacteria. However, this is very time consuming and generally requires the use of DNA-binding chemicals to visualise DNA bands following gel electrophoresis. Such DNA-binding chemicals may induce undesired mutations in the DNA fragment.
3. Transposition-based transformation from plasmid DNA introduced into plant cells [Houba-Herin et al 1994] or from viral vectors [Sugimoto et al 1994]. However transformation frequencies are generally very low.
4. In the case of intragenic transfers, an alternative approach involves using plant-derived sequences that have the functional equivalence of bacterial origins of replication and bacterial selectable elements [Conner et al 2005].

During Agrobacterium-mediated gene transfer, vector backbone sequences beyond the left T-DNA border often integrate into plant genomes [Gelvin 2003]. The frequency of such events in transformed plants can be as high as 50% [de Buck et al 2006], 75% [Kononov et al 1997], or even 90% [Heeres et al 2002], and in some instance can involve the entire binary vector [Wenck et al 1997]. These vector backbone sequences may integrate as a consequence of either the initiation of T-strand formation from the left border or from ‘skipping’ or ‘read-through’ at the left border. The integration of vector backbone sequences into transformed plants is considered an unavoidable consequence of the mechanism of Agrobacterium-mediated gene transfer [Gelvin 2003]. However, several strategies have been proposed to either limit such transfers or to help identify plants containing such DNA:
1. Incorporating a barnase suicide gene into the vector backbone to prevent the recovery of plants expressing this gene can reduce the frequency of transformed plants with unwanted vector backbone sequences [Hanson et al 1999]. Negative selection markers such as the cytosine deaminase (codA) gene [Stougaard 1993] could also accomplish the same result. Similarly, the use of a reporter gene, such as β-glucuronidase, on the vector backbone allows the convenient recognition of plants in which vector backbone sequences have been integrated [Kuraya et al 2004]. An alternative approach involves using an isopentenyl transferase gene for cytokinin production that results in the regeneration of shoots with an easily recognisable stunted, pale green phenotype that fail to initiate roots [Rommens et al 2004]. However, in all these instances the transfer of these complete and intact genes is required to allow this strategy to be effective. The partial transfer of these genes does not allow their detection and still results in vector backbone sequences being transferred.
2. The use of multiple left borders in tandem repeats is reported to enhance the opportunity for T-strand formation to terminate at the left border region [Kuraya et al 2004]. However, this can also increase the frequency of initiation of T-strand formation at the left border resulting in co-transformation of vector backbone sequences along with the intended T-DNA regions.
3. Transposition-based transformation from the double-stranded form of T-strands following their Agrobacterium-mediated delivery into plant cells [Yan and Rommens 2007]. However, transformation frequencies were low and unanticipated transfer of other DNA regions on the T-DNA was often observed.
4. In the case of intragenic transfers, an alternative approach involves using plant-derived sequences that have the functional equivalence of bacterial origins of replication and bacterial selectable elements, thereby constructing the whole binary vector from plant genomes [Conner et al 2005].

It is an object of the invention to provide improved compositions and methods for plant transformation which reduce or eliminate the transfer of vector backbone sequences and/or foreign DNA into the plant, or at least provide the public with a useful choice.

### SUMMARY OF INVENTION

The invention provides methods and compositions for producing transformed plants by transformation using minicircle DNA molecules. The invention also provides plants, plant parts, plant progeny and plant products of plants transformed with the minicircle DNA molecules. The invention also provides compositions and methods for the production of minicircle DNA molecules. Methods and compositions are provided for both direct and Agrobacterium-based transformation. Preferably the transformed plants are free from vector backbone sequence and elements not required within the plant, such as bacterial origins of replication and selectable markers for bacteria.

Preferably the minicircles are composed entirely of plant-derived sequences. Preferably the sequences are derived from plant species that are interfertile with the plant to be transformed. More preferably the sequences are derived from the same species of plant as the plant to be transformed. In this way transformed plants can be produced that are free from non-plant or non-native DNA.

### Minicircles

Minicircles are supercoiled DNA molecules devoid of plasmid backbone sequences. They can be generated in vivo from bacterial plasmids, or vectors, by site-specific intramolecular recombination to result in minicircle DNA vectors devoid of bacterial plasmid/vector backbone DNA [Darquet et al 1997, 1999]. By the correct positioning of the sequences for site-specific recombination, the induced expression of the appropriate recombinase enzyme results in the formation of two circular DNA molecules; one (the minicircle) containing element desired to be transformed such as an expression cassette, and the other carrying the remainder of the bacterial plasmid with the origin of replication and the bacterial selectable marker gene [Chen et al 2005].

Previous work in plants using recombinase recognition sequences has focused on use of such sequences to flank undesirable elements such as foreign selectable marker sequences that are incorporated into plant genomes to allow for selection of transformants. Expression of an appropriate recombinase in such plants can effectively excise the undesirable elements from the plant genome.

In contrast the applicants' invention involves recombinase-driven production of DNA minicircles for use in plant transformation and offers a solution for the inadvertent transfer of unintended DNA sequences during plant transformation. Using this approach the applicants have shown that the transfer of bacterial replication origins, bacterial selectable marker genes and other vector backbone sequences can be prevented from transfer to plant genomes during transformation. The invention also provides compositions and methods for producing DNA minicircles containing only the DNA intended for plant transformation by utilizing plant-derived recombinase sites. By producing minicircles including only plant-derived DNA sequences the invention also provides an important tool for the effective intragenic delivery of genes by transformation without the transfer of foreign DNA. The application of minicircles for plant transformation is exemplified using both direct DNA uptake and Agrobacterium-mediated gene transfer.

### 1. Vector for Producing Plant-Derived Minicircle (Useful for Direct or Agrobacterium Intragenic Transformation)

In one aspect the invention provides a vector comprising first and second recombinase recognition sequences, wherein the recombinase recognition sequences, and any sequence between the recombinase recognition sequences, are derived from plant species.

In one embodiment the first recombinase recognition sequence and the second recombinase recognition sequence are loxP-like sequences derived from a plant species.

In an alternative embodiment the first recombinase recognition sequence and the second recombinase recognition sequences are frt-like sequences derived from plant species.

In a preferred embodiment the vector is capable of producing a minicircle DNA molecule in the presence of a suitable recombinase.

Preferably when the recombinase sites are loxP-like sequences, the recombinase is Cre.

Preferably when the recombinase sites are frt-like sequences, the recombinase is a FLP.

Preferably the minicircle produced is composed entirely of plant-derived sequence.

Preferably between the recombinase recognition sequences, the vector comprises an expression construct.

The expression construct preferably comprises a promoter and a sequence to be expressed.

In one embodiment the promoter is operably linked to the sequence to be expressed.

In an alternative embodiment, the promoter and sequence to be expressed and separated, with one of the recombinase recognition sequences between the promoter and sequence to be expressed. In this embodiment the promoter and sequence to be expressed become operably linked upon site specific recombination.

In one embodiment the promoter is a light-regulated promoter.

In one embodiment the promoter is the promoter of a chlorophyll a/b binding protein (cab) gene.

In one embodiment the promoter comprises a sequence with at least 70% identity to the sequence of SEQ ID NO:67.

In one embodiment the promoter comprises the sequence of SEQ ID NO:67.

Preferably the expression construct also comprises a terminator operably linked to the sequence to be expressed.

The sequence to be expressed may be the coding sequence encoding a polypeptide.

In one embodiment the polypeptide is an R2R3 MYB transcription factor, capable of regulating the production of anthocyanin in a plant.

In a further embodiment the polypeptide comprises a sequence with at least 70% identity to SEQ ID NO: 68 or 69.

In a further embodiment the polypeptide comprises a sequence with at least 70% identity to SEQ ID NO: 68.

In a further embodiment the polypeptide comprises a sequence with at least 70% identity to SEQ ID NO: 69.

In a further embodiment the polypeptide comprises the sequence of SEQ ID NO: 68.

In a further embodiment the polypeptide comprises the sequence of SEQ ID NO: 69.

Alternatively the sequence to be expressed may be a sequence suitable for effecting the silencing of at least one endogenous polynucleotide of polypeptide in a plant transformed with the expression construct.

The expression construct may also be an intact gene, such as a gene isolated from a plant. The intact gene may comprise a promoter, a coding sequence optionally including introns, and a terminator.

In a preferred embodiment the expression construct and the elements (promoter, sequence to be expressed, and terminator) within it are derived from plants. More preferably the expression construct and the elements within it are derived from a species interfertile with the plant species from which the recombinase recognition sequences are derived. Most preferably, the expression construct and the elements within it are derived from the same species as the plant species from which the recombinase recognition sequences are derived.

The vector may also comprise a selectable marker sequence between the recombinase recognition sequences. Preferably the selectable marker sequence is derived from a plant species. More preferably the selectable marker sequence is derived from a species interfertile with the plant species from which the recombinase recognition sequences are derived. Most preferably, the selectable marker sequence is derived from the same species as the plant species from which the recombinase recognition sequences are derived.

### 2. Vector for Producing Plant-Derived Minicircle (Useful for Agrobacterium-Mediated Intragenic Transformation)

In a further embodiment the vector comprises, between the recombinase recognition sequences, at least one T-DNA border-like sequence.

In a further embodiment the vector comprises, between the recombinase recognition sequences, two T-DNA border-like sequences.

Preferably the T-DNA border-like sequence or sequences is/are derived from a species interfertile with the plant species from which the recombinase recognition sequences are derived. More preferably, the T-DNA border-like sequence or sequences is/are derived from the same species as the plant species from which the recombinase recognition sequences are derived.

In a preferred embodiment, all of the sequences of the recombinase recognition sequences and the sequences, between the recombinase recognition sequences are derived from plant species, more preferably interfertile plant species, most preferably the same plant species.

### 3. Vector for Producing Minicircle (Useful for Agrobacterium-Mediated Transformation)

In one aspect the invention provides a vector comprising first and second recombinase recognition sequences, comprising at least one T-DNA border sequence between the recombinase recognition sequences.

In a further embodiment the vector comprises, two T-DNA border sequences between the recombinase recognition sequences.

Preferably the vector comprises one T-DNA border sequences between the recombinase recognition sequences.

In one embodiment the first recombinase recognition sequence and the second recombinase recognition sequence are loxP sequences.

In an alternative embodiment the first recombinase recognition sequence and the second recombinase recognition sequences are frt sequences.

Preferably any sequences between the recombinase recognition sequences, are derived from plant species.

In a preferred embodiment the vector is capable of producing a minicircle DNA molecule in the presence of a suitable recombinase.

Preferably when the recombinase sites are loxP sequences, the recombinase is Cre.

Preferably when the recombinase sites are frt sequences, the recombinase is a FLP.

Preferably between the recombinase recognition sequences, the vector comprises an expression construct.

The expression construct preferably comprises a promoter, and a sequence to be expressed.

In one embodiment the promoter is operably linked to the sequence to be expressed.

In an alternative embodiment, the promoter and sequence to be expressed and separated, with one of the recombinase recognition sequences between the promoter and sequence to be expressed. In this embodiment the promoter and sequence to be expressed become operably linked upon site specific recombination.

In one embodiment the promoter is a light regulated promoter.

In one embodiment the promoter is the promoter of a chlorophyll a/b binding protein (cab) gene.

In one embodiment the promoter comprises a sequence with at least 70% identity to the sequence of SEQ ID NO:67.

In one embodiment the promoter comprises the sequence of SEQ ID NO:67.

Preferably the expression construct also comprises a terminator operably linked to the sequence to be expressed.

The sequence to be expressed may be the coding sequence encoding a polypeptide.

In one embodiment the polypeptide is an R2R3 MYB transcription factor, capable of regulating the production of anthocyanin in a plant.

In a further embodiment the polypeptide comprises a sequence with at least 70% identity to SEQ ID NO: 68 or 69.

In a further embodiment the polypeptide comprises a sequence with at least 70% identity to SEQ ID NO: 68.

In a further embodiment the polypeptide comprises a sequence with at least 70% identity to SEQ ID NO: 69.

In a further embodiment the polypeptide comprises the sequence of SEQ ID NO: 68.

In a further embodiment the polypeptide comprises the sequence of SEQ ID NO: 69.

Alternatively the sequence to be expressed may be a sequence suitable for effecting the silencing of at least one endogenous polynucleotide of polypeptide in a plant transformed with the expression construct.

Alternatively, between the recombinase recognition sequences, the vector comprises an intact plant gene.

Preferably the gene comprises a promoter, a coding sequence optionally including introns, and a terminator.

Alternatively the vector comprises, between the recombinase recognition sequences, at least one T-DNA border-like sequence, in place of the T-DNA border sequence.

### 4. Plant-Derived Minicircle (for Direct or Agrobacterium-Mediated Intragenic Transformation)

In a further aspect the invention provides a minicircle DNA molecule composed entirely of sequences derived from plant species.

In a preferred embodiment a minicircle DNA molecule is generated from a vector of the invention.

Preferably the minicircle DNA molecule is generated from a vector of the invention, by the action of a recombinase enzyme.

Preferably when the recombinase sites in the vector are loxP-like sequences, the recombinase is Cre.

Preferably when the recombinase sites in the vector are frt-like sequences, the recombinase is FLP.

Preferably the minicircle comprises at least one expression construct.

The expression construct preferably comprises a promoter, and a sequence to be expressed.

Preferably the promoter is operably linked to the sequence to be expressed.

In one embodiment the promoter is a light regulated promoter.

In one embodiment the promoter is the promoter of a chlorophyll a/b binding protein (cab) gene.

In one embodiment the promoter comprises a sequence with at least 70% identity to the sequence of SEQ ID NO:67.

In one embodiment the promoter comprises the sequence of SEQ ID NO:67.

Preferably the expression construct also comprises a terminator operably linked to the sequence to be expressed.

The sequence to be expressed may be the coding sequence encoding a polypeptide.

In one embodiment the polypeptide is an R2R3 MYB transcription factor, capable of regulating the production of anthocyanin in a plant.

In a further embodiment the polypeptide comprises a sequence with at least 70% identity to SEQ ID NO: 68 or 69.

In a further embodiment the polypeptide comprises a sequence with at least 70% identity to SEQ ID NO: 68.

In a further embodiment the polypeptide comprises a sequence with at least 70% identity to SEQ ID NO: 69.

In a further embodiment the polypeptide comprises the sequence of SEQ ID NO: 68.

In a further embodiment the polypeptide comprises the sequence of SEQ ID NO: 69.

Alternatively the sequence to be expressed may be a sequence suitable for effecting the silencing of at least one endogenous polynucleotide of polypeptide in a plant transformed with the expression construct.

The expression construct may also be an intact gene, such as a gene isolated from a plant. The intact gene may comprise a promoter, a coding sequence optionally including introns, and a terminator.

In a preferred embodiment the expression construct and the elements (promoter, sequence to be expressed, and terminator) within it are derived from plants. More preferably the expression construct and the elements within it are derived from a species interfertile with the plant species from which the recombinase recognition sequences, used to produce it, are derived. Most preferably, the expression construct and the elements within it, are derived from the same species as the plant species from which the recombinase recognition sequences, used to produce it, are derived.

The minicircle may also comprise a selectable marker sequence. Preferably the selectable marker sequence is derived from a plant species. More preferably the selectable marker sequence is derived from a species interfertile with the plant species from which the recombinase recognition sequences, used to produce the minicircle, are derived. Most preferably, the selectable marker sequence is derived from the same species as the plant species from which the recombinase recognition sequences, used to produce the minicircle, are derived.

### 5. Plant-Derived Minicircle (Useful for Agrobacterium-Mediated Intragenic Transformation)

In one embodiment, the minicircle molecule comprises at least one T-DNA border-like sequence.

In an alternative embodiment, the minicircle molecule comprises two T-DNA border-like sequences.

In a preferred embodiment, the minicircle molecule comprises one T-DNA border-like sequence.

Preferably the T-DNA border-like sequence or sequences is/are derived from a species interfertile with the plant species from which the recombinase recognition sequences, used to produce the minicircle, are derived. More preferably, the T-DNA border-like sequence or sequences is/are derived from the same species as the plant species from which the recombinase recognition sequences, used to produce the minicircle, are derived.

In a preferred embodiment, all of the sequence of the minicircle is derived from plant species, more preferably interfertile plant species, most preferably the same plant species.

### 6. Minicircles Useful for Agrobacterium-Mediated Transformation

In a further aspect the invention provides a minicircle DNA molecule comprising at least one T-DNA border sequence.

In an alternative embodiment, the minicircle molecule comprises two T-DNA border sequences.

In a preferred embodiment, the minicircle molecule comprises one T-DNA border sequence.

In a preferred embodiment a minicircle DNA molecule is generated from a vector of the invention.

Preferably the minicircle DNA molecule is generated from a vector of the invention, by the action of a recombinase enzyme.

Preferably the minicircle comprises at least one expression construct.

The expression construct preferably comprises a promoter, and a sequence to be expressed.

Preferably the promoter is operably linked to the sequence to be expressed.

In one embodiment the promoter is a light regulated promoter.

In one embodiment the promoter is the promoter of a chlorophyll a/b binding protein (cab) gene.

In one embodiment the promoter comprises a sequence with at least 70% identity to the sequence of SEQ ID NO:67.

In one embodiment the promoter comprises the sequence of SEQ ID NO:67.

Preferably the expression construct also comprises a terminator operably linked to the sequence to be expressed.

The sequence to be expressed may be the coding sequence encoding a polypeptide.

In one embodiment the polypeptide is an R2R3 MYB transcription factor, capable of regulating the production of anthocyanin in a plant.

In a further embodiment the polypeptide comprises a sequence with at least 70% identity to SEQ ID NO: 68 or 69.

In a further embodiment the polypeptide comprises a sequence with at least 70% identity to SEQ ID NO: 68.

In a further embodiment the polypeptide comprises a sequence with at least 70% identity to SEQ ID NO: 69.

In a further embodiment the polypeptide comprises the sequence of SEQ ID NO: 68.

In a further embodiment the polypeptide comprises the sequence of SEQ ID NO: 69.

Alternatively the sequence to be expressed may be a sequence suitable for effecting the silencing of at least one endogenous polynucleotide of polypeptide in a plant transformed with the expression construct.

Alternatively, the minicircle comprises an intact plant gene.

Preferably the gene comprises a promoter, a coding sequence, optionally including introns, and a terminator.

Alternatively the minicircle comprises, at least one T-DNA border-like sequence, in place of the T-DNA border sequence.

In a further aspect the invention provides a plant cell or plant transformed with a minicircle of the invention.

Once a plant is transformed with a minicircle DNA, the minicircle will have assumed a linear confirmation within the plant genome.

There for the phrase “plant cell or plant transformed with a minicircle” in intended to include a plant cell or plant transformed to include the linearised form of the minicircle in the plant or plant cells genome.

The invention also provides a plant tissue, organ, propagule or progeny of the plant cell or plant of the invention. The invention also provides a product, such as a food, feed or fibre products, produced from a plant, plant tissue, organ, propagule or progeny of the plant cell or plant of the invention. Preferably the plant, plant tissue, organ, propagule, progeny or product is transformed with a minicircle DNA molecule of the invention.

### 7. Method for Producing a Minicircle of the Invention

In a further aspect the invention provides a method for a minicircle, the method comprising contacting a vector of the invention with a recombinase, to produce a minicircle by site specific recombination.

Preferably when the recombinase sites in the vector are loxP or loxP-like sequences, the recombinase is Cre.

Preferably when the recombinase sites in the vector are frt or frt-like sequences, the recombinase is FLP.

Preferably the recombinase is expressed in a cell that comprises the vector.

Preferably the cell is a bacterial cell.

### 8. Transformation Method Using Plant-Derived or Non Plant Derived Minicircle DNA (Direct or Agrobacterium-Mediated Transformation)

In a further aspect the invention provides a method for transforming a plant, the method comprising introducing a minicircle DNA molecule into a plant cell, or plant to be transformed.

The minicircle DNA molecule may optionally be linearised prior to being introduced into the plant. The minicircle may be linearised by a restriction enzyme.

In a preferred embodiment, the minicircle is a minicircle of the invention.

In a further embodiment, the minicircle is produced from a vector of the invention by action of an appropriate recombinase.

In a preferred embodiment the minicircle DNA is composed entirely of sequence derived from plant species.

In a more preferred embodiment the minicircle DNA is composed entirely of sequence derived from plant species that are interfertile with the plant to be transformed.

In a yet more preferred embodiment the minicircle DNA is composed entirely of sequence derived from the same plant species as the plant to be transformed.

In one embodiment the minicircle DNA may comprise at least one expression construct as described above.

In a further embodiment the minicircle DNA may comprise at least one intact gene as described above.

In a further embodiment the minicircle DNA is incorporated into the genome of the plant.

In a further embodiment the method comprises the additional step of generating the minicircle DNA molecule from a vector, prior to introducing the minicircle into the plant.

Preferably the vector is a vector of the invention.

In a preferred embodiment the minicircle is generated by contacting a vector of the invention with a recombinase, to produce a minicircle by site specific recombination.

Preferably when the recombinase sites in the vector are loxP or loxP-like sequences, the recombinase is Cre.

Preferably when the recombinase sites in the vector are frt or frt-like sequences, the recombinase is FLP.

Preferably the recombinase is expressed in a cell that comprises the vector.

Preferably the cell is a bacterial cell.

In a preferred embodiment the transformed plant produced by the method is only transformed with plant-derived sequences.

More preferably the resulting transformed plant is only transformed with sequences that are derived from a plant species that is interfertile with the transformed plant.

Most preferably the resulting transformed plant is only transformed with sequences that are derived from the same species as the transformed plant.

In one embodiment transformation is vir gene-mediated.

In a further embodiment transformation is Agrobacterium-mediated.

When transformation is vir gene or Agrobacterium-mediated, the minicircle comprises at least one T-DNA border sequence or T-DNA border like sequence as described herein.

In an alternative embodiment transformation involves direct DNA uptake.

In a further aspect the invention provides a method for producing a plant cell or plant with a modified trait, the method comprising:
(a) transforming of a plant cell or plant with a minicircle DNA molecule comprising a genetic construct capable of altering expression of a gene which influences the trait; and
(b) obtaining a stably transformed plant cell or plant modified for the trait.

In one embodiment the minicircle is a minicircle of the invention.

In one embodiment transformation is vir gene-mediated.

In a further embodiment transformation is Agrobacterium-mediated.

When transformation is vir gene or Agrobacterium-mediated, the minicircle comprises at least one T-DNA border sequence or T-DNA border like sequence as described herein.

In an alternative embodiment transformation involves direct DNA uptake.

The invention provides a plant cell or plant produced by a method of the invention.

The invention also provides a plant tissue, organ, propagule or progeny of the plant cell or plant of the invention. The invention also provides a product, such as a food, feed or fibre products, produced from a plant, plant tissue, organ, propagule or progeny of the plant cell or plant of the invention. Preferably the plant, plant tissue, organ, propagule, progeny or product is transformed with a minicircle DNA molecule of the invention.

### DETAILED DESCRIPTION

### Definitions

### Recombinase Recognition Sequences and Recombinases

Previously site-specific recombination systems have been elegantly used to excise precise sequences such as selectable marker constructs in transgenic plants (reviewed by Gilbertson, L. Cre-lox recombination: Cre-ative tools for plant biotechnology TRENDS in Biotechnology 21(12) 550-555 2003).

Two such recombination systems are the Escherichia coli bacteriophage P1 Cre/loxP system and the Saccharomyces cerevisiae FLP/frt systems, which require only a single-polypeptide recombinase, Cre or FLP and minimal 34 bp DNA recombination sites, loxP or frt.

When two recombination sites in the same orientation flank DNA sequence, recombinase mediates a crossover between these sites effectively excising the intervening DNA.

Following excision only one recombination site remains.

The term “recombinase recognition sequence” means a sequence that is recognised by a recombinase to result in the site specific recombination described above.

Of the many types of recombinase recognition sequences known, two types are particularly well studied. The first are loxP sequences, which are recombined by the action of the Cre recombinase enzyme (Hoess, R. H., and K. Abremski. 1985. Mechanism of strand cleavage and exchange in the Cre-lox site-specific recombination system. J. Mol. Biol. 181:351-362.). The second is frt sequences, which are recombined by action of an FLP recombinase enzyme (Sadowski, P. D. 1995. The Flp recombinase of the 2-microns plasmid of Saccharomyces cerevisiae. Prog. Nucleic Acid Res. Mol. Biol. 51:53-91.).

A loxP sequence is typically between 24-100 bp in length, preferably 24-80 bp in length, preferably 24-70 bp in length, preferably 24-60 bp in length, preferably 24-50 bp in length, preferably 24-40 bp in length, preferably 24-34 bp in length, preferably 26-34 bp in length, preferably 28-34 bp in length, preferably 30-34 bp in length, preferably 32-34 bp in length, preferably 34 bp in length.

A loxP sequence preferably comprises the consensus motif

(SEQ ID NO: 64)
5′ ATAACTTCGTATANNNNNNNNTATACGAAGTTAT 3′

(where N=any nucleotide).

The term “loxP-like sequence” refers to a sequence derived from the genome of a plant which can perform the function of a Cre recombinase recognition site. The loxP-like sequence may be comprised of one contiguous sequence found in the genome of a plant or may be formed by combining two or more fragments found in the genome of a plant.

A loxP-like sequence is, between 24-100 bp in length, preferably 24-80 bp in length, preferably 24-70 bp in length, preferably 24-60 bp in length, preferably 24-50 bp in length, preferably 24-40 bp in length, preferably 24-34 bp in length, preferably 26-34 bp in length, preferably 28-34 bp in length, preferably 30-34 bp in length, preferably 32-34 bp in length, preferably 34 bp in length.

A loxP-like sequence preferably comprises the consensus motif

(SEQ ID NO: 64)
5′ ATAACTTCGTATANNNNNNNNTATACGAAGTTAT 3′

(where N=any nucleotide).

Preferably the loxP-like sequence is not identical to any loxP sequence present in a non-plant species.

loxP-like sequences from multiple plant species and methods for identifying and producing them are described in WO05/121346 (which is incorporated herein by reference in its entirety) and in Example 5.

An sequence is typically between 28-100 bp in length, preferably 28-80 bp in length, preferably 28-70 bp in length, preferably 28-60 bp in length, preferably 28-50 bp in length, preferably 28-40 bp in length, preferably 28-34 bp in length, preferably 30-34 bp in length, preferably 32-34 bp in length, preferably 34 bp in length.

A frt sequence preferably comprises the consensus motif

(SEQ ID NO: 65)
5′ GAAGTTCCTATACNNNNNNNNGWATAGGAACTTC 3′

(where W=A or T, N=any nucleotide).

The consensus motif may include an additional nucleotide at the 5′ end. Preferably the additional nucleotide is an A or a T.

The term “frt-like sequence” refers to a sequence derived from the genome of a plant which can perform the function of an FLP recombinase recognition site. The frt-like sequence may be comprised of one contiguous sequence found in the genome of a plant or may be formed by combining two sequence fragments found in the genome of a plant.

An frt-like sequence is between 28-100 bp in length, preferably 28-80 bp in length, preferably 28-70 bp in length, preferably 28-60 bp in length, preferably 28-50 bp in length, preferably 28-40 bp in length, preferably 28-34 bp in length, preferably 30-34 bp in length, preferably 32-34 bp in length, preferably 34 bp in length.

A frt-like sequence preferably comprises the consensus motif

(SEQ ID NO: 65)
5′ GAAGTTCCTATACNNNNNNNNGWATAGGAACTTC 3′

(where W=A or T, N=any nucleotide).

The consensus motif may include an additional nucleotide at the 5′ end. Preferably the additional nucleotide is an A or a T.

Preferably the frt-like sequence is not identical to any frt sequence present in a non-plant species.

frt-like sequences from multiple plant species and methods for identifying and producing them are described in WO05/121346 (which is incorporated herein by reference in its entirety) and in Example 6.

T-DNA border sequences are well known to those skilled in the art and are described for example in Wang et al (Molecular and General Genetics, Volume 210, Number 2, December, 1987), as well as numerous other well-known references.

The term “T-DNA border-like sequence” refers to a sequence derived from the genome of a plant which can perform the function of an Agrobacterium T-DNA border sequence in integration of a polynucleotide sequence into the genome of a plant. The T-DNA border-like sequence may be comprised of one contiguous sequence found in the genome of a plant or may be formed by combining two or more sequences found in the genome of a plant.

A T-DNA border-like sequence is between 10-100 bp in length, preferably 10-80 bp in length, preferably 10-70 bp in length, preferably 15-60 bp in length, preferably 15-50 bp in length, preferably 15-40 bp in length, preferably 15-30 bp in length, preferably 20-30 bp in length, preferably 21-30 bp in length, preferably 22-30 bp in length, preferably 23-30 bp in length, preferably 24-30 bp in length, preferably 25-30 bp in length, preferably 26-30 bp in length.

A T-DNA border-like sequence preferably comprises the consensus motif:

| | |
|---|---|
| 5′GRCAGGATATATNNNNNKSTMAWN3′ | (SEQ ID NO: 66) |

(where R=G or A, K=T or G, S=G or C, M=C or A, W=A or T and N=any nucleotide).

The T-DNA border-like sequence of the invention is preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 95%, more preferably at least 99% identical to any Agrobacterium T-DNA border sequence. Preferably the T-DNA border-like sequence is less than 100% identical to any Agrobacterium T-DNA border sequence.

Although not preferred, a T-DNA border-like sequence of the invention may include a sequence naturally occurring in a plant which is modified or mutated to change the efficiency at which it is capable of integrating a linked polynucleotide sequence into the genome of a plant.

T-DNA border-like sequences from multiple plant species and methods for identifying and producing them are described in WO05/121346, which is incorporated herein by reference in its entirety.

The term “plant-derived sequence”, means sequence that is the same as sequence present in a plant. A “plant-derived sequence” may be composed of one or more contigous sequence fragments that are present at separate locations in the genome of a plant. Preferably at least one of the sequence fragments is at least 5 nucleotides in length, more preferably at least 6, more preferably at least 7, more preferably at least 8, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20, more preferably at least 21, more preferably at least 22, more preferably at least 23, more preferably at least 24, more preferably at least 25 nucleotide in length.

A “plant-derived sequence” may be produce synthetically or recombinantly, provided it meets the definition above.

The term “minicircle” means a DNA molecule typically devoid of any of plasmid/vector backbone sequences. Minicircles can be generated in vivo from bacterial plasmids by site-specific intramolecular recombination between recombinase recognition sites in the plasmid, to result in a minicircle DNA vectors devoid of bacterial plasmid backbone DNA [Darquet et al 1997, 1999].

The terms “minicircle” and minicircle DNA molecule can be used interchangeably throughout this specification.

The term “between the recombinase recognition sequences” means within the region of a vector comprising the recombinase recognition sequences that will form the minicircle when the vector is contacted with the appropriate recombinase. That is, sequences between the recombinase recognition sequences will form part of the minicircle produced by the action of the appropriate recombinase.

The term “outside the recombinase recognition sequences” means within the region of a vector comprising the recombinase recognition sequences that will not form the minicircle when the vector is contacted with the appropriate recombinase. Sequences outside the recombinase recognition sequences may optionally include non-plant sequences such as origins of replication for bacteria, or selectable markers for bacteria. Sequences “outside the recombinase recognition sequences” will also form a circular DNA molecule, but this molecule is distinct from the minicircle.

The terms “selectable marker derived from a plant” or “plant-derived selectable marker” or grammatical equivalents thereof refers to a sequence derived from a plant which can enable selection of a plant cell harbouring the sequence or a sequence to which the selectable marker is linked. The “plant-derived selectable markers” may be composed of one, two or more sequence fragments derived from plants. Preferably the “plant-derived selectable markers” are composed of two sequence fragments derived from plants.

Plant-derived selectable marker sequences which are useful for selecting transformed plant cells and plants harbouring a particular sequence include PPga22 (Zuo et al., Curr Opin Biotechnol. 13: 173-80, 2002), Ckil (Kakimoto, Science 274: 982-985, 1996), Esrl (Banno et al., Plant Cell 13: 2609-18, 2001), and dhdps-r1 (Ghislain et al., Plant Journal, 8: 733-743, 1995). It is also possible to use pigmentation markers to visually select transformed plant cells and plants, such as the R and Cl genes (Lloyd et al., Science, 258: 1773-1775, 1992; Bodeau and Walbot, Molecular and General Genetics, 233: 379-387, 1992).

“Plant-derived selectable markers” from multiple plant species and methods for identifying and producing them are also described in WO05/121346, which is incorporated herein by reference in its entirety.

The term “MYB transcription factor” is a term well understood by those skilled in the art to refer to a class of transcription factors characterised by a structurally conserved DNA binding domain consisting of single or multiple imperfect repeats.

The term “R2R3 MYB transcription factor” is a term well understood by those skilled in the art to refer to MYB transcription factors of the two-repeat class.

The term “light-regulated promoter” is a term well understood by those skilled in the art to mean a promoter that controls expression of an operably linked sequence in a ight regulated manner. Light regulated promoters are well-known to those skilled in the art (Annual Review of Plant Physiology and Plant Molecular Biology. 1998, Vol. 49: 525-555). Examples of light-regulated promoters include cholophyll a/b binding protein (cab) gene promoters, and small subunit of rubisco (rbcs) promoters.

The term “polynucleotide(s),” as used herein, means a single or double-stranded deoxyribonucleotide or ribonucleotide polymer of any length, and include as non-limiting examples, coding and non-coding sequences of a gene, sense and antisense sequences, exons, introns, genomic DNA, cDNA, pre-mRNA, mRNA, rRNA, siRNA, miRNA, tRNA, ribozymes, recombinant polynucleotides, isolated and purified naturally occurring DNA or RNA sequences, synthetic RNA and DNA sequences, nucleic acid probes, primers, fragments, genetic constructs, vectors and modified polynucleotides.

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. In certain embodiments, variants of the inventive polypeptides and polynucleotides possess biological activities that are the same or similar to those of the inventive polypeptides or polynucleotides. The term “variant” with reference to polynucleotides and polypeptides encompasses all forms of polynucleotides and polypeptides as defined herein.

Variant polynucleotide sequences preferably exhibit at least 50%, more preferably at least 70%, more preferably at least 80%, more preferably at least 90%, more preferably at least 95%, more preferably at least 98%, and most preferably at least 99% identity to a sequence of the present invention. Identity is found over a comparison window of at least 5 nucleotide positions; preferably at least 10 nucleotide positions, preferably at least 20 nucleotide positions, preferably at least 50 nucleotide positions, more preferably at least 100 nucleotide positions, and most preferably over the entire length of a polynucleotide of the invention.

Polynucleotide sequence identity can be determined in the following manner. The subject polynucleotide sequence is compared to a candidate polynucleotide sequence using BLASTN (from the BLAST suite of programs, version 2.2.5 [Nov. 2002]) in bl2seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI (ftp://ftp.ncbi.nih.gov/blast/). The default parameters of bl2seq may be utilized.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program may be used which computes an optimal global, alignment of two sequences without penalizing terminal gaps. GAP is described in the following paper: Huang, X. (1994) On Global Sequence Alignment. Computer Applications in the Biosciences 10, 227-235.

Alternatively, variant polynucleotides of the present invention hybridize to the polynucleotide sequences disclosed herein, or complements thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or northern blot) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing,). Tm for polynucleotide molecules greater than about 100 bases can be calculated by the formula Tm=81. 5+0.41% (G+C-log(Na+) (Sambrook et al., Eds, 1987, Molecular Cloning, A Laboratory Manual, 2nd Ed. Cold Spring Harbor Press; Bolton and McCarthy, 1962, PNAS 84:1390). Typical stringent conditions for polynucleotide molecules of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

With respect to polynucleotide molecules having a length less than 100 bases, exemplary stringent hybridization conditions are 5 to 10° C. below Tm. On average, the Tm of a polynucleotide molecule of length less than 100 bp is reduced by approximately (500/oligonucleotide length)° C.

Variant polynucleotides of the present invention also encompasses polynucleotides that differ from the sequences of the invention but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

Variant polynucleotides due to silent variations and conservative substitutions in the encoded polypeptide sequence may be determined using the publicly available bl2seq program from the BLAST suite of programs (version 2.2.5 [Nov. 2002]) from NCBI (ftp://ftp.ncbi.nih.gov/blast/) via the tblastx algorithm as previously described.

A “fragment” of a polynucleotide sequence provided herein is a subsequence of contiguous nucleotides that is at least 5 nucleotides in length. The fragments of the invention comprise at least 5 nucleotides, preferably at least 10 nucleotides, preferably at least 15 nucleotides, preferably at least 20 nucleotides, more preferably at least 30 nucleotides, more preferably at least 50 nucleotides, more preferably at least 50 nucleotides and most preferably at least 60 nucleotides of contiguous nucleotides of a specified polynucleotide or section of a plant genome.

The term “primer” refers to a short polynucleotide, usually having a free 3′OH group, that is hybridized to a template and used for priming polymerization of a polynucleotide complementary to the target.

The term “probe” refers to a short polynucleotide that is used to detect a polynucleotide sequence that is complementary to the probe, in a hybridization-based assay. The probe may consist of a “fragment” of a polynucleotide as defined herein.

The term “polypeptide”, as used herein, encompasses amino acid chains of any length, including full-length proteins, in which amino acid residues are linked by covalent peptide bonds. Polypeptides of the present invention may be purified natural products, or may be produced partially or wholly using recombinant or synthetic techniques. The term may refer to a polypeptide, an aggregate of a polypeptide such as a dimer or other multimer, a fusion polypeptide, a polypeptide fragment, a polypeptide variant, or derivative thereof.

The term “isolated” as applied to the polynucleotide sequences disclosed herein is used to refer to sequences that are removed from their natural cellular environment. An isolated molecule may be obtained by any method or combination of methods including biochemical, recombinant, and synthetic techniques.

The term “genetic construct” refers to a polynucleotide molecule, usually double-stranded DNA, which may have inserted into it another polynucleotide molecule (the insert polynucleotide molecule) such as, but not limited to, a cDNA molecule. A genetic construct may contain the necessary elements that permit transcribing the insert polynucleotide molecule, and, optionally, translating the transcript into a polypeptide. The insert polynucleotide molecule may be derived from the host cell, or may be derived from a different cell or organism and/or may be a recombinant or synthetic polynucleotide. Once inside the host cell the genetic construct may become integrated in the host chromosomal DNA. The term “genetic construct” includes “expression construct” as herein defined. The genetic construct may be linked to a vector.

The term “expression construct” refers to a genetic construct that includes the necessary elements that permit transcribing the insert polynucleotide molecule, and, optionally, translating the transcript into a polypeptide. An expression construct typically comprises in a 5′ to 3′ direction:
a) a promoter functional in the host cell into which the construct will be transformed,
b) the polynucleotide to be transcribed and/or expressed, and optionally
c) a terminator functional in the host cell into which the construct will be transformed.

In one embodiment the order of these three components of an expression construct can be altered when assembled on a vector between the recombination recognition sequences. The correct order is then reassembled by intramolecular site-specific recombination upon formation of the minicircle for plant transformation. This may involve the positioning of a promoter just inside one recombination recognition sequence and the remainder of the expression construct just inside the second recombination recognition sequence. Alternatively the expression construct could be split elsewhere, such as within an intron region. Induction of the recombinase activity then mediates a crossover event between the recombination recognition sequences to restore the components of the expression construct in the desired 5′ to 3′ direction. In this manner an expression construct will be non-functional as assembled on the vector, but becomes functional upon formation of the minicircle. In another embodiment, the assembly of marker gene for plant transformation in this manner provides a method to preferentially select transformed plant cells and plants derived from minicircles, especially for Agrobacterium-mediated transformation. This approach is used in Example 3, part B and Example 4, part A.

The term “vector” refers to a polynucleotide molecule, usually double stranded DNA, which may include a genetic construct. The vector may be capable of replication in at least one host system, such as Escherichia coli.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence is identified by the presence of a 5′ translation start codon and a 3′ translation stop codon. When inserted into a genetic construct, a “coding sequence” is capable of being expressed when it is operably linked to promoter and terminator sequences.

“Operably-linked” means that the sequence to be expressed is placed under the control of regulatory elements that include promoters, tissue-specific regulatory elements, temporal regulatory elements, chemical-inducible regulatory elements, environment-inducible regulatory elements, enhancers, repressors and terminators.

The term “noncoding region” refers to untranslated sequences that are upstream of the translational start site and downstream of the translational stop site. These sequences are also referred to respectively as the 5′ UTR and the 3′ UTR. These regions include elements required for transcription initiation and termination and for regulation of translation efficiency.

Terminators are sequences, which terminate transcription, and are found in the 3′ untranslated ends of genes downstream of the translated sequence. Terminators are important determinants of mRNA stability and in some cases have been found to have spatial regulatory functions.

The term “promoter” refers to nontranscribed cis-regulatory elements upstream of the coding region that regulate gene transcription. Promoters comprise cis-initiator elements which specify the transcription initiation site and conserved boxes such as the TATA box, and motifs that are bound by transcription factors.

A “transformed plant” refers to a plant which contains new genetic material as a result of genetic manipulation or transformation. The new genetic material may be derived from a plant of the same species, an interfertile species, or a different species from the plant transformed.

An “inverted repeat” is a sequence that is repeated, where the second half of the repeat is in the complementary strand, e.g.,

(5′)GATCTA . . . TAGATC(3′)
(3′)CTAGAT . . . ATCTAG(5′)

Read-through transcription will produce a transcript that undergoes complementary base-pairing to form a hairpin structure provided that there is a 3-5 bp spacer between the repeated regions.

The terms “to alter expression of” and “altered expression” of a polynucleotide or polypeptide, are intended to encompass the situation where genomic DNA corresponding to a polynucleotide is modified thus leading to altered expression of a corresponding polynucleotide or polypeptide. Modification of the genomic DNA may be through genetic transformation or other methods known in the art for inducing mutations. The “altered expression” can be related to an increase or decrease in the amount of messenger RNA and/or polypeptide produced and may also result in altered activity of a polypeptide due to alterations in the sequence of a polynucleotide and polypeptide produced.

Methods for transforming plant cells, plants and portions thereof with polynucleotides are described in Draper et al., 1988, Plant Genetic Transformation and Gene Expression: A Laboratory Manual. Blackwell Sci. Pub. Oxford, p. 365; Potrykus and Spangenburg, 1995, Gene Transfer to Plants. Springer-Verlag, Berlin; and Gelvin et al., 1993, Plant Molecular Biol. Manual. Kluwer Acad. Pub. Dordrecht. A review of transgenic plants, including transformation techniques, is provided in Galun and Breiman, 1997, Transgenic Plants. Imperial College Press, London.

It will be well understood by those skilled in the art that the minicircle DNA molecules of the invention can function in the place of the co-intergrate or binary vectors for Agrobacterium-mediated transformation and as vectors for direct DNA uptake approaches.

The polynucleotide molecules of the invention can be isolated by using a variety of techniques known to those of ordinary skill in the art. By way of example, such polynucleotides can be isolated through use of the polymerase chain reaction (PCR) described in Mullis et al., Eds. 1994 The Polymerase Chain Reaction, Birkhauser, incorporated herein by reference. The polynucleotides of the invention can be amplified using primers, as defined herein, derived from the polynucleotide sequences of the invention.

Further methods for isolating polynucleotides of the invention include use of all, or portions of, the disclosed polynucleotide sequences as hybridization probes. The technique of hybridizing labeled polynucleotide probes to polynucleotides immobilized on solid supports such as nitrocellulose filters or nylon membranes, can be used to screen the genomic or cDNA libraries. Exemplary hybridization and wash conditions are: hybridization for 20 hours at 65° C. in 5.0×SSC, 0.5% sodium dodecyl sulfate, 1×Denhardt's solution; washing (three washes of twenty minutes each at 55° C.) in 1.0×SSC, 1% (w/v) sodium dodecyl sulfate, and optionally one wash (for twenty minutes) in 0.5×SSC, 1% (w/v) sodium dodecyl sulfate, at 60° C. An optional further wash (for twenty minutes) can be conducted under conditions of 0.1×SSC, 1% (w/v) sodium dodecyl sulfate, at 60° C.

The polynucleotide fragments of the invention may be produced by techniques well-known in the art such as restriction endonuclease digestion and oligonucleotide synthesis.

A partial polynucleotide sequence may be used, in methods well-known in the art to identify the corresponding further contiguous polynucleotide sequence. Such methods would include PCR-based methods, 5′RACE (Frohman M A, 1993, Methods Enzymol. 218: 340-56) and hybridization-based method, computer/database-based methods. Further, by way of example, inverse PCR permits acquisition of unknown sequences, flanking the polynucleotide sequences disclosed herein, starting with primers based on a known region (Triglia et al., 1998, Nucleic Acids Res 16, 8186, incorporated herein by reference). The method uses several restriction enzymes to generate a suitable fragment in the known region of a gene. The fragment is then circularized by intramolecular ligation and used as a PCR template. Divergent primers are designed from the known region. In order to physically assemble full-length clones, standard molecular biology approaches can be utilized (Sambrook et al., Molecular Cloning: A Laboratory Manual, 2nd Ed. Cold Spring Harbor Press, 1987).

It will be understood by those skilled in the art that in order to produce intragenic vectors for further species it may be necessary to identify the sequences corresponding to essential or preferred elements of such vectors in other plant species. It will be appreciated by those skilled in the art that this may be achieved by identifying polynucleotide variants of the sequences disclosed. Many methods are known by those skilled in the art for isolating such variant sequences.

Variant polynucleotides may be identified using PCR-based methods (Mullis et al., Eds. 1994 The Polymerase Chain Reaction, Birkhauser). Typically, the polynucleotide sequence of a primer, useful to amplify variants of polynucleotide molecules of the invention by PCR, may be based on a sequence encoding a conserved region of the corresponding amino acid sequence.

Further methods for identifying variant polynucleotides of the invention include use of all, or portions of, the polynucleotides disclosed herein as hybridization probes to screen plant genomic or cDNA libraries as described above. Typically probes based on a sequence encoding a conserved region of the corresponding amino acid sequence may be used. Hybridisation conditions may also be less stringent than those used when screening for sequences identical to the probe.

The variant polynucleotide sequences of the invention may also be identified by computer-based methods well-known to those skilled in the art, using public domain sequence alignment algorithms and sequence similarity search tools to search sequence databases (public domain databases include Genbank, EMBL, Swiss-Prot, PIR and others). See, e.g., Nucleic Acids Res. 29: 1-10 and 11-16, 2001 for examples of online resources. Similarity searches retrieve and align target sequences for comparison with a sequence to be analyzed (i.e., a query sequence). Sequence comparison algorithms use scoring matrices to assign an overall score to each of the alignments.

An exemplary family of programs useful for identifying variants in sequence databases is the BLAST suite of programs (version 2.2.5 [Nov. 2002]) including BLASTN, BLASTP, BLASTX, tBLASTN and tBLASTX, which are publicly available from (ftp://ftp.ncbi.nih.gov/blast/) or from the National Center for Biotechnology Information (NCBI), National Library of Medicine, Building 38A, Room 8N805, Bethesda, Md. 20894 USA. The NCBI server also provides the facility to use the programs to screen a number of publicly available sequence databases. BLASTN compares a nucleotide query sequence against a nucleotide sequence database. BLASTP compares an amino acid query sequence against a protein sequence database. BLASTX compares a nucleotide query sequence translated in all reading frames against a protein sequence database. tBLASTN compares a protein query sequence against a nucleotide sequence database dynamically translated in all reading frames. tBLASTX compares the six-frame translations of a nucleotide query sequence against the six-frame translations of a nucleotide sequence database. The BLAST programs may be used with default parameters or the parameters may be altered as required to refine the screen.

The use of the BLAST family of algorithms, including BLASTN, BLASTP, and BLASTX, is described in the publication of Altschul et al., Nucleic Acids Res. 25: 3389-3402, 1997.

The “hits” to one or more database sequences by a queried sequence produced by BLASTN, BLASTP, BLASTX, tBLASTN, tBLASTX, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN, BLASTP, BLASTX, tBLASTN and tBLASTX algorithms also produce “Expect” values for alignments. The Expect value (E) indicates the number of hits one can “expect” to see by chance when searching a database of the same size containing random contiguous sequences. The Expect value is used as a significance threshold for determining whether the hit to a database indicates true similarity. For example, an E value of 0.1 assigned to a polynucleotide hit is interpreted as meaning that in a database of the size of the database screened, one might expect to see 0.1 matches over the aligned portion of the sequence with a similar score simply by chance. For sequences having an E value of 0.01 or less over aligned and matched portions, the probability of finding a match by chance in that database is 1% or less using the BLASTN, BLASTP, BLASTX, tBLASTN or tBLASTX algorithm.

To identify the polynucleotide variants most likely to be functional equivalents of the disclosed sequences, several further computer based approaches are known to those skilled in the art.

Multiple sequence alignments of a group of related sequences can be carried out with CLUSTALW (Thompson, J. D., Higgins, D. G. and Gibson, T. J. (1994) CLUSTALW: improving the sensitivity of progressive multiple sequence alignment through sequence weighting, positions-specific gap penalties and weight matrix choice. Nucleic Acids Research, 22:4673-4680, http://www-igbmc.u-strasbg.fr/BioInfo/ClustalW/Top.html) or T-COFFEE (Cedric Notredame, Desmond G. Higgins, Jaap Hering a, T-Coffee: A novel method for fast and accurate multiple sequence alignment, J. Mol. Biol. (2000) 302: 205-217)) or PILEUP, which uses progressive, pairwise alignments (Feng and Doolittle, 1987, J. Mol. Evol. 25, 351).

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch and Bucher, 1994, Nucleic Acids Res. 22, 3583; Hofmann et al., 1999, Nucleic Acids Res. 27, 215) is a method of identifying the functions of uncharacterized proteins translated from genomic or cDNA sequences. The PROSITE database (www.expasy.org/prosite) contains biologically significant patterns and profiles and is designed so that it can be used with appropriate computational tools to assign a new sequence to a known family of proteins or to determine which known domain(s) are present in the sequence (Falquet et al., 2002, Nucleic Acids Res. 30, 235). Prosearch is a tool that can search SWISS-PROT and EMBL databases with a given sequence pattern or signature.

The function of a variant of a polynucleotide of the invention may be assessed by replacing the corresponding sequence in a vector or minicircle with the variant sequence and testing the functionality of the vector or minicircle in a host bacterial cell or in a plant transformation procedure as herein defined.

Methods for assembling and manipulating genetic constructs and vectors are well known in the art and are described generally in Sambrook et al., Molecular Cloning: A Laboratory Manual, 2nd Ed. Cold Spring Harbor Press, 1987; Ausubel et al., Current Protocols in Molecular Biology, Greene Publishing, 1987).

Numerous traits in plants may also be altered through methods of the invention. Such methods may involve the transformation of plant cells and plants, using a vector of the invention including a genetic construct designed to alter expression of a polynucleotide or polypeptide which modulates such a trait in plant cells and plants. Such methods also include the transformation of plant cells and plants with a combination of the construct of the invention and one or more other constructs designed to alter expression of one or more polynucleotides or polypeptides which modulate such traits in such plant cells and plants.

A number of plant transformation strategies are available (e.g. Birch, 1997, Ann Rev Plant Phys Plant Mol Biol, 48, 297). For example, strategies may be designed to increase expression of a polynucleotide/polypeptide in a plant cell, organ and/or at a particular developmental stage where/when it is normally expressed or to ectopically express a polynucleotide/polypeptide in a cell, tissue, organ and/or at a particular developmental stage which/when it is not normally expressed. The expressed polynucleotide/polypeptide may be derived from the plant species to be transformed or may be derived from a different plant species.

Transformation strategies may be designed to reduce expression of a polynucleotide/polypeptide in a plant cell, tissue, organ or at a particular developmental stage which/when it is normally expressed. Such strategies are known as gene silencing strategies.

Direct gene transfer involves the uptake of naked DNA by cells and its subsequent integration into the genome (Conner, A. J. and Meredith, C. P., Genetic manipulation of plant cells, pp. 653-688, in The Biochemistry of Plants: A Comprehensive Treatise, Vol 15, Molecular Biology, editor Marcus, A., Academic Press, San Diego, 1989; Petolino, J. Direct DNA delivery into intact cells and tissues, pp. 137-143, in Transgenic Plants and Crops, editors Khachatourians et al., Marcel Dekker, New York, 2002. The cells can include those of intact plants, pollen, seeds, intact plant organs, in vitro cultures of plants, plant parts, tissues and cells or isolated protoplasts. Those skilled in the art will understand that methods to effect direct DNA transfer may involve, but not limited to: passive uptake; the use of electroporation; treatments with polyethylene glycol and related chemicals and their adjuncts; electrophoresis, cell fusion with liposomes or spheroplasts; microinjection, silicon carbide whiskers, and microparticle bombardment.

Genetic constructs for expression of genes in transgenic plants typically include promoters for driving the expression of one or more cloned polynucleotide, terminators and selectable marker sequences to detect presence of the genetic construct in the transformed plant.

The promoters suitable for use in the constructs of this invention are functional in a cell, tissue or organ of a monocot or dicot plant and include cell-, tissue- and organ-specific promoters, cell cycle specific promoters, temporal promoters, inducible promoters, constitutive promoters that are active in most plant tissues, and recombinant promoters. Choice of promoter will depend upon the temporal and spatial expression of the cloned polynucleotide, so desired. The promoters may be those normally associated with a transgene of interest, or promoters which are derived from genes of other plants, viruses, and plant pathogenic bacteria and fungi. Those skilled in the art will, without undue experimentation, be able to select promoters that are suitable for use in modifying and modulating plant traits using genetic constructs comprising the polynucleotide sequences of the invention. Examples of constitutive promoters used in plants include the CaMV 35S promoter, the nopaline synthase promoter and the octopine synthase promoter, and the Ubi 1 promoter from maize. Plant promoters which are active in specific tissues, respond to internal developmental signals or external abiotic or biotic stresses are also described in the scientific literature. Exemplary promoters are described, e.g., in WO 02/00894, which is herein incorporated by reference.

Exemplary terminators that are commonly used in plant transformation genetic constructs include, e.g., the cauliflower mosaic virus (CaMV) 35S terminator, the Agrobacterium tumefaciens nopaline synthase or octopine synthase terminators, the Zea mays zein gene terminator, the Oryza sativa ADP-glucose pyrophosphorylase terminator and the Solanum tuberosum PI-II terminator.

Selectable markers commonly used in plant transformation include the neomycin phophotransferase II gene (NPT II) which confers kanamycin resistance, the aadA gene, which confers spectinomycin and streptomycin resistance, the phosphinothricin acetyl transferase (bar gene) for Ignite (AgrEvo) and Basta (Hoechst) resistance, and the hygromycin phosphotransferase gene (hpt) for hygromycin resistance.

It will be understood by those skilled in the art that non-plant derived regulatory elements described above may be used in the intragenic vectors of the invention operably linked to selectable markers placed between the recombinase recognition sites.

Gene silencing strategies may be focused on the gene itself or regulatory elements which effect expression of the encoded polypeptide. “Regulatory elements” is used here in the widest possible sense and includes other genes which interact with the gene of interest.

Genetic constructs designed to decrease or silence the expression of a polynucleotide/polypeptide of the invention may include an antisense copy of a polynucleotide of the invention. In such constructs the polynucleotide is placed in an antisense orientation with respect to the promoter and terminator.

An “antisense” polynucleotide is obtained by inverting a polynucleotide or a segment of the polynucleotide so that the transcript produced will be complementary to the mRNA transcript of the gene, e.g.,

5′GATCTA 3′ (coding strand)
3′CTAGAT 5′ (antisense strand)
3′CUAGAU 5′ mRNA
5′GAUCUA 3′ antisense RNA

Genetic constructs designed for gene silencing may also include an inverted repeat as herein defined. The preferred approach to achieve this is via RNA-interference strategies using genetic constructs encoding self-complementary “hairpin” RNA (Wesley et al., 2001, Plant Journal, 27: 581-590).

The transcript formed may undergo complementary base pairing to form a hairpin structure. Usually a spacer of at least 3-5 bp between the repeated region is required to allow hairpin formation.

Another silencing approach involves the use of a small antisense RNA targeted to the transcript equivalent to an miRNA (Llave et al., 2002, Science 297, 2053). Use of such small antisense RNA corresponding to polynucleotide of the invention is expressly contemplated.

The term genetic construct as used herein also includes small antisense RNAs and other such polynucleotides effecting gene silencing.

Transformation with an expression construct, as herein defined, may also result in gene silencing through a process known as sense suppression (e.g. Napoli et al., 1990, Plant Cell 2, 279; de Carvalho Niebel et al., 1995, Plant Cell, 7, 347). In some cases sense suppression may involve over-expression of the whole or a partial coding sequence but may also involve expression of non-coding region of the gene, such as an intron or a 5′ or 3′ untranslated region (UTR). Chimeric partial sense constructs can be used to coordinately silence multiple genes (Abbott et al., 2002, Plant Physiol. 128(3): 844-53; Jones et al., 1998, Planta 204: 499-505). The use of such sense suppression strategies to silence the expression of a polynucleotide of the invention is also contemplated.

The polynucleotide inserts in genetic constructs designed for gene silencing may correspond to coding sequence and/or non-coding sequence, such as promoter and/or intron and/or 5′ or 3′ UTR sequence, or the corresponding gene.

Other gene silencing strategies include dominant negative approaches and the use of ribozyme constructs (McIntyre, 1996, Transgenic Res, 5, 257).

Pre-transcriptional silencing may be brought about through mutation of the gene itself or its regulatory elements. Such mutations may include point mutations, frameshifts, insertions, deletions and substitutions.

The following are representative publications disclosing genetic transformation protocols that can be used to genetically transform the following plant species: onions (WO00/44919); peas (Grant et al., 1995 Plant Cell Rep., 15, 254-258; Grant et al., 1998, Plant Science, 139:159-164); petunia (Deroles and Gardner, 1988, Plant Molecular Biology, 11: 355-364); Medicago truncatula (Trieu and Harrison 1996, Plant Cell Rep. 16: 6-11); rice (Alam et al., 1999, Plant Cell Rep. 18, 572); maize (U.S. Pat. Nos. 5,177,010 and 5,981,840); wheat (Ortiz et al., 1996, Plant Cell Rep. 15, 1996, 877); tomato (U.S. Pat. No. 5,159,135); potato (Kumar et al., 1996 Plant J. 9, 821); cassaya (Li et al., 1996 Nat. Biotechnology 14, 736); lettuce (Michelmore et al., 1987, Plant Cell Rep. 6, 439); tobacco (Horsch et al., 1985, Science 227, 1229); cotton (U.S. Pat. Nos. 5,846,797 and 5,004,863); grasses (U.S. Pat. Nos. 5,187,073 and 6,020,539); peppermint (Niu et al., 1998, Plant Cell Rep. 17, 165); citrus plants (Pena et al., 1995, Plant Sci. 104, 183); caraway (Krens et al., 1997, Plant Cell Rep, 17, 39); banana (U.S. Pat. No. 5,792,935); soybean (U.S. Pat. Nos. 5,416,011; 5,569,834; 5,824,877; 5,563,04455 and 5,968,830); pineapple (U.S. Pat. No. 5,952,543); poplar (U.S. Pat. No. 4,795,855); monocots in general (U.S. Pat. Nos. 5,591,616 and 6,037,522); brassica (U.S. Pat. Nos. 5,188,958; 5,463,174 and 5,750,871); and cereals (U.S. Pat. No. 6,074,877). It will be understood by those skilled in the art that the above protocols may be adapted for example, for use with alternative selectable marker for transformation.

The plant-derived sequences in the vectors or minicircles of the invention may be derived from any plant species.

In one embodiment the plant-derived sequences in the vectors or minicircles of the invention are from gymnosperm species. Preferred gymnosperm genera include Cycas, Pseudotsuga, Pinus and Picea. Preferred gymnosperm species include Cycas rumphii, Pseudotsuga menziesii, Pinus radiata, Pinus taeda, Pinus pinaster, Picea engelmannia×sitchensis, Picea sitchensis and Picea glauca.

In a further embodiment the plant-derived sequences in the vectors or minicircles of the invention are from bryophyte species. Preferred bryophyte genera include Marchantia, Physcomitrella and Ceratodon. Preferred bryophyte species include Marchantia polyinorpha, Tortula ruralis, Physcomitrella patens and Ceratodon purpureous.

In a further embodiment the plant-derived sequences in the vectors or minicircles of the invention are from algae species. Preferred algae genera include Chlamydomonas. Preferred algae species include Chlamydomonas reinhardtii.

In a further embodiment the plant-derived sequences in the vectors or minicircles of the invention are from angiosperm species. Preferred angiosperm genera include Aegilops, Allium, Amborella, Anopterus, Apium, Arabidopsis, Arachis, Asparagus, Atropa, Avena, Beta, Betula, Brassica, Camellia, Capsicuin, Chenopodium, Cicer, Citrus, Citrullus, Coffea, Cucumis, Elaeis, Eschscholzia, Eucalyptus, Fagopyrum, Fragaria, Glycine, Gossypium, Helianthus, Hevea, Hordeum, Humulus, Ipomoea, Lactuca, Limonium, Linum, Lolium, Lotus, Lycopersicon, Lycoris, Malus, Manihot, Medicago, Mesembryanthemum, Musa, Nicotiana, Nuphar, Olea, Oryza, Persea, Petunia, Phaseolus, Pisum, Plumbago, Poncirus, Populus, Prunus, Puccinellia, Pyrus, Quintinia, Raphanus, Saccharum, Schedonorus, Secale, Sesamum, Solanum, Sorghum, Spinacia, Thellungiella, Theobroma, Triticum, Vaccinium, Vitis, Zea and Zinnia.

Preferred angiosperm species include Aegilops speltoides, Allium cepa, Amborella trichopoda, Anopterus macleayanus, Apium graveolens, Arabidopsis thaliana, Arachis hypogaea, Asparagus officinalis, Atropa belladonna, Avena sativa, Beta vulgaris, Brassica napus, Brassica rapa, Brassica oleracea, Capsicum annuum, Capsicum frutescens, Cicer arietinum, Citrullus lanatus, Citrus clementina, Citrus reticulata, Citrus sinensis, Coffea arabica, Coffea canephora, Cucumis sativus, Elaeis guineesis, Eschscholzia californica, Eucalyptus tereticornis, Fagopyrum esculentum, Fragaria×ananassa, Glycine max, Gossypium arboreum, Gossypium hirsutum, Gossypium raimondii, Helianthus annuus, Helianthus argophyllus, Hevea brasiliensis, Hordeum vulgare, Humulus lupulus, Ipomoea batatas, Ipomoea nil, Lactuca sativa, Limonium bicolor, Linum usitatissimum, Lolium multiflorum, Lotus corniculatus, Lycopersicon esculentum, Lycopersicon penellii, Lycoris longituba, Malus×domestica, Manihot esculenta, Medicago truncatula, Mesembryanthemum crystallinum, Nicotiana benthamiana, Nicotiana tabacum, Nuphar advena, Olea europea, Oryza sativa, Oryza minuta, Persea americana, Petunia hybrida, Phaseolus coccineus, Phaseolus vulgaris, Pisum sativum, Plumbago zeylanica, Poncirus trifoliata, Populus alba×tremula, Populus tremula×tremuloides, Populus tremula, Populus balsamifera×teldoides), Prunus americana, Prunus armeniaca, Prunus domestica, Prunus dulcis, Prunus persica, Puccinellia tenuiflora, Pyrus communis, Quintinia verdonii, Raphanus staivus, Saccharum officinarum, Schedonorus arundinaceus, Secale cereale, Sesamum indicum, Solanum habrochaites, Solanum lycopersicum, Solanum nigrum, Solanum tuberosum, Sorghum bicolor, Sorghum propinquum, Spinacia oleracea, Thellungiella halophila, Thellungiella salsuginea, Theobroma cacao, Triticum aestivum, Triticum durum, Triticum monococcum, Vaccinium corymbosum, Vitis vinifera, Zea mays and Zinnia elegans.

Particularly preferred angiosperm genera include Solanum, Petunia and Allium. Particularly preferred angiosperm species include Solanum tuberosum, Petunia hybrida and Allium cepa.

The plant cells and plants of the invention may be derived from any plant species.

In one embodiment the plant cells and plants of the invention are from gymnosperm species. Preferred gymnosperm genera include Cycas, Pseudotsuga, Pinus and Picea. Preferred gymnosperm species include Cycas rumphil, Pseudotsuga menziesii, Pinus radiata, Pinus taeda; Pinus pinaster, Picea engelmannia×sitchensis, Picea sitchensis and Picea glauca.

In a further embodiment the plant cells and plants of the invention are from bryophyte species. Preferred bryophyte genera include Marchantia, Tortula, Physcomitrella and Ceratodon. Preferred bryophyte species include Marchantia polymorpha, Tortula ruralis, Physcomitrella patens and Ceratodon purpureous.

In a further embodiment the plant cells and plants of the invention are from algae species. Preferred algae genera include Chlamydomonas. Preferred algae species include Chlamydomonas reinhardtii.

In a further embodiment the plant cells and plants of the invention are from angiosperm species. Preferred angiosperm genera include Aegilops, Allium, Amborella, Anopterus, Apium, Arabidopsis, Arachis, Asparagus, Atropa, Avena, Beta, Betula, Brassica, Camellia, Capsicum, Chenopodium, Cicer, Citrus, Citrullus, Coffea, Cucumis, Elaeis, Eschscholzia, Eucalyptus, Fagopyrum, Fragaria, Glycine, Gossypium, Helianthus, Hevea, Hordeum, Humulus, Ipomoea, Lactuca, Limonium, Linum, Lolium, Lotus, Lycopersicon, Lycoris, Malus, Manihot, Medicago, Mesembryanthemum, Musa, Nicotiana, Nuphar, Olea, Oryza, Persea, Petunia, Phaseolus, Pisum, Plumbago, Poncirus, Populus, Prunus, Puccinellia, Pyrus, Quintinia, Raphanus, Saccharum, Schedonorus, Secale, Sesamum, Solanum, Sorghum, Spinacia, Thellungiella, Theobroma, Triticum, Vaccinium, Vitis, Zea and Zinnia.

Preferred angiosperm species include Aegilops speltoides, Allium cepa, Amborella trichopoda, Anopterus macleayanus, Apium graveolens, Arabidopsis thaliana, Arachis hypogaea, Asparagus officinalis, Atropa belladonna, Avena sativa, Beta vulgaris, Brassica napus, Brassica rapa, Brassica oleracea, Capsicum annuum, Capsicum frutescens, Cicer arietinum, Citrullus lanatus, Citrus clementina, Citrus reticulata, Citrus sinensis, Coffea arabica, Coffea canephora, Cucumis sativus, Elaeis guineesis, Eschscholzia californica, Eucalyptus tereticornis, Fagopyrum esculentum, Fragaria×ananassa, Glycine max, Gossypium arboreum, Gossypium hirsutum, Gossypium raimondii, Helianthus annuus, Helianthus argophyllus, Hevea brasiliensis, Hordeum vulgare, Humulus lupulus, Ipomoea batatas, Ipomoea nil, Lactuca saliva, Limonium bicolor, Linum usitatissimum, Lolium multifiorum, Lotus corniculatus, Lycopersicon esculentum, Lycopersicon penellii, Lycoris longituba, Malus×domestica, Manihot esculenta, Medicago truncatula, Mesembryanthemum crystallinum, Nicotiana benthamiana, Nicotiana tabacum, Nuphar advena, Olea europea, Oryza sativa, Oryza minuta, Persea americana, Petunia hybrida, Phaseolus coccineus, Phaseolus vulgaris, Pisum sativum, Plumbago zeylanica, Poncirus trifoliata, Populus alba×tremula, Populus tremula×tremuloides, Populus tremula, Populus balsamifera×teldoides), Prunus americana, Prunus armeniaca, Prunus domestica, Prunus dulcis, Prunus persica, Puccinellia tenuiflora, Pyrus communis, Quintinia verdonii, Raphanus staivus, Saccharum officinarum, Schedonorus arundinaceus, Secale cereale, Sesamum indicum, Solanum habrochaites, Solanum lycopersicum, Solanum nigrum, Solanum tuberosum, Sorghum bicolor, Sorghum propinquum, Spinacia oleracea, Thellungiella halophila, Thellungiella salsuginea, Theobroma cacao, Triticum aestivum, Triticum durum, Triticum monococcum, Vaccinium corymbosum, Vitis vinifera, Zea mays and Zinnia elegans.

Particularly preferred angiosperm genera include Solanum, Petunia and Allium. Particularly preferred angiosperm species include Solanum tuberosum, Petunia hybrida and Allium cepa.

The cells and plants of the invention may be grown in culture, in greenhouses or the field. They may be propagated vegetatively, as well as either selfed or crossed with a different plant strain and the resulting hybrids, with the desired phenotypic characteristics, may be identified. Two or more generations may be grown to ensure that the subject phenotypic characteristics are stably maintained and inherited. Plants resulting from such standard breeding approaches also form an aspect of the present invention.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### BRIEF DESCRIPTION OF THE DRAWINGS

FIG. 1 shows a plasmid map of pUC57PhMCcab.

FIG. 2 shows a plasmid map of pUC57PhMCcabDP.

FIG. 3 shows a plasmid map of pUC57PhMCcabPH.

FIG. 4 shows the plasmid backbone generated following Cre-induced intramolecular recombination of pUC57PhMCcabDP and pUC57PhMCcabPH.

FIG. 5 shows the petunia-derived ‘Deep purple’ minicircle generated following Cre-induced intramolecular recombination of pUC57PhMCcabDP.

FIG. 6 shows the petunia-derived ‘Purple Haze’ minicircle generated following Cre-induced intramolecular recombination of pUC57PhMCcabPH.

FIG. 7 shows the induction of petunia minicircles from pUC57PhMCcabDP. Escherichia coli strain 294-Cre with pUC57PhMCcabDP was cultured overnight on a shaker at 28° C. in liquid LB medium with 100 mg/l ampillicin, then transferred to 37° C. for 0-5 hours for induction of Cre recombinase expression. All lanes are loaded with 5 μl DNA purified using a Roche Miniprep Kit. Lane 1, 2 log ladder (NEB, Beverly, Mass., USA); lane 2, uninduced culture maintained at 28° C. with only the 5715 bp pUC57PhMCcabDP plasmid; lanes 3-6, induced cultures after 1, 2, 3, and 5 hours respectively at 37° C. with diminishing amounts of the 5715 bp pUC57PhMCcabDP plasmid and increasing yields of both the 3443 bp recombination backbone plasmid and the 2272 bp petunia ‘Deep Purple’ minicircle; lane 7, 1 hour induction at 37° C. followed by a further 2 hours at 28° C.

FIG. 8 shows the induction of petunia minicircles from pUC57PhMCcabPH. Escherichia coli strain 294-Cre with pUC57PhMCcabPH was cultured overnight on a shaker at 28° C. in liquid LB medium with 100 mg/l ampillicin, then transferred to 37° C. for 0-5 hours for induction of Cre recombinase expression. All lanes are loaded with 5 μl DNA purified using a Roche Miniprep Kit. Lane 1, uninduced culture maintained at 28° C. with only the 5697 bp pUC57PhMCcabPH plasmid; lanes 2-5, induced cultures after 1, 2, 3, and 5 hours respectively at 37° C. with diminishing amounts of the 5697 bp pUC57PhMCcabPH plasmid and increasing yields of both the 3443 bp recombination backbone plasmid and the 2254 bp petunia ‘Purple Haze’ minicircle; lane 6, 1 hour induction at 37° C. followed by a further 2 hours at 28° C.; lane 7, 2 hour induction at 37° C. followed by a further 2 hours at 28° C.; lane 8, 2 log ladder (NEB, Beverly, Mass., USA).

FIG. 9 shows the purification of the intact 2272 bp circular petunia ‘Deep Purple’ minicircle. An overnight culture of Escherichia coli strain 294-Cre with pUC57PhMCcabDP grown at 28° C. in liquid LB medium with 100 mg/l ampillicin was transferred to 37° C. for 6 hours to induce Cre expression and recombination. Lane 1, the GeneRuler DNA ladder mix #SM0331 (Fermentas, Hanover, Md., USA) size marker; lanes 2-4, purified DNA restricted with BamHI and EcoRI to yield linearised fragments from the 3443 bp pUC57-based backbone plasmid and any remaining pUC57PhMCcabDP plasmid, plus the intact 2272 bp circular petunia minicircle; lanes 5-7, purified DNA was restricted with BamHI and EcoRI and linearised plasmid digested with λ Exonuclease leaving only the intact 2272 bp circular petunia ‘Deep Purple’ minicircle.

FIG. 10 shows the purification of the intact 2258 bp circular petunia ‘Purple Haze’ minicircle. An overnight culture of Escherichia coli strain 294-Cre with pUC57PhMCcabPH grown at 28° C. in liquid LB medium with 100 mg/l ampillicin was transferred to 37° C. for 6 hours to induce Cre expression and recombination. Lanes 1-3, purified DNA restricted with BamHI and EcoRI to yield linearised fragments from the 3443 bp pUC57-based backbone plasmid and any remaining pUC57PhMCcabDP plasmid, plus the intact 2254 bp circular petunia minicircle; lanes 4-6, purified DNA was restricted with BamHI and EcoRI and linearised plasmid digested with λ Exonuclease leaving only the intact 2254 bp circular petunia ‘Purple Haze’ minicircle. Lane 7, the GeneRuler DNA ladder mix #SM0331 (Fermentas, Hanover, Md., USA) size marker.

FIG. 11 shows the red pigmentation in vegetative tissue of petunia following bombardment with the petunia ‘Deep Purple’ minicircle. Upper, development of red pigmentation in a leaf segment of Petunia hybrida genotype ‘V30’ seven days following bombardment with the ‘Deep Purple’ minicircle; lower, shoot primordia regeneration of Petunia hybrida genotype ‘Mitchell’ with red pigmentation three weeks following bombardment with the ‘Deep Purple’ minicircle.

FIG. 12 shows the red pigmentation in vegetative tissue of petunia following bombardment with the petunia ‘Purple Haze’ minicircle. Upper, development of red pigmentation in a leaf segment of Petunia hybrida genotype ‘V30’ seven days following bombardment with the ‘Purple Haze’ minicircle; lower, shoot regeneration of Petunia hybrida genotype ‘Mitchell’ with red pigmentation three weeks following bombardment with the ‘Purple Haze’ minicircle.

FIG. 13 shows a plasmid map of pUC57StMCpatStan2.

FIG. 14 shows the plasmid backbone generated following FLP-induced intramolecular recombination of pUC57StMCpatStan2.

FIG. 15 shows the potato-derived ‘patStan2’ minicircle generated following FLP-induced intramolecular recombination of pUC57StMCpatStan2.

FIG. 16 shows a plasmid map of pPOTLOXP2:Stan2 GBSSPT.

FIG. 17 shows a plasmid map of pPOTLOXP2:Stan2 Patatin.

FIG. 18 shows a plasmid backbone generated following Cre-induced intramolecular recombination of pPOTLOXP2:Stan2 GBSSPT and pPOTLOXP2:Stan2 Patatin.

FIG. 19 shows the potato-derived ‘Stan2 GBSSMC’ minicircle generated following Cre-induced intramolecular recombination of pPOTLOXP2:Stan2 GBSSPT.

FIG. 20 shows the potato-derived ‘Stan2 PatatinMC’ minicircle generated following Cre-induced intramolecular recombination of pPOTLOXP2:Stan2 Patatin.

FIG. 21 shows the induction of potato minicircles from pPOTLOXP2:Stan2 GBSSPT and pPOTLOXP2:Stan2 Patatin. Escherichia coli strain 294-Cre with pPOTLOXP2:Stan2 GBSSPT or pPOTLOXP2:Stan2 Patatin was cultured overnight on a shaker at 28° C. in liquid LB medium with 100 mg/l ampillicin, then transferred to 37° C. for 4 hours for induction of Cre recombinase expression. All lanes are loaded with 5 μl DNA purified using an Invitrogen PureLink Quick Plasmid Miniprep Kit and digested with HindIII. Lane 1, Hyperladder I (Bioline, Taunton, Mass., USA); lanes 2 and 4, uninduced cultures of independent clones with pPOTLOXP2:Stan2 GBSSPT maintained at 28° C. with the expected 6563 bp and 1015 bp fragments; lanes 3 and 5, induced cultures of independent clones at 37° C. with substantially reduced amounts of the pPOTLOXP2:Stan2 GBSSPT fragments, and high yields of both the 4472 bp recombination backbone plasmid and the 3106 bp potato ‘Stan2 GBSSMC’ minicircle; lanes 5 and 7, uninduced cultures of independent clones with pPOTLOXP2:Stan2 Patatin maintained at 28° C. with the expected 6492 bp and 1015 bp fragments; lanes 3 and 5, induced cultures of independent clones at 37° C. with substantially reduced amounts of the pPOTLOXP2:Stan2 Patatin fragments, and high yields of both the 4472 bp recombination backbone plasmid and the 3035 bp potato ‘Stan2 PatatinMC’ minicircle.

FIG. 22 shows the design of a minicircle generating T-DNA for Agrobacterium-mediated gene transfer. This represents a 4599 bp fragment flanked by SalI restriction enzyme recognition sites cloned onto the 8235 bp backbone of the binary vector pART27MCS.

FIG. 23 shows the plasmid pBAD202DtopoCre.

FIG. 24 shows the minicircle derived from pMOA38 upon arabinose induction.

FIG. 25 shows the arabinose induction of T-DNA minicircles from pMOA38 in Escherichia coli DH5α. Plasmid preparations from overnight cultures in LB medium with and without 0.2-20% L-arabinose were restricted with BamHI. Lane 1, the GeneRuler DNA ladder mix #SM0331 (Fermentas, Hanover, Md.) size marker; lane 2, uninduced culture; lane 3, induced with 20% L-arabinose; lane 4, induced with 2% L-arabinose; lane 5, induced with 0.2% L-arabinose. The presence of a 1916 bp fragment in lanes 3 and 4 is diagnostic for the formation of the minicircle.

FIG. 26 shows the DNA sequence from transformed plants across the Cre recombinase-induced intramolecular recombination event to form the minicircle from pMOA38. The DNA sequence is presented from PCR products from seven transformed tobacco plants (JNT02-3, JNT02-8, JNT02-9, JNT02-18, JNT02-22, JNT02-28 and JNT02-55) and aligned with the expected sequence from the minicircle and the sequence surrounding the loxP66 and loxP71 sites in pMOA38. The core LoxP sequence in common between loxP66 and loxP71 is highlighted.

FIG. 27 shows the design of a minicircle generating T-DNA for Agrobacterium-mediated gene transfer. This represents a 4586 bp fragment flanked by SalI restriction enzyme recognition sites cloned onto the 8235 bp backbone of the binary vector pART27MCS.

FIG. 28 shows the minicircle derived from pMOA40 upon arabinose induction.

FIG. 29 shows the arabinose induction of T-DNA minicircles from pMOA40 in Escherichia coli DH5α. Plasmid preparations from overnight cultures in LB medium with and without 0.2-20% L-arabinose or D-arabinose were restricted with BamHI. Lanes 1 and 9, the GeneRuler DNA ladder mix #SM0331 (Fermentas, Hanover, Md.) size marker; lane 2, uninduced culture; lane 3, induced with 20% L-arabinose; lane 4, induced with 2% L-arabinose; lane 5, induced with 0.2% L-arabinose; lane 6, induced with 20% D-arabinose; lane 7, induced with 2% D-arabinose; lane 8, induced with 0.2% D-arabinose. The presence of a 1918 bp fragment in lanes 3 and 4 is diagnostic for the formation of the minicircle.

FIG. 30 shows the DNA sequence from transformed plants across the Cre recombinase-induced intramolecular recombination event to form the minicircle from pMOA40. The DNA sequence is presented from PCR products from fourteen independently derived transformed tobacco plants (S1-01, S1-05, JNT01-05, JNT01-09, JNT01-20, JNT01-22, JNT01-25, JNT01-26, JNT01-27, JNT01-29, JNT01-30, JNT01-35, JNT01-39, and JNT01-44) and aligned with the expected sequence from the minicircle and the sequence surrounding the loxP66 and loxP71 sites in pMOA40. The core LoxP sequence in common between loxP66 and loxP71 is highlighted.

FIG. 31 shows the design of a 2713 bp intragenic potato-derived minicircle generating a T-DNA for Agrobacterium-mediated gene transfer.

FIG. 32 shows the plasmid pGreenII-MCS.

FIG. 33 shows the pPOTIV10 T-DNA region with CodA negative selection marker gene that generates an intragenic potato-derived T-DNA for Agrobacterium-mediated gene transfer.

FIG. 34 shows the plasmid pSOUPLacFLP.

FIG. 35 shows the minicircle derived from pPOTIV10 upon FLP induction.

FIG. 36 shows the design of a 2903 bp intragenic potato-derived minicircle producing a T-DNA with a selectable marker for chlosulfuron tolerance for Agrobacterium-mediated gene transfer.

FIG. 37 shows the plasmid pSOUParaBADCre.

FIG. 38 shows the minicircle derived from pPOTIV11 upon Cre induction.

### EXAMPLES

The invention will now be illustrated with reference to the following non-limiting examples.

Examples 1 and 2 describe compositions and methods for transformation via direct DNA uptake. Example 1 involves use of a loxP-like/Cre recombination system. Example 2 involves use of a frt-like/FLP recombination system and a loxP-like/Cre recombination system.

Examples 3 and 4 describes compositions and methods for transformation via Agrobacterium-mediated gene transfer. Example 3 involves use of a loxP-like/Cre recombination system. Example 4 involves use of a frt-like/FLP recombination system and a loxP-like/Cre recombination system.

Example 5 describes design construction and verification of plant-derived loxP-like recombinase recognition sequences.

Example 6 describes design construction and verification of plant-derived frt-like recombinase recognition sequences.

### Example 1

### Design, Construction, Production and Use of Petunia Minicircles for Direct DNA Uptake

A 2129 bp sequence of DNA composed from a series of DNA fragments derived from petunia

(Petunia hybrida) was constructed. A key component was a 0.7 kb direct repeat produced by adjoining two EST's to create a petunia-derived loxP site at their junction. A petunia gene expression cassette, consisting of the 5′ promoter and 3′ terminator regulatory regions of the petunia cab 22R gene, was positioned between these direct repeats. The cloning of this 2129 bp fragment into a standard bacterial plasmid allows the in vivo generation of petunia-derived minicircles by site-specific intramolecular recombination upon inducible expression of the Cre recombinase enzyme in bacteria such as Escherichia coli. The resulting minicircle is composed entirely of DNA derived from petunia. The cloning of the coding regions of petunia genes between the regulatory regions of the cab 22R gene provides a tool to generate DNA molecules for delivery of chimeric petunia genes by transformation to plants such as petunia. In this manner genes can be transformed in plants without foreign DNA and without the undesirable plasmid backbone sequences.

A 2136 bp sequence composed of the above petunia-derived sequence, flanked by a few nucleotides at each end to generate useful PmeI and HpaI restriction sites, was synthesised by Genscript Corporation (Piscatawa, N.J., USA, www.genscript.com) and cloned into pUC57. All plasmid constructions were performed using standard molecular biology techniques of plasmid isolation, restriction, ligation and transformation into Escherichia coli strain DH5α (Sambrook et al. 1987, Molecular Cloning: A Laboratory Manual, 2″ ed., Cold Spring Harbor Press), unless otherwise stated.

The resulting plasmid was designated pUC57PhMCcab. The full sequence of pUC57PhMCcab is shown in SEQ ID NO: 1, where:
nucleotides 1-359 are from the pUC57 vector;
nucleotides 360-363 are added to create a PmeI restriction site as a option for future cloning;
nucleotides 364-1075 represent a petunia-derived DNA sequence composed of two adjoining two EST's (nucleotides 364-827 originating from SGN-E526158 nucleotides 99-562; nucleotides 828-1075 originating from the reverse complement of SGN-E528397 nucleotides 7-254) to create a loxP site from nucleotides 816-840;
nucleotides 1076-1615 are from the Cab 22R promoter (Gidoni et al. 1989, Molecular and General Genetics, 215: 337-344);
nucleotides 1613-1618 create a SpeI restriction site
nucleotides 1616-1762 are from the Cab 22R terminator sequence (Dunsmuir 1985, Nucleic Acids Research, 13: 2503-2518; nucleotides 1035-1181 of NCBI accession X02360);
nucleotides 1760-2492 represent a petunia-derived DNA sequence composed of two adjoining two EST's (nucleotides 1763-2240 originating from SGN-E526158 nucleotides 85-562; nucleotides 2241-2492 originating from the reverse complement of SGN-E528397 nucleotides 3-254) to create a loxP site from nucleotides 2229-2253;
nucleotides 2493-2495 are added to create a HpaI restriction site as a option for future cloning; and
nucleotides 2496-4856 are from the pUC57 vector.

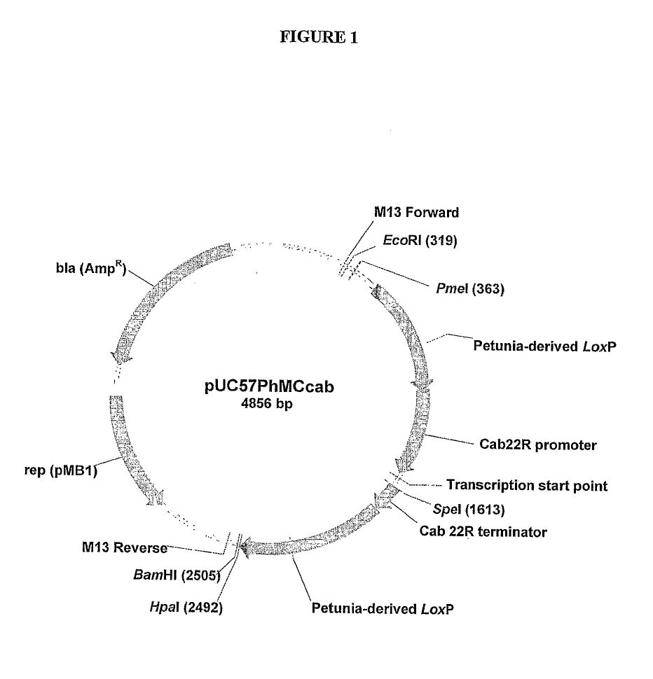

A plasmid map of pUC57PhMCcab is illustrated in FIG. 1. The region from nucleotides 364-2492 is composed entirely of DNA sequences derived from petunia and has been verified by DNA sequencing between the M13 forward and M13 reverse universal primers.

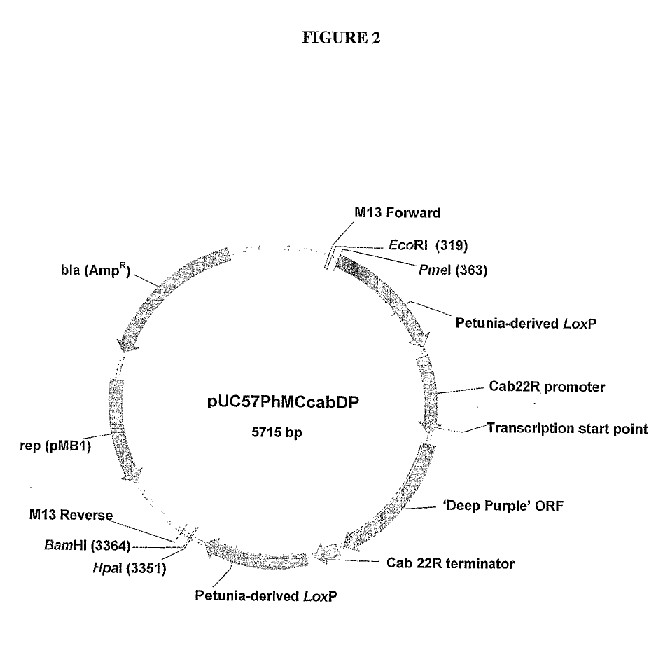

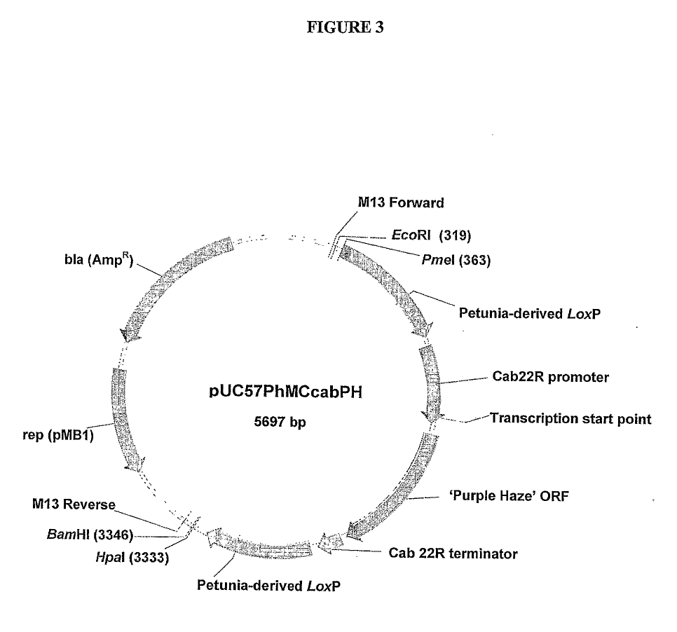

The 859 bp coding region (including the 5′ and 3′ untranslated sequences) of a myb transcription factor ‘Deep Purple’ (from Plant & Food Research) and the 841 bp coding region (including the 5′ and 3′ untranslated sequences) of a myb transcription factor ‘Purple Haze’ (from Plant & Food Research) were then independently cloned into the SpeI site between the promoter and 3′ terminator of the Cab 22R gene. This was achieved blunt ligations following treatment of the fragments with Quick Blunting Kit (NEB, Beverly, Mass., USA). The resulting plasmids, pUC57PhMCcabDP and pUC57PhMCcabPH, are illustrated in FIG. 2 and FIG. 3 respectively.

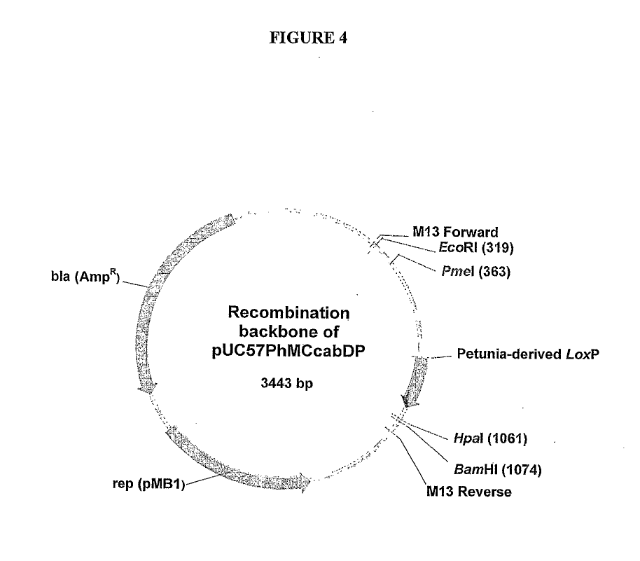

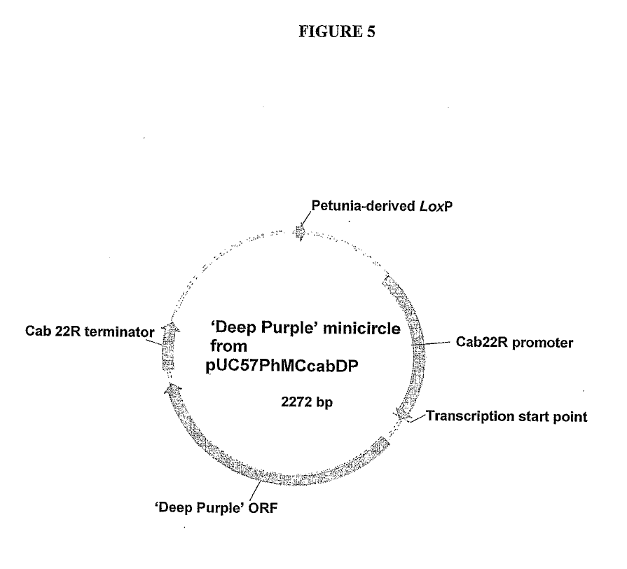

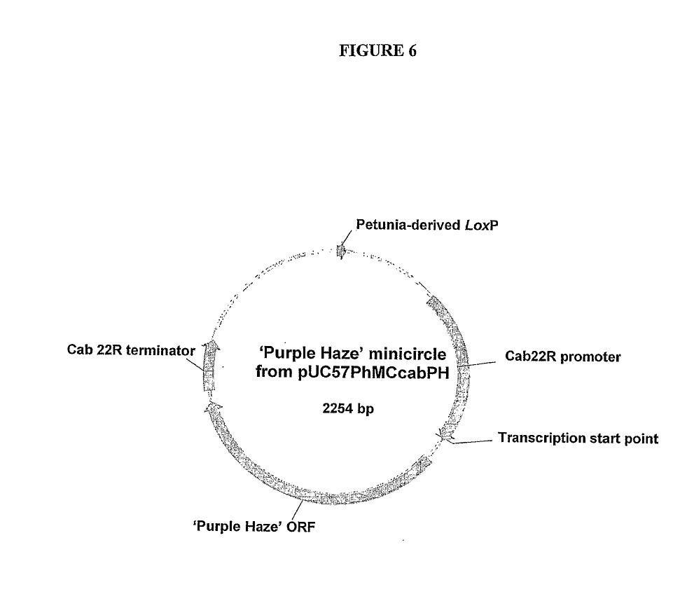

The ability for pUC57PhMCcabDP and pUC57PhMCcabPH to generate minicircles by intramolecular recombination between the petunia-derived LoxP sites was tested in vivo using Escherichia coli strain 294-Cre with Cre recombinase under the control of the heat inducible λPr promoter (Buchholz et al. 1996, Nucleic Acids Research, 24: 3118-3119). The pUC57PhMCcabDP and pUC57PhMCcabPH plasmids were independently transformed into E. coli strain 294-Cre and maintained by selection in LB medium with 100 mg/l ampillicin and incubation at 28° C. Raising the temperature to 37° C. induced the expression of Cre recombinase in E. coli strain 294-Cre, resulting in recombination between the two petunia-derived LoxP sites. For pUC57PhMCcabDP this produced a 3443 bp plasmid derived from the pUC57 sequence with a short region of petunia DNA (FIG. 4) and the 2272 bp petunia minicircle ‘Deep Purple’ (FIG. 5). For pUC57PhMCcabPH this produced the same 3443 bp plasmid derived from the pUC57 sequence with a short region of petunia DNA (FIG. 4) and the 2254 bp petunia minicircle ‘Purple Haze’ (FIG. 6).

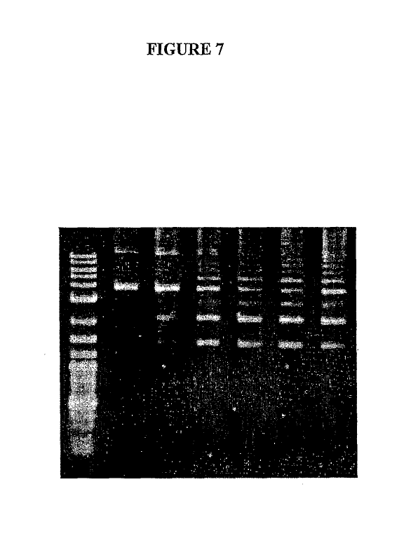

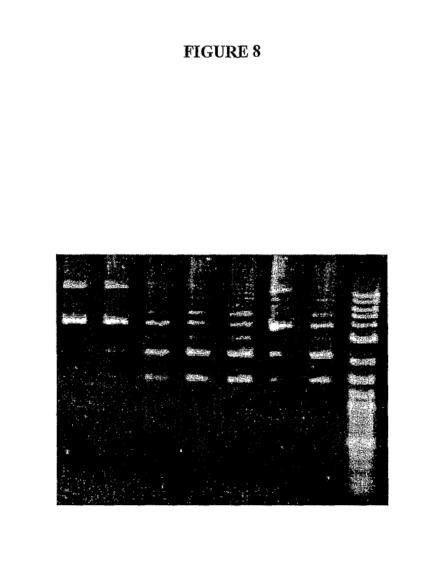

When cultured overnight at 28° C. with uninduced Cre recombinase only the 5715 bp pUC57PhMCcabDP plasmid (FIG. 7, lane 2) or the 5697 bp pUC57PhMCcabPH plasmid (FIG. 8, lane 1) was present. After 1 hour induction at 37° C. the presence of both the 3443 bp recombination backbone plasmid and the 2272 bp petunia ‘Deep Purple’ minicircle (FIG. 7, lane 3) or the 2254 bp petunia ‘Purple Haze’ minicircle (FIG. 8, lane 2) were evident. The yield of these recombination products increased with 2-5 hours induction at 37° C. (FIG. 7, lanes 4-6; FIG. 8, lanes 3-5). Higher yields of recombination products were also evident after only 1-2 hours induction at 37° C. followed by a further 2 hours at 28° C. (FIG. 7, lane 7; FIG. 8, lanes 6-7), indicating that the Cre recombinase enzyme was still active over time without continual induction.

To produce larger quantities of petunia minicircles for plant transformation several 50 ml cultures of E. coli strain 294-Cre with pUC57PhMCcabDP or pUC57PhMCcabPH were cultured overnight on a shaker at 28° C. in liquid LB medium with 100 mg/l ampillicin. After overnight growth, the cultures were transferred to 37° C. to induce Cre expression and recombination. After 6 hours at 37° C., the cultures were centrifuged at 4,000 rpm for 20 minutes and the well-drained pellets of E. coli cells were stored at −20° C. for subsequent DNA purification by alkaline lysis and ethanol precipitation (Sambrook et al. 1987, Molecular Cloning: A Laboratory Manual, 2^{nd} ed., Cold Spring Harbor Press). The DNA pellets were completely dried, then dissolved in 500 μl TE (pH 8.0) plus 100 μg/ml RNase A.

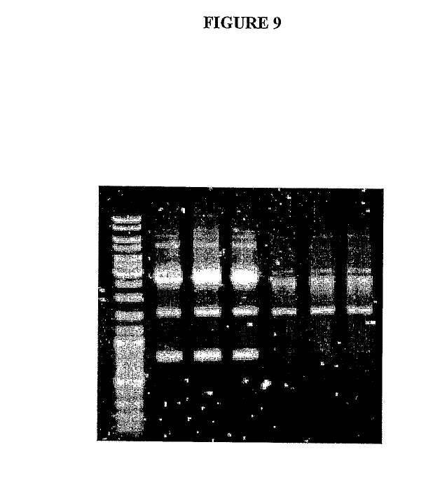

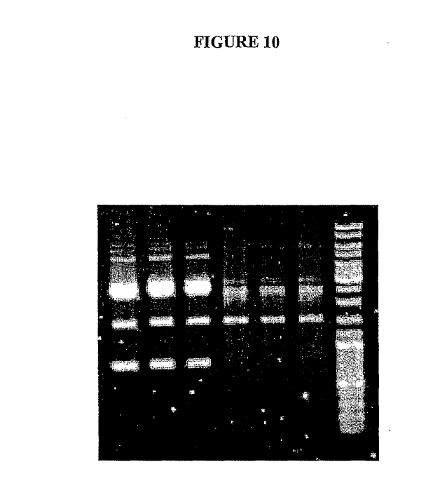

The DNA was then restricted overnight at 37° C. with BamHI and EcoRI to linearise the 3443 bp UC57-based backbone plasmid (see FIG. 4) and any remaining pUC57PhMCcabDP plasmid (see FIG. 2) or pUC57PhMCcabPH plasmid (see FIG. 3), but leaving the 2272 bp circular petunia ‘Deep Purple’ minicircle (see FIG. 5) or the 2254 bp circular petunia ‘Purple Haze’ minicircle (see FIG. 6) intact. Following restriction, DNA was passed through Qiagen PCR purification columns and eluted with 50 μl of distilled H₂O. The purified digests were then treated with λ Exonuclease (NEB MO262S) following the manufacturer's guidelines and incubated at 37° C. for 4 hours to digest the linear DNA. The exonuclease was then heat inactivated at 72° C. for 10 minutes. The samples were purified by passing through Qiagen PCR purification columns and eluted with 50 μl of distilled H₂O to yield the remaining intact 2272 bp circular petunia minicircle ‘Deep Purple’ (FIG. 9) or the remaining intact 2254 bp circular petunia minicircle ‘Deep Purple’ (FIG. 10).

The purified ‘Deep Purple’ minicircle is composed entirely of DNA fragments derived from petunia and contains a chimeric gene anticipated to induce the biosynthesis of anthocyanins (FIG. 5). The full sequence of the ‘Deep Purple’ minicircle is shown in SEQ ID NO: 2, where:
nucleotides 1-12 originate from SGN-E526158 nucleotides 551-562;
nucleotides 13-260 originate from the reverse complement of SGN-E528397 nucleotides 7-254;
nucleotides 1-25 represent a petunia-derived loxP site;
nucleotides 261-802 are from the Cab 22R promoter (Gidoni et al. 1989, Molecular and General Genetics, 215: 337-344);
nucleotides 803-1661 represent the coding region of a myb transcription factor ‘Deep Purple’ from Plant & Food Research;
nucleotides 1662-1806 are from the Cab 22R terminator sequence (Dunsmuir 1985, Nucleic Acids Research, 13: 2503-2518; nucleotides 1037-1181 of NCBI accession X02360); and
nucleotides 1807-2272 originate from SGN-E526158 nucleotides 85-550.

The purified 2258 bp ‘Purple Haze’ minicircle is composed entirely of DNA fragments derived from petunia and contains a chimeric gene anticipated to induce the biosynthesis of anthocyanins (FIG. 6). The full sequence of the ‘Purple Haze’ minicircle is shown in SEQ ID NO: 3, where:
nucleotides 1-12 originate from SGN-E526158 nucleotides 551-562;
nucleotides 13-260 originate from the reverse complement of SGN-E528397 nucleotides 7-254;
nucleotides 1-25 represent a petunia-derived loxP site;
nucleotides 261-802 are from the Cab 22R promoter (Gidoni et al. 1989, Molecular and General Genetics, 215: 337-344);
nucleotides 803-1643 represent the coding region of a myb transcription factor ‘Purple Haze’ from Plant & Food Research;
nucleotides 1644-1788 are from the Cab 22R terminator sequence (Dunsmuir 1985, Nucleic Acids Research, 13: 2503-2518; nucleotides 1037-1181 of NCBI accession X02360); and
nucleotides 1789-2254 originate from SGN-E526158 nucleotides 85-550.

Petunia plants were transformed with the 2272 bp petunia ‘Deep purple’ minicircle DNA or the 2254 bp petunia ‘Purple Haze’ minicircle DNA using standard biolistic transformation methods. Since the minicircles each contain a petunia Myb gene under the transcriptional control of the regulatory regions of the petunia cab 22R gene, the resulting induction of anthocyanin biosynthesis provides enhanced pigmentation in vegetative tissue to enable the visual selection of transformed tissue.

Young leaf pieces were harvested from greenhouse-grown petunia plants (genotypes Mitchell and V30) and surface-sterilised by immersion with gentle shaking for 10 minutes in 10% commercial bleach (1.5% sodium hypochlorite) containing a few drops of 1% Tween 20, followed by several washes with sterile distilled water. A biolistic gold preparation was then made using a standard protocol: 1 μg of minicircle DNA, 20 μl of 0.1 M spermidine and 50 μl of 2.5 M CaCl₂ were mixed with a suspension containing 50 mg of sterile 1.0 μm diameter gold particles to give a total volume of 130 μl. After 5 minutes 95 μl of supernatant was discarded leaving 35 μl of DNA-bound gold suspension.

The leaf pieces were then bombarded using a particle inflow gun. Each leaf piece was bombarded twice with 5 μl of the gold suspension. After bombardment the leaf pieces were cut into small sections (approximately 5 mm²) and transferred to shoot regeneration medium consisting of MS salts (Murashige and Skoog 1962, Physiologia Plantarum, 15: 473-497), B5 vitamins (Gamborg et al. 1968, Experimental Cell Research, 50: 151-158), 3% sucrose, 3 mg/1 BAP, 0.2 mg/l IAA and 0.7% agar at pH 5.8. These were cultured at 25° C. under cool white fluorescent lamps (70-90 μmol m⁻²s⁻¹; 16-h photoperiod).

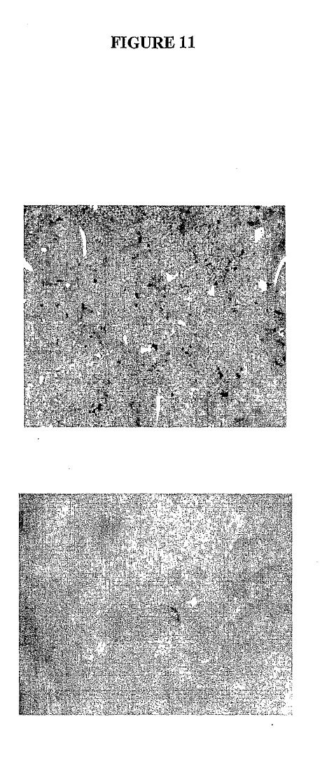

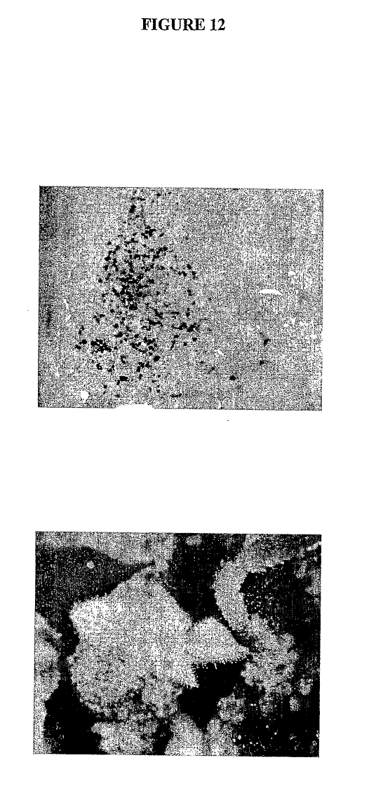

Red pigmented regions were visible on the surface of the leaf segments after 3 days and further intensified by day 7 for both the ‘Deep Purple’ minicircle (FIG. 11, upper) and the ‘Purple Haze’ minicircle (FIG. 12, upper). These developed into pigmented shoot primordia and regenerated complete shoots over the following three weeks (FIG. 11, lower; FIG. 12, lower). Shoots exhibiting red pigmentation in their vegetative tissue were then excised, dipped in a sterile solution of 100 mg/l IAA and transferred to the above medium without plant growth regulators (MS salts, B5 vitamins, 3% sucrose). After 3-4 weeks plants with roots were transferred to the greenhouse.

For the genotype petunia Mitchell transformed with the 2272 bp petunia ‘Deep Purple’ minicircle DNA, RNA was isolated from the shot zone 15 days after biolistic transformation. Leaf tissue was frozen in liquid nitrogen and ground to a powder. For 1 g of leaf tissue, one volume of GNTC (4M guanidine thiocyanate, 25 mM sodium citrate, 0.5% sodium lauryl sarcosinate, pH 7.0, with 8 μl/ml 2-mercaptoethanol added just prior to use), 0.1 volume 2M NaOAc at pH4, and one volume of phenol were added and thoroughly mixed by vortexing. Then 0.3 volume of chloroform:isoamyl alcohol (49:1) was added and thoroughly mixed by vortexing again, followed by centrifugation at 12000 rpm for 15 min at 4° C. The aqueous phase (500 μl) was collected and the RNA was precipitated with one volume cold isopropanol. After centrifugation at 14000 rpm for 15 min at 4° C., the supernatant was decanted off and pellet washed with 300 μl 70% ethanol. The pellet was dissolved in 30 μl sterile water.

RT-PCR was performed using the primers NA34 For (^{5′}ggggtacCATGAATACTTCTGTTTTTACGTC^{3′}—SEQ ID NO: 60) and PETCABPTRev (^{5′}GCCATCAAACAACCCGATAA^{3′}—SEQ ID NO: 61) which produce an expected product of 877 bp bridging the ‘Deep Purple’ coding region and the 3′ terminator sequence of the petunia Cab 22R gene. This transcription product is from a chimeric petunia gene it is only expected from tissue transformed with the petunia ‘Deep Purple’ minicircle and not from wild-type petunia. First strand cDNA was synthesised using SuperScript™ II Reverse Transcriptase (Invitrogen, Carlsbad, Calif.) according to manufacturer's instruction. RT-PCR was carried out in a DNA engine Thermal Cycler (Bio-Rad, California, USA). The reaction included 1 μl Taq DNA polymerase (5U/μl; Roche, Mannheim, Germany), 2 μl 10×PCR reaction buffer with MgCl₂ (Roche), 0.5 μl of dNTP mix (10 mM of each dNTP), 0.5 μl of each primer (at 10 μM), 5 μl of cDNA or RNA (50-100 ng) and water to total volume of 20 μl. The conditions for RT-PCR were: 2 min at 94° C. (to denature the SuperScript™ II RT enzyme), 35 cycles of 30 s 94° C., 30 s 50° C., 30 s 72° C. (PCR amplification), followed by 2 min extension at 72° C., then holding the reaction at 14° C. Amplified products were separated by electrophoresis in a 2% agarose gel and visualized under UV light after staining with ethidium bromide. Two PCR negative controls were used: RNA isolated from the shot zone (from which the cDNA was made) and cDNA from wild type petunia leaves shot with only gold particles. The cDNA from the shot zone yielded a band of the predicted 877 bp size. No such band was observed in either of the two negative controls, showing that the positive result was from the cDNA sample and not from non-integrated DNA from the shot event or from an endogenous gene product.

### Example 2

### Design, Construction, Production and Use of Potato Minicircles for Direct DNA Uptake

(A) Potato Minicircles Based on Potato-Derived frt-Like Sites

A 2960 bp sequence of DNA composed from a series of DNA fragments derived from potato (Solanum tuberosum) was constructed in silico. A key component was a direct repeat of about 0.35 kb produced by adjoining two EST's to create a potato-derived frt-like site at their junction. A chimeric potato gene, consisting of the coding region of a potato myb transcription factor, the D locus allele Stan2⁷⁷⁷ (Jung et al. 2009, Theoretical and Applied Genetics, 120: 45-57), under the transcriptional control of the regulatory regions of a potato patatin class I gene, was positioned between these direct repeats. The cloning of this 2960 bp fragment into a standard bacterial plasmid allows the in vivo generation of potato-derived minicircles by site-specific intramolecular recombination upon inducible expression of the FLP recombinase enzyme in bacteria such as Escherichia coli. The resulting minicircle is composed entirely of DNA fragments derived from potato with a chimeric gene to induce the biosynthesis of anthocyanins upon transformation of plants such as potato.

A 2966 bp sequence composed of the above potato-derived sequence, flanked by a few nucleotides at each end to generate useful SmaI restriction sites, was synthesised by Genscript Corporation (Piscatawa, N.J., www.genscript.com) and cloned into pUC57. All plasmid constructions were performed using standard molecular biology techniques of plasmid isolation, restriction, ligation and transformation into Escherichia coli strain DH5α, unless otherwise stated (Sambrook et al. 1987, Molecular Cloning: A Laboratory Manual, 2^{nd} ed., Cold Spring Harbor Press).

The resulting plasmid was designated pUC57StMCpatStan2. The full sequence of pUC57StMCpatStan2 is shown in SEQ ID NO:4; where:
nucleotides 1-413 are from the pUC57 vector;
nucleotides 414-416 are added to create a SmaI restriction site as a option for future cloning;
nucleotides 417-746 represent a potato-derived DNA sequence composed of two adjoining two EST's (nucleotides 417-633 originating from nucleotides 304-520 of NCBI accession CK272589; nucleotides 634-746 originating from the reverse complement of nucleotides 384-496 from NCBI accession BM112095) to create a frt-like site from nucleotides 618-648;
nucleotides 747-1811 are from the patatin class I promoter (nucleotides 41792-42856 of NCBI accession DQ274179);
nucleotides 1812-2588 represent the coding region of a myb transcription factor, the D locus allele Stan2⁷⁷⁷, from NCBI accession AY841129 with the addition of the first two codons of the open reading frame (Jung et al. 2009, Theoretical and Applied Genetics, 120: 45-57);
nucleotides 2589-3027 are from the patatin class I 3′ terminator sequence (nucleotides 3591-4029 of NCBI accession M18880);
nucleotides 3028-3371 represent a potato-derived DNA sequence composed of two adjoining two EST's (nucleotides 3028-3167 originating from nucleotides 381-520 of NCBI accession CK272589; nucleotides 3168-3371 originating from the reverse complement of nucleotides 293-496 from NCBI accession BM112095) to create a frt-like site from nucleotides 3157-3187;
nucleotides 3372-3374 are added to create a SmaI restriction site as a option for future cloning; and
nucleotides 3375-5628 are from the pUC57 vector.

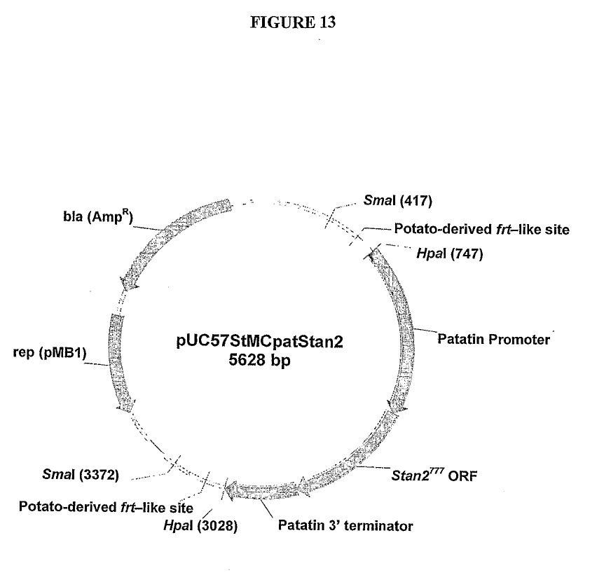

A plasmid map of 5628 bp pUC57StMCpatStan2 is illustrated in FIG. 13. The region from nucleotides 417-3371 is composed entirely of DNA sequences derived from potato and has been verified by DNA sequencing between the M13 forward and M13 reverse universal primers.

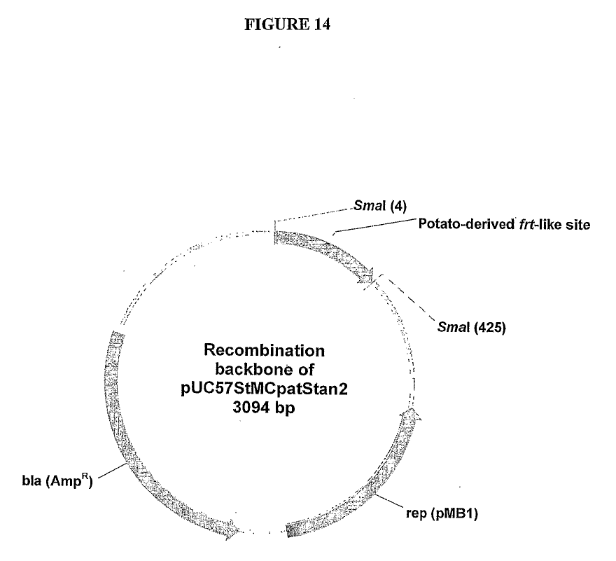

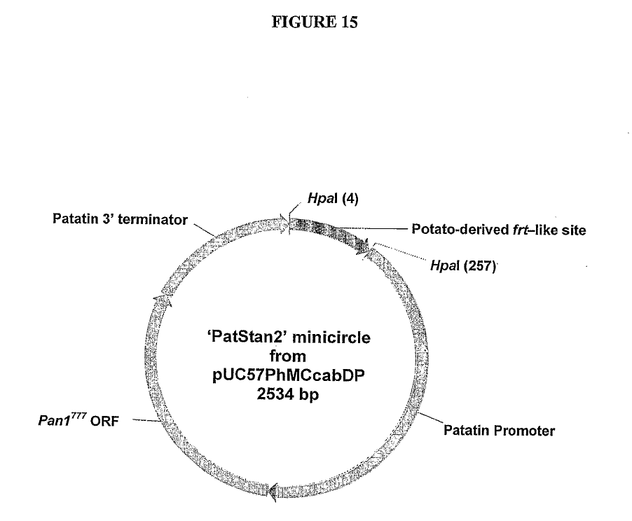

The transfer of pUC57StMCpatStan2 to Escherichia coli strain 294-FLP allows the production of potato derived minicircles by intramolecular recombination between the potato-derived frt-like sites. E. coli strain 294-FLP has FLP recombinase under the control of the heat inducible λPr promoter (Buchholz et al. 1996, Nucleic Acids Research, 24: 3118-3119). The pUC57StMCpatStan2 plasmid was maintained in E. coli strain 294-Cre by incubating at 28° C. in LB medium with 100 mg/l ampillicin. Raising the temperature to 37° C. induces the expression of FLP recombinase in E. coli strain 294-Cre, resulting in recombination between the two potato-derived frt-like sites. This produces a 3094 bp plasmid derived from the pUC57 sequence with a short region of potato DNA (FIG. 14) and the 2534 bp potato ‘patStan2’ minicircle (FIG. 15).

The 2534 bp potato ‘patStan2’ minicircle is composed entirely of DNA fragments derived from potato and contains a chimeric gene inducing the biosynthesis of anthocyanins (FIG. 15). The full sequence of the potato ‘patStan2’ minicircle is shown in SEQ ID NO:5, where:
nucleotides 1-3 are from the patatin class I 3′ terminator sequence (nucleotides 4027-4029 of NCBI accession M18880);
nucleotides 4-143 originate from nucleotides 381-520 of NCBI accession CK272589;
nucleotides 144-256 originate from the reverse complement of nucleotides 384-496 from NCBI accession BM112095;
nucleotides 128-158 represent the FLP-induced recombined potato-derived frt-like site;
nucleotides 257-1321 are from the patatin class I promoter (nucleotides 41792-42856 of NCBI accession DQ274179);
nucleotides 1322-2098 represent the coding region of a myb transcription factor, the D locus allele Stan2⁷⁷⁷, from NCBI accession AY841129 with the addition of the first two codons of the open reading frame (Jung et al. 2009, Theoretical and Applied Genetics, 120: 45-57);
nucleotides 2099-2534 are from the patatin class I 3′ terminator sequence (nucleotides 3592-4026 of NCBI accession M18880).

### (B) Potato Minicircles Based on Potato-Derived LoxP-Like Sites

A 2274 bp sequence of DNA derived from potato was assembled as an expression cassette using a combination of synthesis by Genscript Corporation (Piscatawa, N.J., www.genscript.com), followed by standard cloning by restriction and ligation. This chimeric potato gene consisted of the coding region of a potato myb transcription factor, the D locus allele Stan2⁷⁷⁷ (Jung et al. 2009, Theoretical and Applied Genetics, 120: 45-57), under the transcriptional control of the regulatory regions of the potato granule-bound starch synthase gene. This sequence, named Stan2 GBSS, is shown in SEQ ID NO:6, where:
nucleotides 1-1076 are from the promoter of the potato granule-bound starch synthase gene (nucleotides 738-1813 of NCBI accession X83220);
nucleotides 1077-1853 represent the coding region of a myb transcription factor, the D locus allele Stan2⁷⁷⁷, from NCBI accession AY841129 with the addition of the first two codons of the open reading frame (Jung et al. 2009, Theoretical and Applied Genetics, 120: 45-57); and
nucleotides 1854-2274 are from the 3′ terminator sequence of the potato granule-bound starch synthase gene (nucleotides 4801-5221 of NCBI accession X83220).

In a similar manner a 2199 bp sequence of DNA was assembled for a chimeric potato gene consisting of the coding region of a potato myb transcription factor, the D locus allele Stan2⁷⁷⁷ (Jung et al. 2009, Theoretical and Applied Genetics, 120: 45-57), under the transcriptional control of the regulatory regions of the potato patatin class I gene. This sequence, named Stan2 Patatin, is shown in SEQ ID NO:7, where:
nucleotides 1-1080 are from the potato patatin class I promoter (nucleotides 41781-42860 of NCBI accession DQ274179);
nucleotides 1081-1857 represent the coding region of a myb transcription factor, the D locus allele Stan2⁷⁷⁷, from NCBI accession AY841129 with the addition of the first two codons of the open reading frame (Jung et al. 2009, Theoretical and Applied Genetics, 120: 45-57); and
nucleotides 1858-2199 are from the potato patatin class I 3′ terminator sequence (nucleotides 3592-3933 of NCBI accession M18880.1).

The PanGBSS sequence was blunt ligated as a HindIII-DraI fragment into the unique BamHI site of pPOTLOXP2 (from Example 5) to yield pPOTLOXP2:Stan2 GBSSPT. The full sequence of pPOTLOXP2:Stan2 GBSSPT is shown in SEQ ID NO:8, where:
nucleotides 1-491 are from the vector backbone of pPOTLOXP2
nucleotides 492-1137 represent potato-derived sequences composed of two adjoining ESTs (nucleotides 492-738 originating from nucleotides 302-548 of NCBI accession BQ045786; nucleotides 739-1137 originating from nucleotides 17-415 of NCBI accession BQ111407) to create a LoxP-like sequence from nucleotides 724-757;
nucleotides 1138-1148 are from the reverse complement of nucleotides 374-384 of NCBI accession CK278818;
nucleotides 1149-2223 are from the promoter of the potato granule-bound starch synthase gene (nucleotides 739-1813 of NCBI accession X83220);
nucleotides 2224-3000 represent the coding region of a myb transcription factor, the D locus allele Stan2⁷⁷⁷, from NCBI accession AY841129 with the addition of the first two codons of the open reading frame (Jung et al. 2009, Theoretical and Applied Genetics, 120: 45-57);
nucleotides 3001-3418 are from the 3′ terminator sequence of the potato granule-bound starch synthase gene (nucleotides 4801-5218 of NCBI accession X83220);
nucleotides 3419-3600 are from the reverse complement of nucleotides 192-373, NCBI accession CK278818
nucleotides 3601-4221_represent potato-derived sequences composed of two adjoining ESTs (nucleotides 3601-3844 originating from nucleotides 305-548_of NCBI accession BQ045786;_nucleotides 3845-4221 originating from_nucleotides 17-393_of NCBI accession BQ111407) to create a LoxP-like sequence from nucleotides 3830-3863; and
nucleotides 4222-7578 are from the vector backbone of pPOTLOXP2.

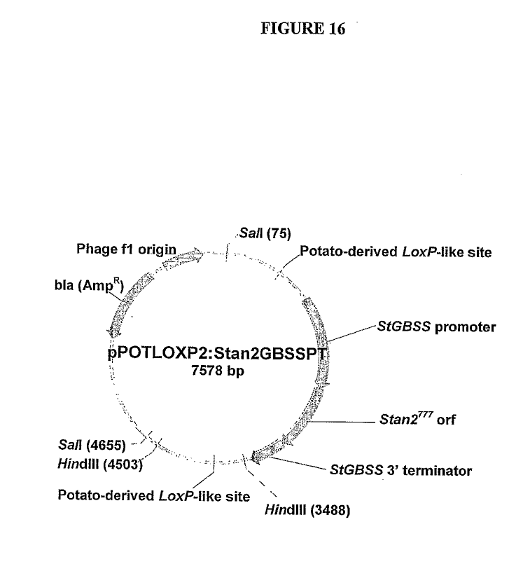

A plasmid map of the 7578 bp pPOTLOXP2:Stan2 GBSSPT is illustrated in FIG. 16. The region from nucleotides 77-4654 is composed entirely of DNA sequences derived from potato.

The Stan2 Patatin sequence was blunt ligated as a PmlI-EcoRV fragment into the unique BamHI site of pPOTLOXP2 (from Example 5) to yield pPOTLOXP2:Stan2 Patatin. The full sequence of pPOTLOXP2:Stan2 Patatin is shown in SEQ ID NO:9, where:
nucleotides 1-490 are from the vector backbone of pPOTLOXP2
nucleotides 491-1136 represent potato-derived sequences composed of two adjoining ESTs (nucleotides 491-737 originating from nucleotides 302-548 of NCBI accession BQ045786; nucleotides 738-1136 originating from nucleotides 17-415 of NCBI accession BQ111407) to create a LoxP-like sequence from nucleotides 723-756;
nucleotides 1137-1147 are from the reverse complement of nucleotides 374-384 of NCBI accession CK278818;
nucleotides 1148-2227 are from the promoter of the potato patatin class I promoter gene (nucleotides 41781-42860 of NCBI accession DQ274179);
nucleotides 2228-3004 represent the coding region of a myb transcription factor, the D locus allele Stan2⁷⁷⁷, from NCBI accession AY841129 with the addition of the first two codons of the open reading frame (Jung et al. 2009, Theoretical and Applied Genetics, 120: 45-57);
nucleotides 3005-3346 are from the 3′ terminator sequence of the potato patatin class I gene (nucleotides 3592-3933 of NCBI accession M18880.1);
nucleotides 3347-3528 are from the reverse complement of nucleotides 192-373, NCBI accession CK278818
nucleotides 3529-4149 represent potato-derived sequences composed of two adjoining ESTs (nucleotides 3529-3772 originating from nucleotides 305-548 of NCBI accession BQ045786; nucleotides 3773-4149 originating from nucleotides 17-393 of NCBI accession BQ111407) to create a LoxP-like sequence from nucleotides 3758-3791; and
nucleotides 4150-7507_are from the vector backbone of pPOTLOXP2.

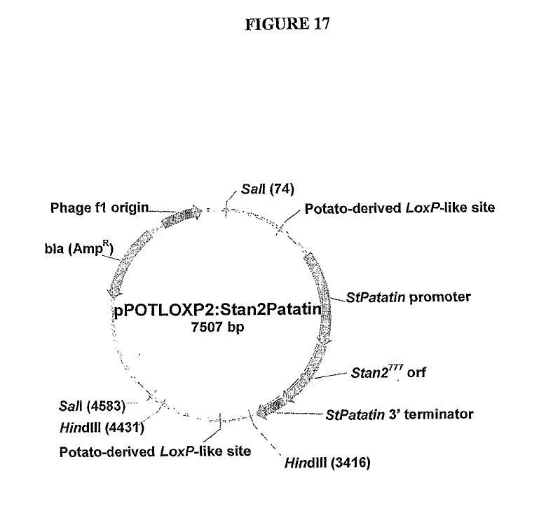

A plasmid map of the 7507 bp pPOTLOXP2:Stan2 Patatin is illustrated in FIG. 17. The region from nucleotides 76-4587 is composed entirely of DNA sequences derived from potato.

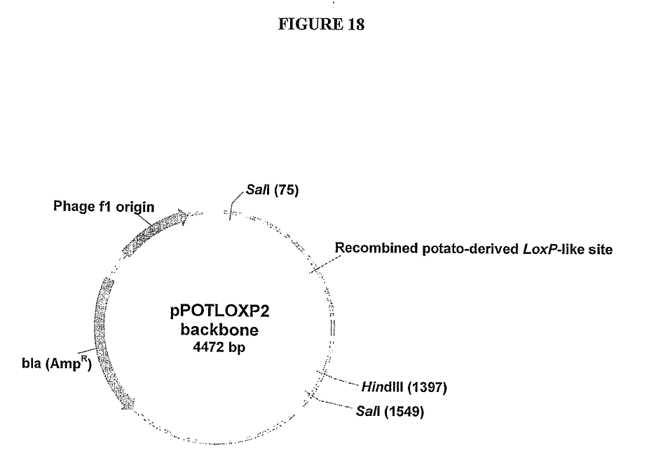

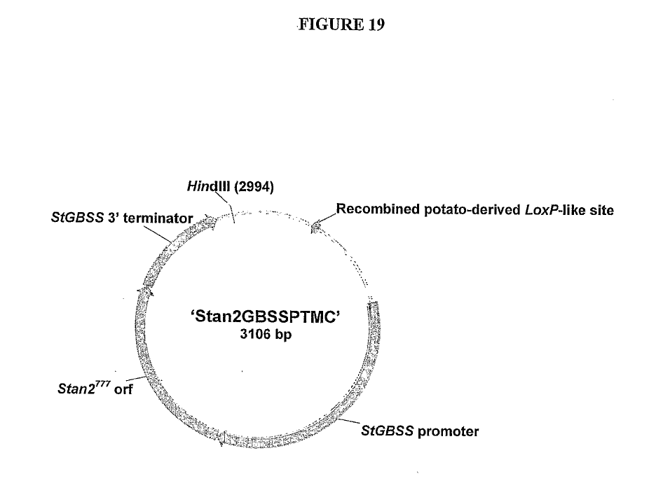

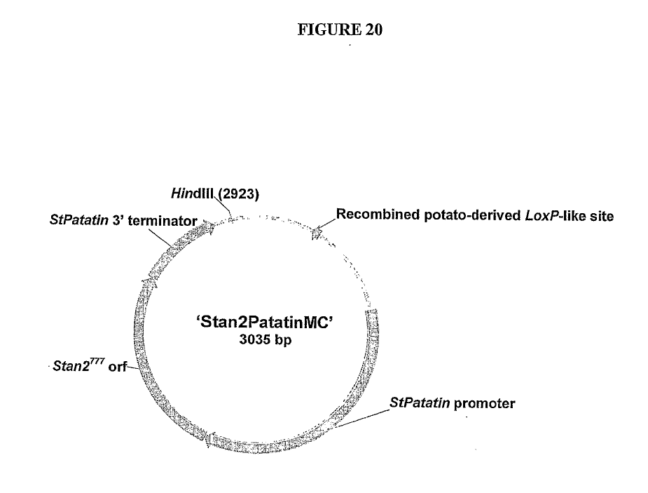

The ability for pPOTLOXP2:Stan2 GBSSPT and pPOTLOXP2:Stan2 Patatin to generate minicircles by intramolecular recombination between the potato-derived LoxP sites was tested in vivo using Escherichia coli strain 294-Cre with Cre recombinase under the control of the heat inducible λPr promoter (Buchholz et al. 1996, Nucleic Acids Research, 24: 3118-3119). The pPOTLOXP2:Stan2 GBSSPT and pPOTLOXP2:Stan2 Patatin plasmids were independently transformed into E. coli strain 294-Cre and maintained by selection in LB medium with 100 mg/l ampillicin and incubation at 28° C. Raising the temperature to 37° C. induced the expression of Cre recombinase in E. coli strain 294-Cre, resulting in recombination between the two potato-derived LoxP sites residing on each plasmid. For pPOTLOXP2:Stan2 GBSSPT this produced a 4472 bp plasmid derived from the pPOTLOXP2 sequence with a region of potato DNA (FIG. 18) and the 3106 bp potato minicircle ‘Stan2 GBSSMC’ (FIG. 19). For pPOTLOXP2:Stan2 Patatin this produced the same 4472 bp plasmid derived from the pPOTLOXP2 sequence with a region of potato DNA (FIG. 18) and the 3035 bp potato minicircle ‘Stan2 PatatinMC’ (FIG. 20).

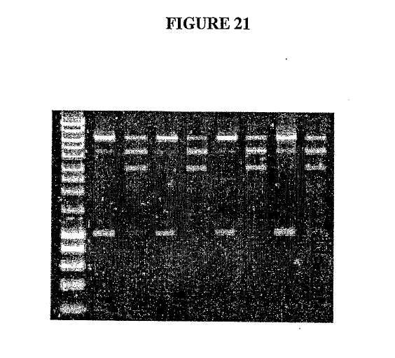

To demonstrate the production of the two potato minicircles the pPOTLOXP2:Stan2 GBSSPT and pPOTLOXP2:Stan2 Patatin plasmids were propagated in E. coli strain 294-Cre at 28° C., without and without 4 hours of Cre recombinase induction at 37° C. Plasmid preparations were then digested with HindIII. When cultured overnight at 28° C. with uninduced Cre recombinase only the expected 6563 bp and 1015 bp fragments expected for the intact pPOTLOXP2:Stan2 GBSSPT plasmid (FIG. 21, lanes 2 and 4) or the 6492 bp and 1015 bp fragments expected for the intact pPOTLOXP2:Stan2 Patatin plasmid (FIG. 21, lanes 6 and 8) were observed. After 4 hours induction at 37° C. the presence of both the 4472 bp recombination backbone plasmid and the 3106 bp potato ‘Stan2 GBSSMC’ minicircle (FIG. 21, lanes 3 and 5) or the 3035 bp potato ‘Stan2 PatatinMC’ minicircle (FIG. 21, lanes 7 and 9) were evident.

To produce larger quantities of the potato minicircles for plant transformation several 50 ml cultures of E. coli strain 294-Cre with pPOTLOXP2:Stan2 GBSSPT or pPOTLOXP2:Stan2 Patatin were cultured overnight on a shaker at 28° C. in liquid LB medium with 100 mg/l ampillicin. After overnight growth, the cultures were transferred to 37° C. to induce Cre expression and recombination. After 4 hours at 37° C., the cultures were centrifuged at 4,000 rpm for 20 minutes and the well-drained pellets of E. coli cells were stored at −20° C. and subsequently DNA purification was carried out by alkaline lysis and ethanol precipitation (Sambrook et al. 1987, Molecular Cloning: A Laboratory Manual, 2^{nd} ed., Cold Spring Harbor Press). The DNA pellets were completely dried, then dissolved in 500 μl TE (pH 8.0) plus 100 μg/ml RNase A.

The DNA was then restricted overnight at 37° C. with SalI to linearise the 4472 bp pPOTLOXP2-based backbone plasmid (see FIG. 18) and any remaining pPOTLOXP2:Stan2 GBSSPT plasmid (see FIG. 16) or pPOTLOXP2:Stan2 Patatin plasmid (see FIG. 17), but leaving the 3106 bp circular potato ‘Stan2 GBSSMC’ minicircle (see FIG. 16) or the 3035 bp circular potato ‘Stan2 PatatinMC’ minicircle (see FIG. 20) intact. Following restriction, DNA was passed through Qiagen PCR purification columns and eluted with 50 μl of distilled H₂O. The purified digests were then treated with λ Exonuclease (NEB M0262S) following the manufacturer's guidelines and incubated at 37° C. for 4 hours to digest the linear DNA. The exonuclease was then heat inactivated at 72° C. for 10 minutes. The samples were purified by passing through Qiagen PCR purification columns and eluted with 50 μl of distilled H₂O to yield the remaining intact 3106 bp circular potato ‘Stan2 GBSSMC’ minicircle (see FIG. 19) or the 3035 bp circular potato ‘Stan2 PatatinMC’ minicircle (see FIG. 20) intact.

The purified ‘Stan2 GBSSMC’ minicircle is composed entirely of DNA fragments derived from potato and contains a chimeric gene for induction of the biosynthesis of anthocyanins. The full sequence of the ‘Stan2 GBSSMC’ minicircle is shown in SEQ ID NO:10, where:
nucleotides 1-244 are nucleotides 305-548 of NCBI accession BQ045786;
nucleotides 245-643 are nucleotides 17-415 of NCBI accession BQ111407;
nucleotides 320-263 represent the Cre-induced recombined potato-derived LoxP-like site;
nucleotides 644-654 are from the reverse complement of nucleotides 374-384 of NCBI accession CK278818;
nucleotides 655-1729 are from the promoter of the potato granule-bound starch synthase gene (nucleotides 739-1813 of NCBI accession X83220);
nucleotides 1730-2506 represent the coding region of a myb transcription factor, the D locus allele Stan2⁷⁷⁷, from NCBI accession AY841129 with the addition of the first two codons of the open reading frame (Jung et al. 2009, Theoretical and Applied Genetics, 120: 45-57);
nucleotides 2507-2924 are from the 3′ terminator sequence of the potato granule-bound starch synthase gene (nucleotides 4801-5218 of NCBI accession X83220); and
nucleotides 2925-3106 are from the reverse complement of nucleotides 192-373 of NCBI accession CK278818.

The purified ‘Stan2 PatatinMC’ minicircle is composed entirely of DNA fragments derived from potato and contains a chimeric gene for induction of the biosynthesis of anthocyanins. The full sequence of the ‘Stan2 PatatinMC’ minicircle is shown in SEQ ID NO 11, where:
nucleotides 1-244 are nucleotides 305-548 of NCBI accession BQ045786;
nucleotides 245-643 are nucleotides 17-415 of NCBI accession BQ111407;
nucleotides 320-263 represent the Cre-induced recombined potato-derived LoxP-like site;
nucleotides 644-654 are from the reverse complement of nucleotides 374-384 of NCBI accession CK278818;
nucleotides 655-1734 are from the promoter of the potato patatin class I promoter gene (nucleotides 41781-42860 of NCBI accession DQ274179);
nucleotides 1735-2511 represent the coding region of a myb transcription factor, the D locus allele Stan2⁷⁷⁷, from NCBI accession AY841129 with the addition of the first two codons of the open reading frame (Jung et al. 2009, Theoretical and Applied Genetics, 120: 45-57);
nucleotides 2512-2853 are from the 3′ terminator sequence of the potato patatin class I gene (nucleotides 3592-3933 of NCBI accession M18880.1); and
nucleotides 2854-3035 are from the reverse complement of nucleotides 192-373 of NCBI accession CK278818.

Potato (Solanum tuberosum L.) plants were transformed with the 3106 bp ‘Stan2 GBSSMC’ minicircle DNA using standard biolistic approaches. Young greenhouse grown potato leaves from the cultivar Purple Passion were harvested and surface-sterilised by immersion with gentle shaking for 10 minutes in 10% commercial bleach (1.5% sodium hypochlorite) containing a few drops of 1% Tween 20, followed by several washes with sterile distilled water. A biolistic gold preparation was then made using a standard protocol: 1 μg of minicircle DNA, 20 μl of 0.1 M spermidine and 50 μl of 2.5 M CaCl₂ were mixed with a suspension containing 50 mg of sterile 1.0 μm diameter gold particles to give a total volume of 130 μl. After 5 minutes 95 μl of supernatant was discarded leaving 35 μl of DNA-bound gold suspension.

The leaf pieces were then bombarded using a particle in-flow gun. Each leaf piece was bombarded twice with 5 μl of the gold suspension. The leaf pieces were then cut into small sections (approximately 5 mm²) and transferred to potato regeneration media consisting of MS salts and vitamins (Murashige & Skoog 1962, Physiologia Plantarum, 15: 473-497), 5 g/l sucrose, 40 mg/l ascorbic acid, 500 mg/l casein hydrolysate, plus 1.0 mg/l zeatin and 5 mg/l GA₃ (both filter sterilised and added after autoclaving) and 7 g/l agar at pH5.8. These were cultured at 25° C. under cool white fluorescent lamps (70-90 μmol/m²/s; 16-h photoperiod). After 15 days RNA was isolated from of tissue from the shot zone. Leaf tissue was frozen in liquid nitrogen and ground to a powder. For 1 g of leaf tissue, one volume of GNTC (4M guanidine thiocyanate, 25 mM sodium citrate, 0.5% sodium lauryl sarcosinate, pH 7.0, with 8 μl/ml 2-mercaptoethanol added just prior to use), 0.1 volume 2M NaOAc at pH4, and one volume of phenol were added and thoroughly mixed by vortexing. Then 0.3 volume of chloroform:isoamyl alcohol (49:1) was added and thoroughly mixed by vortexing again, followed by centrifugation at 12000 rpm for 15 min at 4° C. The aqueous phase (500 μl) was collected and the RNA was precipitated with one volume cold isopropanol. After centrifugation at 14000 rpm for 15 min at 4° C., the supernatant was decanted off and pellet washed with 300 μl 70% ethanol. The pellet was dissolved in 30 μl sterile water.

RT-PCR was performed using the primers Panfrt For (^{5′}TGCAATGAAATTGATAAAACACC^{3′}—SEQ ID NO: 62) and GBSSTermRev (^{5′}TCATCAAAGGAGGACGGAGCAAGA^{3′}—SEQ ID NO: 63) which produce an expected product of 494 bp bridging the Stan2⁷⁷⁷ coding region and the 3′ terminator sequence of the potato granule-bound starch synthase gene. This transcription product is from a chimeric potato gene it is only expected from tissue transformed with the ‘Stan2 GBSSMC’ minicircle and not from wild-type potato. First strand cDNA was synthesised using SuperScript™ II Reverse Transcriptase (Invitrogen, Carlsbad, Calif.) according to manufacturer's instruction. RT-PCR was carried out in a DNA engine Thermal Cycler (Bio-Rad, California, USA). The reaction included 1 μl Taq DNA polymerase (5U/μl; Roche, Mannheim, Germany), 41 10×PCR reaction buffer with MgCl₂ (Roche), 0.5 μl of dNTP mix (10 mM of each dNTP), 0.5 μl of each primer (at 10 μM), 5 μl of cDNA or RNA (50-100 ng) and water to total volume of 20 μl. The conditions for RT-PCR were: 2 min at 94° C. (to denature the SuperScript™ II RT enzyme), 35 cycles of 30 s 94° C., 30 s 57° C., 30 s 72° C. (PCR amplification), followed by 2 min extension at 72° C., then holding the reaction at 14° C. Amplified products were separated by electrophoresis in a 2% agarose gel and visualized under UV light after staining with ethidium bromide. Two PCR negative controls were used: RNA isolated from the shot zone (from which the cDNA was made) and cDNA from wild type potato leaves shot with only gold particles. The cDNA from the shot zone yielded a band of the predicted 494 bp size. No such band was observed in either of the two negative controls, showing that the positive result was from the cDNA sample and not from non-integrated DNA from the shot event or from an endogenous gene product.

### Example 3

### Design, Construction, Production and Use of Transgenic T-DNA Minicircles for Agrobacterium-Mediated Gene Transfer

T-DNA constructs were designed to generate T-DNA minicircles in bacteria from which gene transfer to plants can be achieved by Agrobacterium-mediated transformation. In this manner the T-strand formation during Agrobacterium-mediated gene transfer can be limited to the DNA on the minicircle, thereby eliminating the opportunity for vector backbone sequences to be transferred to plants.

(A) T-DNA Region with an Intact Kanamycin Resistance, Marker Gene Capable of Forming a Minicircle.

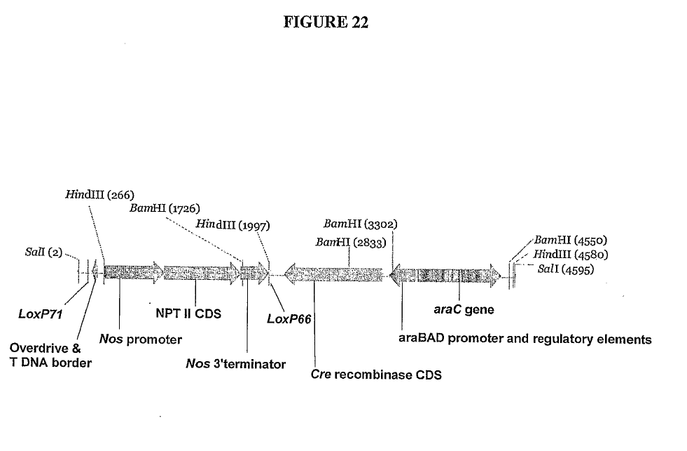

A designed vector insert is illustrated in FIG. 22. It consists of a T-DNA region for Agrobacterium-mediated gene transfer consisting of a T-DNA border and overdrive sequences, the nopaline synthase promoter (pNOS), the NPTII coding region and the nopaline synthase 3′ terminator. The T-DNA region is bound by LoxP sites at each end. The vector insert also contains the Cre gene for the site specific recombinase under the expression control of the araBAD promoter (PBAD). Induction of Cre recombinase effects site specific recombination between the two LoxP sites, thereby generating a small T-DNA minicircle.

Expression of PBAD is both positively and negatively regulated by the product of the araC gene (Ogden et al. 1980, Proceedings of the National Academy of Sciences USA 77: 3346-3350), a transcriptional regulator that forms a complex with L-arabinose. When arabinose is not present, a dimer of AraC dimer forms a 210 bp DNA loop by bridging the O₂ and I₁ sites of the araBAD operon. Maximum transcriptional activation occurs when arabinose binds to AraC. This releases the protein from the O₂ site, which now binds the I₂ site adjacent to the I₁ site. This liberates the DNA loop and allows transcription to begin (Soisson et al. 1997, Science 276: 421-425). The binding of AraC to I1 and I2 is facilitated by the cAMP activator protein (CAP)-cAMP complex binding to the DNA. Repression of basal expression levels can be enhanced by introducing glucose to the growth medium. Glucose acts by lowering cAMP levels, which in turn decreases the binding of CAP. As cAMP levels are lowered, transcriptional activation is decreased, which is necessary when expression of the protein of interest is undesirable (Hirsh et al. 1977, Cell 11: 545-550).

The first step toward the construction of the vector insert illustrated in FIG. 22 involved the design of the minicircle forming T-DNA region. The 248 bp sequence shown in SEQ ID NO: 12 was assembled in silico, where:
nucleotides 2-7 represent the XbaI restriction enzyme recognition site;
nucleotides 8-15 represent the NotI restriction enzyme recognition site;
nucleotides 16-49 represent the LoxP site loxP66;
nucleotides 50-55 represent the BglII restriction enzyme recognition site;
nucleotides 56-61 represent the PstI restriction enzyme recognition site;
nucleotides 62-67 represent the HindIII restriction enzyme recognition site;
nucleotides 68-73 represent the AatII restriction enzyme recognition site;
nucleotides 74-79 represent the Acc65I/KpnI restriction enzyme recognition site;
nucleotides 80-85 represent the SpeI restriction enzyme recognition site;
nucleotides 86-91 represent the Bsp1407I/BsrG1 restriction enzyme recognition site;
nucleotides 92-97 represent the SmaI/XmaI restriction enzyme recognition site;
nucleotides 98-103 represent the EcoRI restriction enzyme recognition site;
nucleotides 104-109 represent the AccIII/BspE1 restriction enzyme recognition site;
nucleotides 110-115 represent the MfeI/MunI restriction enzyme recognition site;
nucleotides 116-121 represent the SplI/BsiWI restriction enzyme recognition site;
nucleotides 122-127 represent the SacI/SstI restriction enzyme recognition site;
nucleotides 128-133 represent the XhoI restriction enzyme recognition site;
nucleotides 134-139 represent the AvrII restriction enzyme recognition site;
nucleotides 140-164 represent a T-DNA border sequence from Agrobacterium;
nucleotides 165-188 represent the overdrive sequence from Ti plasmid of Agrobacterium (octopine strains);
nucleotides 189-194 represent the ClaI/BspDI restriction enzyme recognition site;
nucleotides 195-200 represent the ApaI restriction enzyme recognition site;
nucleotides 201-234 represent the LoxP site loxP71 ;
nucleotides 235-242 represent the NotI restriction enzyme recognition site;
nucleotides 243-248 represent the SalI restriction enzyme recognition site.

This sequence was synthesised by Genscript Corporation (Piscatawa, N.J., USA, www.genscript.com) and cloned into pUC57 to give pUC57LoxP. The inserted sequence has been verified by DNA sequencing between the M13 forward and M13 reverse universal primers. All subsequent plasmid constructions were performed using standard molecular biology techniques of plasmid isolation, restriction, ligation and transformation into Escherichia coli strain DH5α (Sambrook et al. 1987, Molecular Cloning: A Laboratory Manual, 2^{nd} ed., Cold Spring Harbor Press). In some instances DNA preparations were performed in Escherichia coli strain SCS110 when cleavage with methylation sensitive restriction enzymes was required.

The 227 bp NotI fragment from pUC57LoxP was cloned into pART7 (Gleave 1992, Plant Molecular Biology, 20: 1203-1207) to replace the resident Nod fragment comprising the 35S-mcs-osc cassette, resulting in p7LoxP. The NPTII coding region flanked by the nopaline synthase promoter and 3′ terminator region was then excised as a 1731 bp HindIII fragment from pMOA33 (Barrell and Conner 2006, BioTechniques, 41: 708-710) and ligated between LoxP66 and the T-DNA border/overdrive of p7LoxP to give p7LoxPKan.

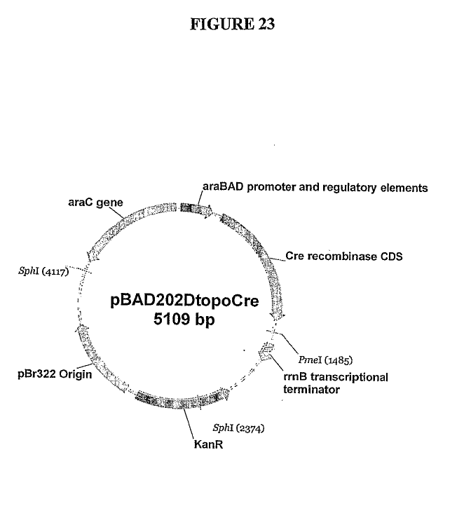

The second step toward the construction of the vector insert illustrated in FIG. 22 involved the assembly of the arabinose-inducible Cre recombinase cassette. Using DNA from pUC57LacICre (Plant & Food Research) and the primers CreFor (^{5′}CCACATGTCCAATTTACTGACCGTTACAC^{3′}—SEQ ID NO: 13) and Cre Rev (^{5′}GTCGACGCGGCCGCTCTA^{3′}—SEQ ID NO: 14), a polymerase chain reaction was performed using high fidelity Vent polymerase (NEB, Beverly, Mass., USA) to amplify the Cre recombinase gene. The resulting 1056 bp PCR product and the 4053 bp HindIII-NcoI fragment of pBAD202Dtop( ) (Invitrogen, Carlsbad, Calif.) were blunt ligated following treatment of the two fragments with Quick Blunting Kit (NEB, Beverly, Mass., USA). In the resulting plasmid, pBAD202DtopoCre (FIG. 23), the araBAD-Cre cassette, including the araC gene, is located on a 2477 bp SphI-PmeI fragment.

The minicircle forming T-DNA region and the arabinose-inducible Cre recombinase cassette were cloned onto the vector backbone of pART27 (Gleave 1992, Plant Molecular Biology, 20: 1203-1207) for maintenance in Agrobacterium. To generate appropriate cloning sites on pART27, the T-DNA bound by SalI restriction enzyme recognition sites was first replaced with the multiple cloning site from pBLUESCRIPT. The 224 bp product of a polymerase chain reaction using pBLUESCRIPT DNA and the universal M13 forward and M13 reverse primers was blunt ligated to the 8008 bp Sail vector backbone of pART27, following treatment of the two fragments with the Quick Blunting Kit (NEB, Beverly, Mass., USA). The resulting 8235 bp plasmid was designated pART27MCS.

The 1958 bp NotI fragment from p7LoxPKan comprising the minicircle forming T-DNA region was cloned into the NotI site of pART27MCS. The resulting plasmid was restricted with XbaI and blunt ligated with the 2477 bp SphI-PmeI fragment of pBAD202DtopoCre following the treatment of both fragments with the Quick Blunting Kit (NEB, Beverly, Mass., USA). The completed plasmid was designated pMOA38. The full sequence of the region cloned onto the 8235 bp backbone of pART27MCS is shown in SEQ ID NO: 15, where:
nucleotides 1-6 represent the SalI restriction enzyme recognition site from pART27MCS;
nucleotides 7-97 represent vector sequence from pART27MCS consisting of restriction enzyme recognition sites for Sad (nucleotides 74-79) and NotI (nucleotides 90-97);
nucleotides 98-131 represent the LoxP site loxP71;
nucleotides 132-137 represent the ApaI restriction enzyme recognition site;
nucleotides 138-143 represent the ClaI restriction enzyme recognition site;
nucleotides 144-192 represent the overdrive sequence from Ti plasmid of Agrobacterium (octopine strains) and a T-DNA border sequence from Agrobacterium;
nucleotides 193-264 represent a multiple cloning site from pUC57LoxP consisting of restriction enzyme recognition sites for AvrII, XhoI, Sad, SplI, MfeI, AccIII, EcoRI, SmaI/XmaI, Bsp1407I, SpeI, Acc65I/KpnI and AatII;
nucleotides 265-270 represent the HindIII restriction enzyme recognition site;
nucleotides 266-2000 represent the nopaline synthase promoter (nucleotides 266-897); the neomycin phosphotransferase II (NPTII) coding region (nucleotides 898-1701) and the nopaline synthase 3′ terminator region (nucleotides 1702-2000) on a 1731 bp HindIII fragment;
nucleotides 1996-2001 represent the HindIII restriction enzyme recognition site;
nucleotides 2002-2007 represent the PstI restriction enzyme recognition site;
nucleotides 2008-2013 represent the BglII restriction enzyme recognition site;
nucleotides 2014-2047 represent the LoxP site loxP66;
nucleotides 2048-2055 represent the NotI restriction enzyme recognition site;
nucleotides 2056-2060 represent the blunted XbaI restriction enzyme recognition site;
nucleotides 2061-4537 represent the arabinose-inducible Cre recombinase under control of the araBAD promoter on a blunted 2477 bp SphI-PmeI fragment, consisting of the Cre recombinase coding region (nucleotides 2161-3192), araBAD promoter and regulatory elements (nucleotides 3269-3514) and the araC gene (nucleotides 3571-4449);
nucleotides 4538-4542 represent the blunted XbaI restriction enzyme recognition site;
nucleotides 4543-4621 represent vector sequence from pART27MCS consisting of restriction enzyme recognition sites for SpeI, BamHI, SmaI/XmaI, PstI, EcoRI, EcoRV, HindIII, ClaI, SalI, XhoI, ApaI and KpnI; and
nucleotides 4622-12674 represent vector backbone of pART27MCS.

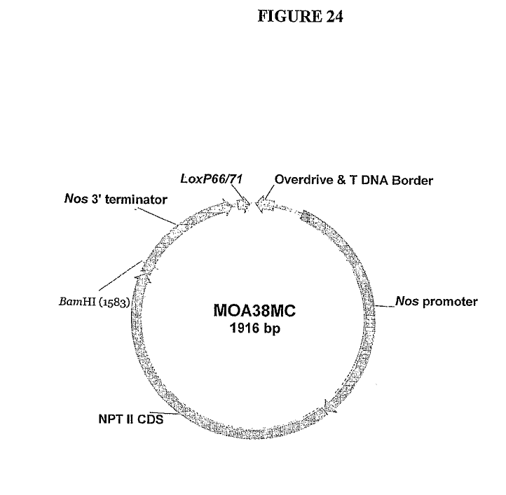

When the binary vector pMOA38 is propagated in Escherichia coli or Agrobacterium, the presence of arabinose induces the expression of Cre recombinase which results in intramolecular recombination between the LoxP66 and LoxP71 sites and produces a T-DNA minicircle and a residual plasmid of the remaining sequences. The T-DNA minicircle is illustrated in FIG. 24 and defines a minimal unit from which a well defined T-strand can be synthesised, without vector backbone sequences, during Agrobacterium-mediated gene transfer. The full sequence of this minicircle, MOA38MC, is shown in SEQ ID NO: 16, where:
nucleotides 1-24 represent the overdrive sequence from Ti plasmid of Agrobacterium (octopine strains);
nucleotides 25-49 represent a T-DNA border sequence from Agrobacterium with T-strand expected to initiate about nucleotide 47 (see arrow);
nucleotides 50-121 represent a multiple cloning site from pUC57LoxP consisting of restriction enzyme recognition sites for AvrII, XhoI, SacI, SplI, MfeI, AccIII, EcoRI, SmaI/XmaI, Bsp1407I, SpeI, Acc65I/KpnI and AatII.
nucleotides 122-127 represent the HindIII restriction enzyme recognition site
nucleotides 127-1857 represent the nopaline synthase promoter (nucleotides 127-754); the neomycin phosphotransferase II (NPTII) coding region (nucleotides 755-1558) and the nopaline synthase 3′ terminator region (nucleotides 1559-1857) on a 1731 bp HindIII fragment;
nucleotides 1853-1858 represent the HindIII restriction enzyme recognition site
nucleotides 1859-1864 represent the PstI restriction enzyme recognition site
nucleotides 1865-1870 represent the BglII restriction enzyme recognition site
nucleotides 1871-1904 represent a recombined LoxP site with nucleotides 1871-1887 originating from loxP66 and nucleotides 1888-1904 originating from loxP71;
nucleotides 1905-1910 represent the ApaI restriction enzyme recognition site
nucleotides 1911-1916 represent the ClaI restriction enzyme recognition site

Following arabinose induction of the minicircle from pMOA38, the presence of minicircles can be conveniently verified by restricting plasmid preparations with BamHI. The 12,674 bp parent plasmid pMOA38 gives rise to fragments of 9850, 1248, 1107, and 469 bp. The T-DNA minicircle produces a 1916 bp fragment and the recombined plasmid backbone results in 9041, 1248, and 469 bp fragments. As expected, overnight cultures of Escherichia coli DH5α with pMOA38 in LB plus 100 μg/ml spectinomycin and 0.2% glucose failed to produce minicircles. From this overnight culture, 10 μl was transferred to fresh LB medium with 100 μg/ml spectinomycin, grown for 2 hours at 37° C. and 1000 rpm until OD₆₀₀=0.5, then grown in the same medium, or with the addition of 0.2% glucose, 0.002% L-arabinose, 0.02% L-arabinose, 0.2% L-arabinose, 2% L-arabinose or 20% L-arabinose for 4 hours. Minicircles were only observed following 4 hour induction with 20% L-arabinose and 2% L-arabinose, with a trace presence of minicircles following 4 hour induction with 0.2% L-arabinose. No minicircle induction was observed, even in the absence of glucose or less than 0.2% L-arabinose.

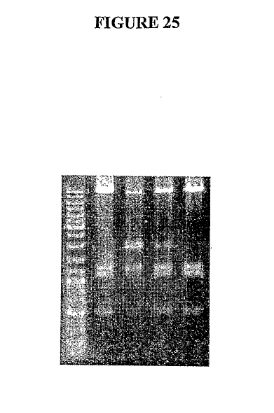

The experiment to confirm the production of minicircles was repeated in overnight cultures of Escherichia coli DH5α with pMOA38. Cultures were incubated in LB plus 100 μg/ml spectinomycin at 1000 rpm overnight at 37° C. with the addition of 0.2%, 2% or 20% L-arabinose or 0.2%, 2% or 20% D-arabinose. Following the restriction of plasmid preparations with BamHI, the induction of minicircles was only evident in the presence of L-arabinose, with very high yields in response to induction 20% L-arabinose (FIG. 25). Most importantly, the presence of the minicircle was stable in overnight cultures and highly recoverable.

The pMOA38 binary vector was transformed into the disarmed Agrobacterium tumefaciens strain EHA105 (Hood et al 1993, Transgenic Research, 2: 208-218), using the freeze-thaw method (Hagen and Willmitzer 1988, Nucleic Acids Research, 16: 9877). The Agrobacterium culture was cultured overnight at 28° C. in LB broth supplemented with 300 μg/ml spectinomycin and 200 mM L-arabinose and used to transform tobacco (Nicotiana tabacum ‘Petit Havana SR1’), essentially as previously described (Horsch et al. 1985, Science, 227: 1229-1231).

Seed was sown in vitro on a medium consisting of MS salts and vitamins (Murashige and Skoog 1962, Physiologia Plantarum, 15: 473-497) plus 30 g/l sucrose and 8 g/l agar, with pH was adjusted to 5.8 with 0.1 M KOH prior to the addition of the agar. Plants were used for transformation when leaves were about 2-3 cm wide. Leaves from the in vitro plants were excised, cut in across the midribs in strips of 5-8 mm, and submerged in the liquid Agrobacterium culture. After about 30 sec, these leaf segments were then blotted dry on sterile filter paper (Whatman® No. 1, 100 mm diameter). They were then cultured on a medium consisting of MS salts and vitamins (Murashige and Skoog 1962, Physiologia Plantarum, 15: 473-497) plus 30 g/l sucrose, 1 mg/l benzylaminopurine and 8 g/l agar in standard plastic Petri dishes (9 cm diameter×1 cm high). After two days, the leaf segments were transferred to the same medium supplemented with 200 mg/l Timentin™ to prevent Agrobacterium overgrowth and 100 mg/l kanamycin to select for transformed tobacco shoots. Regenerated shoots were transferred to MS salts and vitamins (Murashige and Skoog 1962, Physiologia Plantarum, 15: 473-497) plus 30 g/l sucrose, 100 mg/l Timentin™, 50 mg l⁻¹ kanamycin and 8 g/l agar. Following root formation the resulting putatively transformed plants were transferred to the greenhouse. All media were autoclaved at 121° C. for 15 minutes and dispensed into pre-sterilised plastic containers (80 mm diameter×50 mm high; Vertex Plastics, Hamilton, New Zealand). All antibiotics were filter sterilised and added, as required, just prior to dispensing the media into the culture vessels. Cultures were incubated at 26° C. under cool white fluorescent lamps (80-100 μmol m⁻²s⁻¹; 16-h photoperiod).

Genomic DNA was isolated from in vitro shoots of putative transgenic and control plants based on a previously the described method (Bematzky and Tanksley 1986, Theoretical and Applied Genetics, 72: 314-339). DNA was amplified in a polymerase chain reaction (PCR) containing primers specific for the either the T-DNA minicircle (across the recombined LoxP sites) or the unrecombined T-DNA in the parent binary vector pMOA38. The primer pairs used were:
(i) LOXPMCF2 (^{5′}GGTTGGGAAGCCCTGCAAAGTAAA^{3′}—SEQ ID NO: 17) and LOXPMCR2 (^{5′}TCGCTGTATGTGTTTGTTTGAT^{3′}—SEQ ID NO: 18) producing an expected product of 1561 bp from the minicircle T-DNA, but no product from the parent plasmid pMOA38 since the primers are orientated in opposite directions; and
(ii) CreFor New (^{5′}TCTTGCGAACCTCATCACTCGTTG^{3′}—SEQ ID NO: 19) and CreRevNew (^{5′}CTAATCCCTAACTGCTGGCGGAAA^{3′}—SEQ ID NO: 20) producing an expected product of 1119 bp from the parent plasmid pMOA38 but not from the minicircle T-DNA since the sequence is not present.

PCRs were carried out in a Mastercycler (Eppendorf, Hamburg, Germany). The reactions included 10 μl 5× Phusion™ HF Buffer (with 7.5 mM MgCl₂, which provides 1.5 mM MgCl₂ in final reaction conditions), 1 μl dNTP (at 10 mM each of dATP, dCTP, dGTP, dTTP), 0.5 μl Phusion™ High-Fidelity DNA Polymerase at 2 U μ/l (Finnzymes Oy, Espoo, Finland), 0.1 μl of each primer (at 100 μM), 1.0 μl of DNA (10-50 ng) and water to a total volume of 50 μl. The conditions for PCR were: 30 s at 98° C., followed by 30 cycles of 10 s 98° C., 30 s 58° C., 45 s 72° C., followed by a 10 min extension at 72° C. Amplified products were separated by electrophoresis in a 1% agarose gel and visualized under UV light after staining with ethidium bromide.

Nine independently regenerated kanamycin-resistant tobacco plants were confirmed as being PCR-positive for the expected 1561 bp product when using LOXPMCF2/LOXPMCR2 primer pairs (JNT02-3, JNT02-8, JNT02-9, JNT02-18, JNT02-22, JNT02-28, JNT02-55, JNT02-56, and JNT02-60). Three of these plants were also PCR-positive for the expected 1119 bp product from the CreFor New and CreRevNew primer pair, establishing that they were also co-transformed with the T-DNA from the parent pMOA38 binary vector also containing the functional NPTII gene (JNT02-3, JNT02-8 and JNT02-55). Six of the plants were PCR-positive for only the expected products of the LOXPMCF2/LOXPMCR2 primer pairs (JNT02-9, JNT02-18, JNT02-22, JNT02-28, JNT02-56, and JNT02-60). These plants were therefore derived from only the minicircle T-DNA.

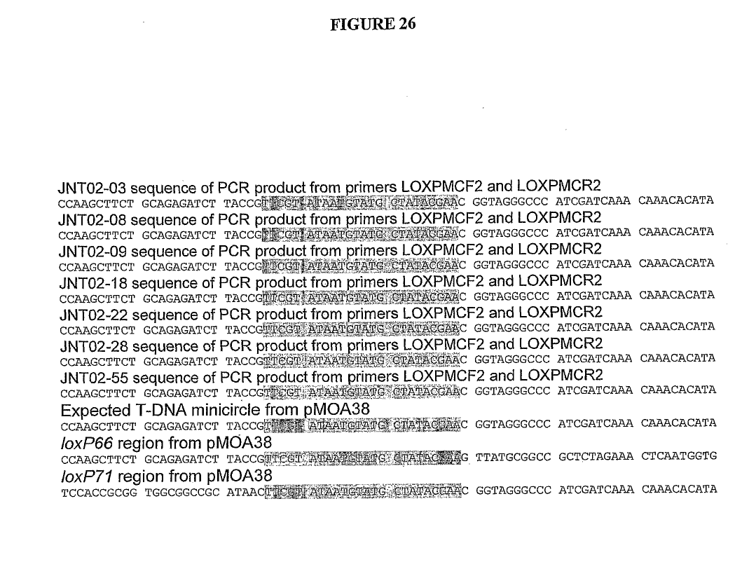

The PCR using the LOXPMCF2/LOXPMCR2 primers pairs generated a product across the intramolecular recombination event between the loxP66 and loxP71 sites. These PCR products were therefore sequenced to verify their authenticity and the fidelity of the arabinose-inducible Cre recombinase event to produce the T-DNA minicircle (FIG. 26). The DNA sequence from transformed tobacco plants (JNT02-3, JNT02-8, JNT02-9, JNT02-18, JNT02-22, JNT02-28 and JNT02-55) and the expected minicircle from pMOA38 are all identical to one another. These sequences are identical to the first part of the sequence from the loxP66 region of pMOA38 and the latter part of the sequence from the loxP71 region from pMOA38. This confirmed that the desired recombination events were induced in Agrobacterium prior to tobacco transformation and were base pair faithful when the minicircles formed.

Three transformed plants derived from only the minicircle T-DNA (JNT02-18, JNT02-56, and JNT02-60) were self-pollinated and backcrossed as a pollen and ovule parent to the non-transformed wild-type ‘Petit Havana SR1’ tobacco. The progeny were screened for kanamycin resistance as previously described (Conner et al. 1998, Molecular Breeding, 4: 47-58). The segregation of kanamycin resistance in the self-pollinated progeny of these plants did not deviate from an expected 3:1 ratio as determined by ‘Goodness of Fit’ Chi-square tests for all independent pollination events (Table 1). Likewise, in all backcrosses the segregation did not deviate from an expected 1:1 ratio as determined by ‘Goodness of Fit’ Chi-square tests. These results establish that the progeny segregated for kanamycin resistance and kanamycin sensitivity in ratios expected for a single locus insertion of the NPTII gene from the T-DNA minicircle.

TABLE 1
The inheritance of kanamycin resistance in tobacco (Nicotiana tabacum
‘Petit Havana SR1’) following Agrobacterium-mediated
transformation using T-DNA minicircles from pMOA38.

| | | Number of | Number of | | |
|---|---|---|---|---|---|
| | | kanamycin- | kanamycin- | | |
| | | resistant | susceptible | | |
| Plant line | Cross | progeny | progeny | Ratio | Chi-square |
| Wild-type | Selfed | 0 | 227 | 0:1 | — |
| | Selfed | 0 | 313 | 0:1 | — |
| JNT2-18 | Selfed | 94 | 37 | 3:1 | 0.65 |
| | Selfed | 91 | 28 | 3:1 | 0.18 |
| | Selfed | 96 | 30 | 3:1 | 0.10 |
| | 2-18 × wt | 61 | 52 | 1:1 | 0.72 |
| | 2-18 × wt | 56 | 45 | 1:1 | 1.20 |
| | 2-18 × wt | 108 | 108 | 1:1 | 0.00 |
| | wt × 2-18 | 32 | 26 | 1:1 | 0.62 |
| | wt × 2-18 | 41 | 40 | 1:1 | 0.01 |
| JNT2-56 | Selfed | 101 | 32 | 3:1 | 0.04 |
| | Selfed | 119 | 39 | 3:1 | 0.01 |
| | Selfed | 86 | 20 | 3:1 | 2.13 |
| | 2-56 × wt | 71 | 93 | 1:1 | 2.95 |
| | 2-56 × wt | 89 | 87 | 1:1 | 0.01 |
| | 2-56 × wt | 54 | 60 | 1:1 | 0.32 |
| | wt × 2-56 | 61 | 62 | 1:1 | 0.01 |
| JNT2-60 | Selfed | 82 | 29 | 3:1 | 0.05 |
| | Selfed | 54 | 16 | 3:1 | 0.17 |
| | 2-60 × wt | 90 | 76 | 1:1 | 1.18 |
| | 2-60 × wt | 110 | 102 | 1:1 | 0.30 |

(B) T-DNA Region with a Non-Functional Kanamycin Resistance Marker Gene that has Restored Function Only after Minicircle Formation.

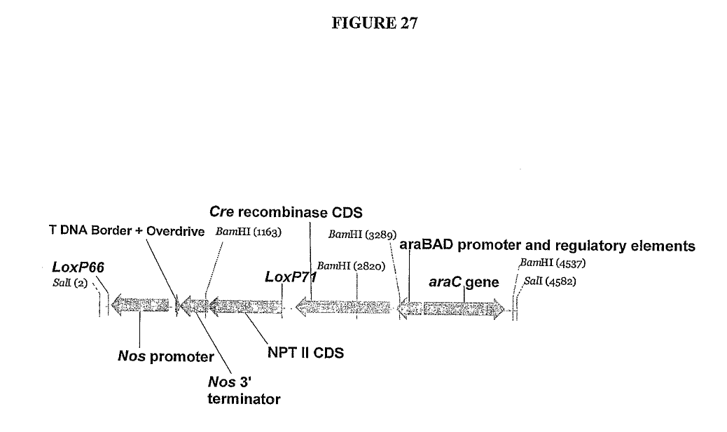

Another designed vector insert is illustrated in FIG. 27. It consists of the Cre gene for the site specific recombinase under the expression control of the araBAD promoter (PBAD). Expression of PBAD is both positively and negatively regulated by the product of the araC gene (Ogden et al. 1980, Proceedings of the National Academy of Sciences USA 77: 3346-3350), a transcriptional regulator that forms a complex with L-arabinose. When arabinose is not present, a dimer of AraC dimer forms a 210 bp DNA loop by bridging the O₂ and I₁ sites of the araBAD operon. Maximum transcriptional activation occurs when arabinose binds to AraC. This releases the protein from the O₂ site, which now binds the I₂ site adjacent to the I₁ site. This liberates the DNA loop and allows transcription to begin (Soisson et al. 1997, Science 276: 421-425). The binding of AraC to I1 and I2 is facilitated by the cAMP activator protein (CAP)-cAMP complex binding to the DNA. Repression of basal expression levels can be enhanced by introducing glucose to the growth medium. Glucose acts by lowering cAMP levels, which in turn decreases the binding of CAP. As cAMP levels are lowered, transcriptional activation is decreased, which is necessary when expression of the protein of interest is undesirable (Hirsh et al. 1977, Cell 11: 545-550).

The vector insert also contains a T-DNA region for Agrobacterium-mediated gene transfer consisting of a T-DNA border and overdrive sequences flanked by the nopaline synthase promoter (pNOS) on one side and the NPTII coding region and nopaline synthase 3′ terminator on the other side. The T-DNA region is bound by LoxP sites at each end. Although this T-DNA could be transferred to plant cells upon Agrobacterium-mediated transformation, transformed cells cannot be selected since the components of the selectable marker gene (NPTII) are disorganised resulting in a non-functional gene; the promoter is downstream of the coding and 3′ terminator regions.

Induction of Cre recombinase effects site specific recombination between the two LoxP sites, thereby generating a small T-DNA minicircle. This recombination event also generates an intact functional selectable marker gene by orientating the nopaline synthase promoter upstream of the NPTII coding region. During Agrobacterium-mediated transformation from this minicircle, T-strand formation is initiated from the T-DNA border and limited to only the DNA on the minicircle. Selection for transformation events based on the functional selectable marker gene that is only generated upon minicircle formation will ensure the recovery of transformed plants from the well-defined minimal T-DNA region without the inadvertent transfer of vector backbone sequences.

The nopaline synthase promoter was excised as a PstI-BglII fragment from pMOA33 (Barrell and Conner 2006, BioTechniques, 41: 708-710) and ligated between LoxP66 and the T-DNA border/overdrive of p7LoxP (see Example 3A) to give p7LoxPN. The NPTII coding region with the nopaline synthase 3′ region terminator was excised as 1113 bp ApaI-ClaI fragment from pMOA33 (Barrel and Conner 2006, BioTechniques, 41: 708-710) and ligated between the T-DNA border/overdrive and LoxP71 of p7LoxPN to produce p7LoxPNKan.

The 1945 bp NotI fragment from p7LoxPNKan comprising the minicircle forming T-DNA region was cloned into the NotI site of pART27MCS (see Example 3A). The resulting plasmid was restricted with XbaI and blunt ligated with the 2477 bp SphI-PmeI fragment comprising the araBAD-Cre cassette from pBAD202DtopoCre (FIG. 23), following the treatment of both fragments with the Quick Blunting Kit (NEB, Beverly, Mass., USA). The completed plasmid was designated pMOA40. The full sequence of the region cloned onto the 8235 bp backbone of pART27MCS is shown in SEQ ID NO: 21, where:
nucleotides 1-6 represent the Sail restriction enzyme recognition site from pART27MCS;
nucleotides 7-97 represent vector sequence from pART27MCS consisting of restriction enzyme recognition sites for Sad (nucleotides 74-79) and NotI (nucleotides 90-97);
nucleotides 98-131 represent the LoxP site loxP66;
nucleotides 132-137 represent the BglII restriction enzyme recognition site;
nucleotides 133-756 represent the nopaline synthase promoter;
nucleotides 752-757 represent the PstI restriction enzyme recognition site;
nucleotides 758-835 represent a multiple cloning site from pUC57LoxP consisting of restriction enzyme recognition sites for HindIII, AatII, Acc651/KpnI, SpeI, Bsp1407I, SmalI/XmaI, EcoRI, AccIII, MfeI, SplI, SacI, XhoI and AvrII;
nucleotides 836-860 represent a T-DNA border sequence from Agrobacterium;
nucleotides 861-884 represent the overdrive sequence from Ti plasmid of Agrobacterium (octopine strains);
nucleotides 885-890 represent the ClaI restriction enzyme recognition site;
nucleotides 887-1999 represent the nopaline synthase 3′ terminator region (nucleotides 887-1190) and the neomycin phosphotransferase II (NPTII) coding region (nucleotides 1191-1994) on a 1119 bp ClaI-ApaI fragment;
nucleotides 1995-2000 represent the ApaI restriction enzyme recognition site;
nucleotides 2001-2034 represent the LoxP site loxP71;
nucleotides 2035-2042 represent the NotI restriction enzyme recognition site;
nucleotides 2043-2048 represent the XbaI restriction enzyme recognition site;
nucleotides 2048-4524 represent the arabinose-inducible Cre recombinase under control of the araBAD promoter on a blunted 2477 bp SphI-PmeI fragment, consisting of the Cre recombinase coding region (nucleotides 2148-3179), araBAD promoter and regulatory elements (nucleotides 3256-3528) and the araC gene (nucleotides 3558-4436);
nucleotides 4525-4529 represent the blunted XbaI restriction enzyme recognition site;
nucleotides 4530-4607 represent vector sequence from pART27MCS consisting of restriction enzyme recognition sites for SpeI, BamHI, SmaI/XmaI, PstI, EcoRI, EcoRV, HindIII, ClaI, SalI, XhoI, ApaI and KpnI; and
nucleotides 4608-12661 represent vector backbone of pART27MCS.

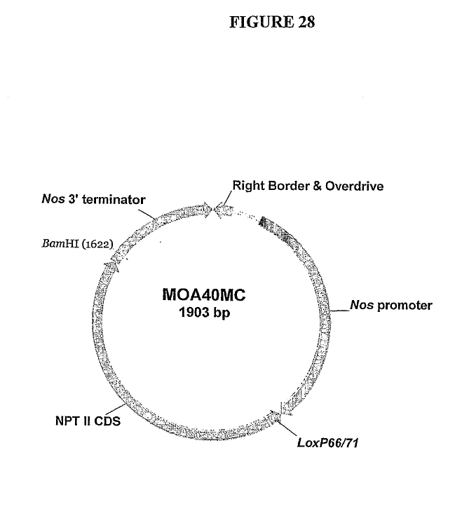

When the binary vector pMOA40 is propagated in Escherichia coli or Agrobacterium, the presence of arabinose induces the expression of Cre recombinase which results in intramolecular recombination between the loxP66 and loxP71 sites and produces a T-DNA minicircle and a residual plasmid of the remaining sequences. The T-DNA minicircle is illustrated in FIG. 28 and defines a minimal unit from which a well defined T-strand can be synthesised, without vector backbone sequences, during Agrobacterium-mediated gene transfer. The full sequence of this minicircle, MOA40MC, is shown in SEQ ID NO: 22, where:
nucleotides 1-24 represent the overdrive sequence from Ti plasmid of Agrobacterium (octopine strains);
nucleotides 25-49 represent a T-DNA border sequence from Agrobacterium with T-strand expected to initiate about nucleotide 47 (see arrow);
nucleotides 50-139 represent a multiple cloning site from pUC57LoxP consisting of restriction enzyme recognition sites for AvrII, XhoI, SacI, SplI, MfeI, AccIII, EcoPJ, SmaI/XmaI, Bsp14071, SpeI, Acc65I/KpnI and AatII;
nucleotides 140-753 represent the nopaline synthase promoter;
nucleotides 754-787 represent a recombined LoxP site with nucleotides 754-769 originating from loxP66 and nucleotides 771-787 originating from loxP71;
nucleotides 788-1903 represent the neomycin phosphotransferase II (NPTII) coding region (nucleotides 794-1597) and the nopaline synthase 3′ terminator region (nucleotides 1598-1896).

Following arabinose induction of the minicircle from pMOA40, the presence of minicircles can be conveniently verified by restricting plasmid preparations with BamHI. The 12,661 bp parent plasmid pMOA40 gives rise to fragments of 9287, 1657, 1248, and 469 bp. The T-DNA minicircle produces a 1903 bp fragment and the recombined plasmid backbone results in 9041, 1248, and 469 bp fragments. As expected, overnight cultures of Escherichia coliDH5α with pMOA40 in LB plus 100 μg/ml spectinomycin and 0.2% glucose failed to, produce minicircles. From this overnight culture, 10 μl was transferred to fresh LB medium with 100 μg/ml spectinomycin, grown for 2 hours at 37° C. and 1000 rpm until OD₆₀₀=0.5, then grown in the same medium, or with the addition of 0.2% glucose, 0.002% L-arabinose, 0.02% L-arabinose, 0.2% L-arabinose, 2% L-arabinose or 20% L-arabinose for 4 hours. Minicircles were only observed following 4 hour induction with 20% L-arabinose and 2% L-arabinose, with a trace presence of minicircles following 4 hour induction with 0.2% L-arabinose. No minicircle induction was observed, even in the absence of glucose or less than 0.2% L-arabinose.

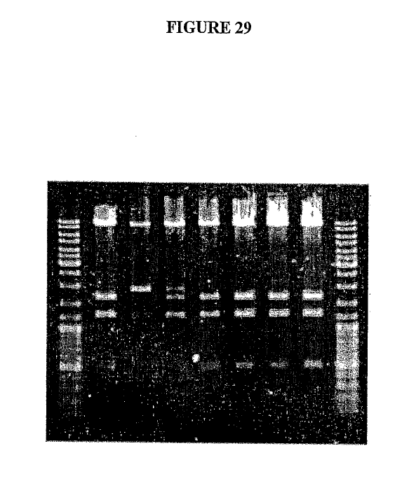

The experiment to confirm the production of minicircles was repeated in overnight cultures of Escherichia coli DH5α with pMOA40. Cultures were incubated in LB plus 100 ng/ml spectinomycin at 1000 rpm overnight at 37° C. with the addition of 0.2%, 2% or 20% L-arabinose or 0.2%, 2% or 20% D-arabinose. Following the restriction of plasmid preparations with BamHI, the induction of minicircles was only evident in the presence of L-arabinose, with very high yields in response to induction 20% L-arabinose (FIG. 29). Most importantly, the presence of the minicircle was stable in overnight cultures and highly recoverable.

The pMOA40 binary vector was transformed into the disarmed Agrobacterium tumefaciens strain EHA105 (Hood et al 1993, Transgenic Research, 2: 208-218), using the freeze-thaw method (Hofgen and Willmitzer 1988, Nucleic Acids Research, 16: 9877). Agrobacterium was cultured overnight at 28° C. in LB broth supplemented with 300 μg/ml spectinomycin and 200 mM L-arabinose and used to transform tobacco (Nicotiana tabacum ‘Petit Havana SR1’), as described in Example 3A.

Genomic DNA was isolated from in vitro shoots of putative transgenic and control plants based on a previously the described method (Bernatzky and Tanksley 1986, Theoretical and Applied Genetics, 72: 314-339). DNA was amplified in a polymerase chain reaction (PCR) containing primers specific for the either the T-DNA minicircle (across the recombined LoxP sites) or the unrecombined T-DNA in the parent binary vector pMOA40. The primer pairs used were:
(i) LOXPMCF1 (^{5′}AGGAAGCGGAACACGTAGAA^{3′}—SEQ ID NO: 23) and LOXPMCR1 (^{5′}GCGGGACTCTAATCATAAAAACC^{3′}—SEQ ID NO: 24) producing an expected product of 1618 bp from the minicircle T-DNA, but no product from the parent plasmid pMOA40 since the primers are orientated in opposite directions;
(ii) LOXPMCF2 (^{5′}GGTTGGGAAGCCCTGCAAAGTAAA^{3′}—SEQ ID NO: 25) and LOXPMCR1 producing an expected product of 1412 bp from the minicircle T-DNA, but no product from the parent plasmid pMOA40 since the primers are orientated in opposite directions;
(iii) CreFor (^{5′}TCTTGCGAACCTCATCACTCGTTG^{3′}—SEQ ID NO: 26) and CreRev (^{5′}CTAATCCCTAACTGCTGGCGGAAA^{3′}—SEQ ID NO: 27) producing an expected product of 166 bp from the parent plasmid pMOA40 but not from the minicircle T-DNA since the sequence is not present.

PCRs were carried out in a Mastercycler (Eppendorf, Hamburg, Germany). The reactions included 10 μl 5× Phusion™ HF Buffer (with 7.5 mM MgCl₂, which provides 1.5 mM MgCl₂ in final reaction conditions), 1 μl dNTP (at 10 mM each of dATP, dCTP, dGTP, dTTP), 0.5 μl Phusion™ High-Fidelity DNA Polymerase at 2 U μ/l (Finnzymes Oy, Espoo, Finland), 0.1 μl of each primer (at 100 μM), 1.0 μl of DNA (10-50 ng) and water to a total volume of 50 μl. The conditions for PCR were: 30 s at 98° C., followed by 30 cycles of 10 s 98° C., 30 s 58° C., 45 s 72° C., followed by a 10 min extension at 72° C. Amplified products were separated by electrophoresis in a 1% agarose gel and visualized under UV light after staining with ethidium bromide.

From the first transformation experiment, five independently regenerated kanamycin-resistant tobacco plants were confirmed as being PCR-positive for the expected products when using the LOXPMCF1/LOXPMCR1 and the LOXPMCF2/LOXPMCR1 primer pairs (S1-01, S1-02, S1-03, S1-04, and S1-05). These plants were therefore derived from the minicircle T-DNA. Four of these plants (S1-02, S1-03, S1-04, and S1-05) were also PCR-positive for the expected products from the CreFor/CreRev primer pair, establishing that they were also co-transformed with the T-DNA from the parent pMOA40 binary vector containing the non-functional NPTII gene.

From a second transformation experiment, thirteen independently regenerated kanamycin-resistant tobacco plants were confirmed as being PCR-positive for the expected 1412 bp product when using the LOXPMCF2/LOXPMCR1 primer pair (JNT01-05, JNT01-09, JNT01-20, JNT01-22, JNT01-25, JNT01-26, JNT01-27, JNT01-29, JNT01-30, JNT01-35, JNT01-39, JNT01-41, and JNT01-44). All of these plants were PCR-negative from the use of the CreFor/CreRev primer pair. These plants were therefore derived from only the minicircle T-DNA.

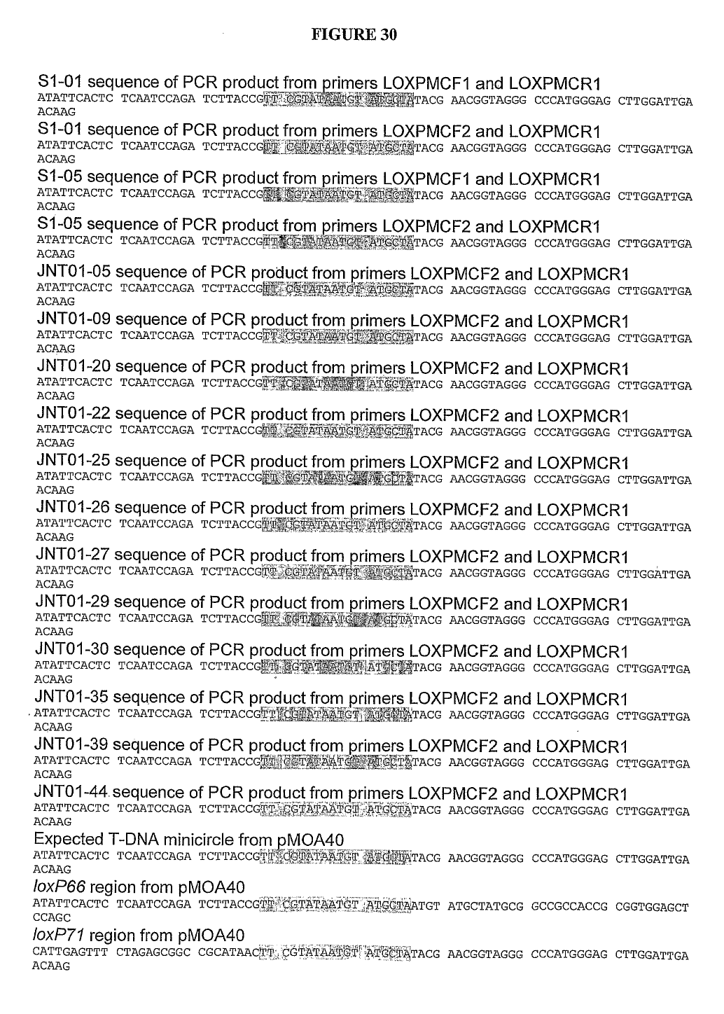

The PCR using the LOXPMCF1/LOXPMCR1 and/or LOXPMCF2/LOXPMCR1 primers pairs generated a product across the intramolecular recombination event between the loxP66 and loxP71 sites. These PCR products were therefore sequenced to verify their authenticity and the fidelity of the arabinose-inducible Cre recombinase event to produce the T-DNA minicircle (FIG. 30). The DNA sequence from fourteen independently transformed tobacco plants (S1-01, S1-05, JNT01-05, JNT01-09, JNT01-20, JNT01-22, JNT01-25, JNT01-26, JNT01-27, JNT01-29, JNT01-30, JNT01-35, JNT01-39, and JNT01-44) and the expected minicircle from pMOA40 are all identical to one another. Furthermore, these sequences are identical to the first part of the sequence from the loxP66 region of pMOA40 and the latter part of the sequence from the loxP71 region from pMOA40. This confirmed that the desired recombination events were induced in Agrobacterium prior to tobacco transformation and were base pair faithful when the minicircles formed.

Eleven transformed plants derived from only the minicircle T-DNA (S1-01, JNT01-09, JNT01-20, JNT01-22, JNT01-25, JNT01-26, JNT01-29, JNT01-30, JNT01-35, JNT01-39, and JNT01-41) were self-pollinated and backcrossed as a pollen and ovule parent to the non-transformed wild-type ‘Petit Havana SR1’ tobacco. The progeny were screened for kanamycin resistance as previously described (Conner et al. 1998, Molecular Breeding, 4: 47-58). The segregation of kanamycin resistance in the self-pollinated progeny of these plants did not deviate from an expected 3:1 ratio as determined by ‘Goodness of Fit’ CM-square tests for all independent pollination events (Table 2). Likewise, in all backcrosses the segregation did not deviate from an expected 1:1 ratio as determined by ‘Goodness of Fit’ Chi-square tests. These results establish that the progeny segregated for kanamycin resistance and kanamycin sensitivity in ratios expected for a single locus insertion of the NPTII gene from the T-DNA minicircle.

TABLE 2
The inheritance of kanamycin resistance in tobacco (Nicotiana tabacum
‘Petit Havana SR1’) following Agrobacterium-mediated
transformation using T-DNA minicircles from pMOA40.

| | | Number of | Number of | | |
|---|---|---|---|---|---|
| | | kanamycin- | kanamycin- | | |
| | | resistant | susceptible | | |
| Plant line | Cross | progeny | progeny | Ratio | Chi-square |
| Wild-type | Selfed | 0 | 183 | 0:1 | — |
| | Selfed | 0 | 142 | 0:1 | — |
| | Selfed | 0 | 227 | 0:1 | — |
| | Selfed | 0 | 313 | 0:1 | — |
| S1-01 | Selfed | 173 | 59 | 3:1 | 0.02 |
| | Selfed | 327 | 101 | 3:1 | 0.45 |
| | Selfed | 279 | 105 | 3:1 | 1.13 |
| | S1-01 × wt | 228 | 244 | 1:1 | 0.54 |
| | S1-01 × wt | 221 | 239 | 1:1 | 0.70 |
| | wt × S1-01R | 240 | 226 | 1:1 | 0.42 |
| JNT1-09 | Selfed | 94 | 30 | 3:1 | 0.04 |
| | Selfed | 99 | 42 | 3:1 | 1.86 |
| | Selfed | 92 | 33 | 3:1 | 0,17 |
| | Selfed | 81 | 22 | 3:1 | 0.21 |
| | 1-09 × wt | 54 | 52 | 1:1 | 0.04 |
| | 1-09 × wt | 59 | 50 | 1:1 | 0.74 |
| | 1-09 × wt | 40 | 49 | 1:1 | 0.91 |
| | wt × 1-09 | 77 | 60 | 1:1 | 1.11 |
| | wt × 1-09 | 87 | 83 | 1:1 | 0.09 |
| | wt × 1-09 | 89 | 71 | 1:1 | 2.03 |
| JNT1-20 | Selfed | 125 | 36 | 3:1 | 0.53 |
| | Selfed | 100 | 30 | 3:1 | 0.26 |
| | Selfed | 108 | 38 | 3:1 | 0.08 |
| | Selfed | 73 | 27 | 3:1 | 0.21 |
| | 1-20 × wt | 60 | 49 | 1:1 | 0.31 |
| | 1-20 × wt | 65 | 45 | 1:1 | 3.64 |
| | 1-20 × wt | 61 | 55 | 1:1 | 0.31 |
| | 1-20 × wt | 51 | 49 | 1:1 | 0.04 |
| | wt × 1-20 | 86 | 75 | 1:1 | 0.75 |
| | wt × 1-20 | 76 | 74 | 1:1 | 0.01 |
| | wt × 1-20 | 83 | 89 | 1:1 | 0.21 |
| JNT1-22 | Selfed | 89 | 29 | 3:1 | 0.01 |
| | Selfed | 106 | 42 | 3:1 | 0.90 |
| | Selfed | 90 | 22 | 3:1 | 1.71 |
| | 1-22 × wt | 70 | 67 | 1:1 | 0.07 |
| | 1-22 × wt | 57 | 56 | 1:1 | 0.01 |
| | 1-22 × wt | 81 | 88 | 1:1 | 0.29 |
| | wt × 1-22 | 50 | 54 | 1:1 | 0.15 |
| JNT1-25 | Selfed | 94 | 36 | 3:1 | 0.50 |
| | Selfed | 101 | 54 | 3:1 | 7.71 |
| | Selfed | 83 | 37 | 3:1 | 2.18 |
| | 1-25 × wt | 55 | 71 | 1:1 | 2.03 |
| | 1-25 × wt | 63 | 56 | 1:1 | 0.41 |
| | 1-25 × wt | 50 | 55 | 1:1 | 0.24 |
| | wt × 1-25 | 79 | 88 | 1:1 | 0.49 |
| | wt × 1-25 | 62 | 65 | 1:1 | 0.07 |
| JNT1-26 | Selfed | 111 | 34 | 3:1 | 0.15 |
| | Selfed | 108 | 44 | 3:1 | 1.26 |
| | 1-26 × wt | 51 | 61 | 1:1 | 0.89 |
| | 1-26 × wt | 65 | 87 | 1:1 | 3.18 |
| | 1-26 × wt | 72 | 77 | 1:1 | 0.17 |
| | wt × 1-26 | 62 | 53 | 1:1 | 0.70 |
| | wt × 1-26 | 51 | 54 | 1:1 | 0.09 |
| JNT1-29 | Selfed | 124 | 28 | 3:1 | 3.51 |
| | Selfed | 97 | 33 | 3:1 | 0.01 |
| | Selfed | 90 | 35 | 3:1 | 0.69 |
| | wt × 1-29 | 52 | 52 | 1:1 | 0.00 |
| | wt × 1-29 | 55 | 55 | 1:1 | 0.00 |
| | wt × 1-29 | 74 | 66 | 1:1 | 0.46 |
| JNT1-30 | Selfed | 106 | 29 | 3:1 | 0.98 |
| | Selfed | 98 | 29 | 3:1 | 0.38 |
| | Selfed | 88 | 23 | 3:1 | 1.19 |
| | Selfed | 98 | 34 | 3:1 | 0.04 |
| | 1-30 × wt | 55 | 50 | 1:1 | 0.24 |
| | 1-30 × wt | 67 | 61 | 1:1 | 0.28 |
| | 1-30 × wt | 54 | 44 | 1:1 | 1.02 |
| | 1-30 × wt | 60 | 64 | 1:1 | 0.13 |
| | wt × 1-30 | 47 | 55 | 1:1 | 0.63 |
| JNT1-35 | Selfed | 92 | 30 | 3:1 | 0.01 |
| | Selfed | 94 | 22 | 3:1 | 2.25 |
| | Selfed | 68 | 25 | 3:1 | 0.27 |
| | Selfed | 82 | 26 | 3:1 | 0.05 |
| | 1-35 × wt | 54 | 45 | 1:1 | 0.82 |
| | 1-35 × wt | 55 | 57 | 1:1 | 0.04 |
| | 1-35 × wt | 48 | 59 | 1:1 | 1.13 |
| | 1-35 × wt | 55 | 70 | 1:1 | 1.80 |
| | wt × 1-35 | 62 | 80 | 1:1 | 2.28 |
| | wt × 1-35 | 53 | 54 | 1:1 | 0.01 |
| JNT1-39 | Selfed | 203 | 71 | 3:1 | 0.12 |
| | 1-39 × wt | 52 | 72 | 1:1 | 3.22 |
| | 1-39 × wt | 97 | 94 | 1:1 | 0.05 |
| JNT1-41 | Selfed | 128 | 32 | 3:1 | 2.13 |
| | Selfed | 97 | 31 | 3:1 | 0.04 |
| | Selfed | 86 | 29 | 3:1 | 0.01 |
| | 1-41 × wt | 79 | 72 | 1:1 | 0.32 |
| | 1-41 × wt | 67 | 50 | 1:1 | 2.47 |
| | wt × 1-41 | 78 | 77 | 1:1 | 0.01 |
| | wt × 1-41 | 77 | 76 | 1:1 | 0.01 |

### Example 4

### Design and Construction of Intragenic T-DNA Potato Minicircles for Agrobacterium-Mediated Gene Transfer

T-DNA constructs were designed to generate intragenic T-DNA minicircles based on potato DNA to allow the transfer of potato genes to potatoes by Agrobacterium-mediated transformation. In this manner the T-strand formation during Agrobacterium-mediated gene transfer can be limited to only intragenic DNA derived from potato, thereby eliminating the opportunity for vector backbone sequences or any other foreign DNA to be transferred to plants.

### (A) A Potato-Derived T-DNA Minicircle Based on a Visual Marker Gene

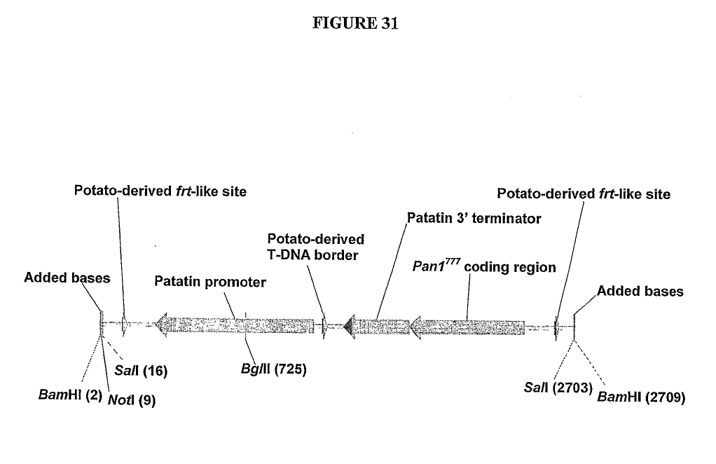

A 2713 bp sequence of DNA composed from a series of DNA fragments derived from potato (Solanum tuberosum) was constructed in silico. This consisted of a potato-derived T-DNA border sequence flanked by the promoter of a potato patatin class I gene on one side and the coding region of a potato myb transcription factor (the D locus allele Stan2⁷⁷⁷) and the 3′ terminator of a patatin class I gene on the other side. This T-DNA region was positioned between a direct repeat of a fragment produced by adjoining two EST's to create a potato-derived frt-like site at their junction. The structure of this potato-derived T-DNA region is illustrated in FIG. 31.

Induction of FLP recombinase effects site specific recombination between the two frt-like sites, thereby generating a small T-DNA minicircle composed entirely of potato DNA. This recombination event also generates an intact functional marker gene by orientating the patatin promoter upstream of the potato myb transcription factor coding region. Expression of this chimeric potato gene induces the biosynthesis of anthocyanins upon transformation of potato tissue. During Agrobacterium-mediated transformation from this minicircle, T-strand formation is initiated from the T-DNA border and limited to only the potato-derived DNA on the minicircle. Potato transformation events identified based on the functional marker gene generated with minicircle formation ensures the recovery of transformed plants from the well-defined minimal T-DNA region without the inadvertent transfer of vector backbone sequences based on foreign DNA.

The potato-derived T-DNA region had the sequence shown in SEQ ID NO: 28, where:
nucleotides 1-6 are added to create a BamHI restriction site as a option for future cloning;
nucleotides 7-14 are added to create a NotI restriction site as a option for future cloning;
nucleotides 15-20 are added to create a Sail restriction site as a option for future cloning;
nucleotides 21-120 represent a potato-derived DNA sequence composed of two adjoining two EST's (nucleotides 21-70 originating from nucleotides 471-520 of NCBI accession CK272589; nucleotides 71-120 originating from the reverse complement of nucleotides 447-496 from NCBI accession BM112095) to create a frt-like site from nucleotides 145-178;
nucleotides 121-1185 are from the patatin class I promoter (reverse complement of nucleotides 41792-42856 of NCBI accession DQ274179);
nucleotides 1186-1385 represent a potato-derived T-DNA border region composed of two adjoining two EST's (nucleotides 1186-1253 originating the reverse complement of nucleotides 121-188 of NCBI accession BE924124; nucleotides 1254-1385 originating from the reverse complement of nucleotides 213-344 from NCBI accession BG889577) to create a T-DNA border from nucleotides 1247-1271;
nucleotides 1386-1824 are from the patatin class I 3′ terminator sequence (originating from the reverse complement of nucleotides 3591-4029 of NCBI accession M18880;
nucleotides 1825-2601 represent the coding region of a myb transcription factor, the D locus allele Stan2⁷⁷⁷, from NCBI accession AY841129 with the addition of the first two codons of the open reading frame (Jung et al. 2009, Theoretical and Applied Genetics, 120: 45-57);
nucleotides 2602-2701 represent a potato-derived DNA sequence composed of two adjoining two EST's (nucleotides 2602-2651 originating from nucleotides 471-520 of NCBI accession CK272589; nucleotides 2652-2701 originating from the reverse complement of nucleotides 447-496 from NCBI accession BM112095) to create a frt-like site from nucleotides 2636-2669;
nucleotides 2702-2707 are added to create a SalI restriction site as a option for future cloning.
nucleotides 2708-2713 are added to create a BamHI restriction site as a option for future cloning.

This 2713 bp potato-derived sequence was synthesised by Genscript Corporation (Piscatawa, N.J., www.genscript.com) and cloned into pUC57 to give pUC57POTIV10. The region from nucleotides 21-2707 is composed entirely of DNA sequences derived from potato and has been verified by DNA sequencing between the M13 forward and M13 reverse universal primers. All subsequent plasmid constructions were performed using standard molecular biology techniques of plasmid isolation, restriction, ligation and transformation into Escherichia coli strain DH5α, unless otherwise stated (Sambrook et al. 1987, Molecular Cloning: A Laboratory Manual, 2^{nd} ed., Cold Spring Harbor Press).

The coding region of the cytosine deaminase (codA) negative selection marker gene [Stougaard 1993, The Plant Journal 3: 755-61] was cloned into pART7 (Gleave 1992, Plant Molecular Biology, 20: 1203-1207) to yield pART8codA. This placed codA under the regulatory control of the 35S promoter and the octopine synthase 3′ terminator region, which was then cloned as a NotI fragment into the NotI site of pUC57POTIV10 to give pUC57POTIV10codA.

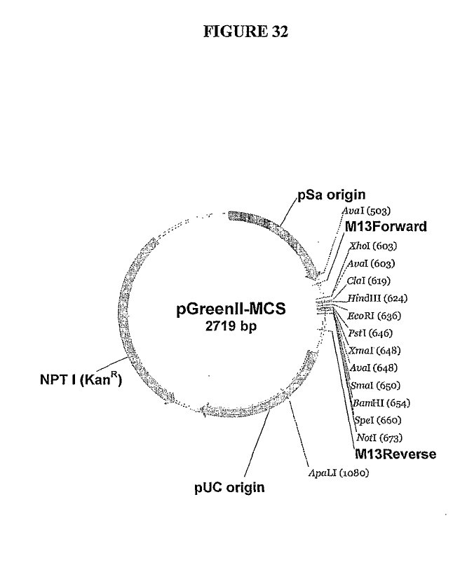

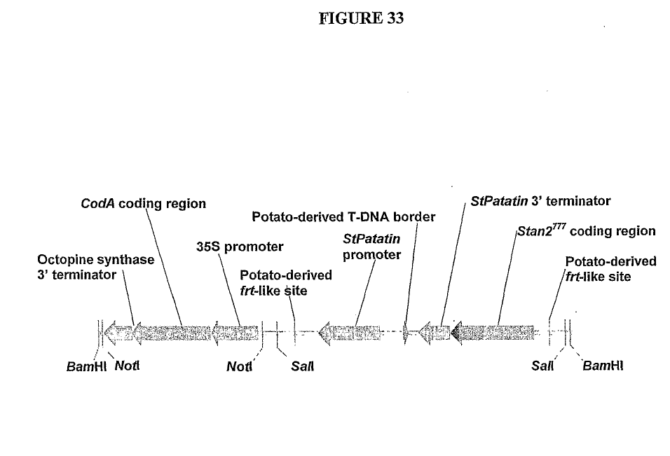

The T-DNA region of pGreen0000 (Hellens et al. 2000, Plant Molecular Biology, 42: 819-832) bound by BglII restriction enzyme recognition sites was replaced with the multiple cloning site from pBLUESCRIPT to yield pGreenII-MCS (FIG. 32). The BamHI fragment of pUC57POTIV10codA was then cloned into the BamHI site of pGreenII-MCS to yield pPOTIV10. The complete T-DNA region pPOTIV10 is illustrated in FIG. 33. The presence of the codA negative selection marker gene prevents to recovery of any transformed plants originating from the parent T-DNA of pPOTIV10 prior to minicircle formation.

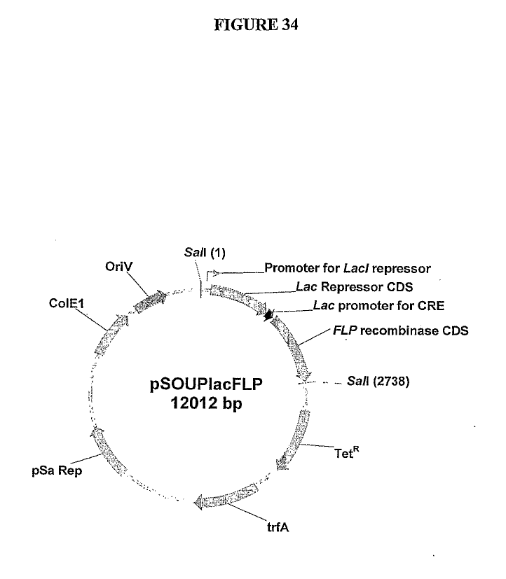

The induction of minicircles in E. coli or Agrobacterium can be achieved by the expression of the FLP recombinase gene under an inducible promoter such as the Lac promoter. The vector backbone of pGreen vector series requires the presence of an additional helper plasmid, pSOUP, to enable the binary vector to replicate in Agrobacterium (Hellens et al. 2000, Plant Molecular Biology, 42: 819-832; Hellens et al. 2005, Plant Methods 1:13). Therefore, cloning the inducible FLP construct into pSOUP conveniently provides the FLP recombinase gene in trans to the binary vector containing the T-DNA forming minicircle. To achieve this, the FLP coding region was PCR amplified from genomic DNA of Escherichia coli strain 294-FLP (Buchholz et al. 1996, Nucleic Acids Research, 24: 3118-3119) using high fidelity Vent polymerase (NEB, Beverly, Mass., USA). Similarly, the Lac promoter region, including the Lad gene, was PCR isolated from pUC57LacICre (Plant & Food Research). The FLP coding region was then cloned under the control of the inducible Lac promoter in pART27MCS (see Example 3A). The inducible Lac-FLP cassette was then cloned as a SalI fragment into pSOUP to give pSOUPLacFLP (FIG. 34).

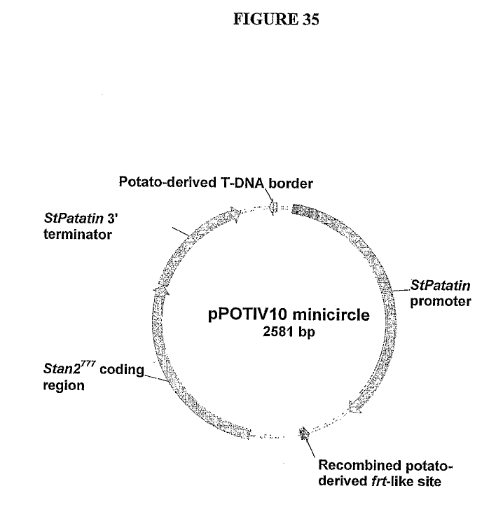

The transfer of pSOUPLacFLP and pPOTIV10 into the same Agrobacterium cell provides the inducible FLP recombinase gene in trans to the binary vector containing the T-DNA forming minicircle. Selection for the presence of the codA negative selection marker gene on pPOTIV10 prevents to recovery of any transformed plants originating from the parent T-DNA of pPOTIV10 prior to minicircle formation. This provides a convenient system to ensure effective intragenic transformation of potato without the inadvertent transfer of vector backbone sequences. This provides a convenient system to ensure effective intragenic transformation of potato without the inadvertent transfer of vector backbone sequences. The 2581 bp potato ‘POTIV10’ minicircle is composed entirely of DNA fragments derived from potato and contains a chimeric gene anticipated to induce the biosynthesis of anthocyanins (FIG. 35). The full sequence of the potato ‘POTIV10’ minicircle is shown in SEQ ID NO: 29, where:
nucleotides 1-200 represent a potato-derived T-DNA border region composed of two adjoining EST's (nucleotides 1-132 originating from nucleotides 213-344 from NCBI accession BG889577; nucleotides 133-200 originating the reverse complement of nucleotides 121-188 of NCBI accession BE924124) to create a T-DNA border from nucleotides 115-139;
nucleotides 201-1265 are from the patatin class I promoter (nucleotides 41792-42856 of NCBI accession DQ274179);
nucleotides 1266-1315 originate from nucleotides 447-496 from NCBI accession BM112095;
nucleotides 1316-1365 originate from the reverse complement of nucleotides 471-520 of NCBI accession CK272589;
nucleotides 1298-1331 represent the FLP-induced recombined potato-derived frt-like site;
nucleotides 1366-2142 represent the coding region of a myb transcription factor, the D locus allele Pan1⁷⁷⁷, from WO 2006/062698;
nucleotides 2143-2581 are from the patatin class I 3′ terminator sequence (originating from the reverse complement of nucleotides 3591-4029 of NCBI accession M18880.

### (B) A Potato-Derived T-DNA Minicircle Based on a Selectable Marker Gene

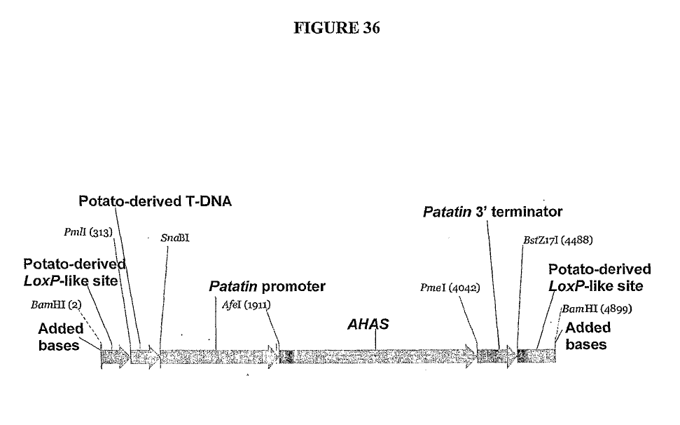

A 4903 bp sequence of DNA composed from a series of DNA fragments derived from potato (Solanum tuberosum) flanked by BamHI restriction sites was constructed in silico. This consisted of a potato-derived T-DNA border sequence flanked by direct repeats of potato-derived LoxP-like sites. A potato-derived chimeric selectable marker gene was positioned between the potato-derived T-DNA border and one potato-derived LoxP site. This marker gene consisted of the coding region of a potato acetohydroxyacid synthase (AHAS) gene under the transcriptional control of the promoter and 3′ terminator of a potato patatin class I gene. The AHAS coding region carried two point mutations conferring tolerance to the sulfonylurea herbicides isolated from chlorsulfuron-tolerant potato plants originally derived through somatic cell selection in the cultivar Iwa. The structure of this potato-derived T-DNA region is illustrated in FIG. 36.

Induction of Cre recombinase results in site specific recombination between the two LoxP-like sequences, thereby generating a small T-DNA minicircle composed entirely of potato DNA. During Agrobacterium-mediated transformation from this minicircle, T-strand formation is initiated from the T-DNA border and limited to only the potato-derived DNA on the minicircle. The potato-derived T-DNA region had the sequence shown in SEQ ID NO: 30, where:
nucleotides 1-4 are added to create a BamHI restriction site as a option for future cloning;
nucleotides 5-312 represent a potato-derived DNA sequence composed of two adjoining two EST's (nucleotides 5-133 originating from the reverse complement of nucleotides 17-145 of NCBI accession BQ111407; nucleotides 134-312 originating from the reverse complement of nucleotides 370-548 of NCBI accession BQ045786) to create a LoxP-like site from nucleotides 115-148;
nucleotides 313-632 represent a potato-derived T-DNA border region composed of two adjoining EST's (nucleotides 313-425 originating the reverse complement of nucleotides 121-233 of NCBI accession BE924124; nucleotides 426-632 originating from the reverse complement of nucleotides 138-344 from NCBI accession B0889577) to create a T-DNA border from nucleotides 419-443;
nucleotides 633-1910 are from the patatin class I promoter (reverse complement of nucleotides 41542-42819 of NCBI accession DQ274179);
nucleotides 1911-4041 represent the coding region of an AHAS gene from potato cultivar Iwa with two point mutations (C to T at nucleotide 2530 resulting in an amino acid substitution from proline to serine and T to A at nucleotide 3661 resulting in an amino acid substitution from tryptophan to arginine);
nucleotides 4042-4487 are from the patatin class I 3′ terminator sequence (originating from nucleotides 3575-4020 of NCBI accession M18880) nucleotides 4488-4900 represent a potato-derived DNA sequence composed of two adjoining two EST's (nucleotides 4488-4717 originating from the reverse complement of nucleotides 17-246 of NCBI accession BQ111407; nucleotides 4718-4900 originating from the reverse complement of nucleotides 366-548 from NCBI accession BQ045786) to create a LoxP-like site from nucleotides 4699-4732; and
nucleotides 4901-4903 are added to create a BamHI restriction site as a option for future cloning.

This sequence was synthesised by Genscript Corporation (Piscatawa, N.J., USA, www.genscript.com) and cloned into pUC57 to give pUC57POTIV11. The inserted sequence has been verified by DNA sequencing between the M13 forward and M13 reverse universal primers. All subsequent plasmid constructions were performed using standard molecular biology techniques of plasmid isolation, restriction, ligation and transformation into Escherichia coli strain DH5α (Sambrook et al. 1987, Molecular Cloning: A Laboratory Manual, 2^{nd} ed., Cold Spring Harbor Press). The 4897 bp BamHI fragment from pUC57POTIV11 was cloned into the BamHI site of pGreenII-MCS (FIG. 32) to yield pGreenPOTIV11. The NotI fragment of pART8codA (see Example 31) with codA under the regulatory control of the 35S promoter and the octopine synthase 3′ terminator region was then cloned into the NotI site of pGreenPOTIV11 to give pPOTIV11.

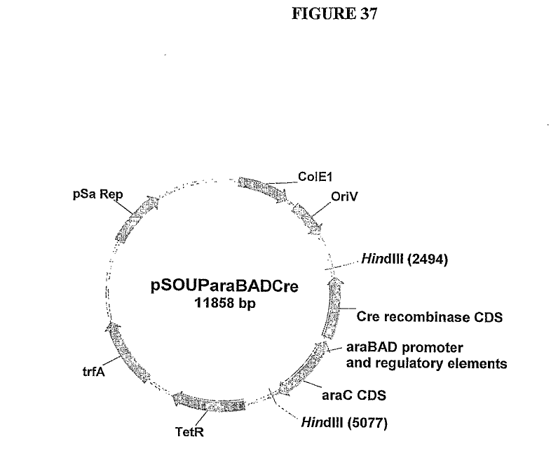

The induction of minicircles from pPOTIV11 in E. coli or Agrobacterium can be achieved by the expression of Cre recombinase under an inducible promoter such as the L-arabinose inducible system described in Example 3. The vector backbone of pGreen vector series requires the presence of an additional helper plasmid, pSOUP, to enable the binary vector to replicate in Agrobacterium (Hellens et al. 2000, Plant Molecular Biology, 42: 819-832; Hellens et al. 2005, Plant Methods 1:13). Therefore, cloning the inducible Cre construct into pSOUP conveniently provides the Cre recombinase gene in trans to the binary vector containing the T-DNA forming minicircle. To achieve this, the 2583 bp HindIII fragment from pMOA38 (Example 3A) containing the Cre recombinase coding region under arabinose-inducible expression was cloned into the HindIII site of pSOUP to give pSOUParaBADCre (FIG. 37).

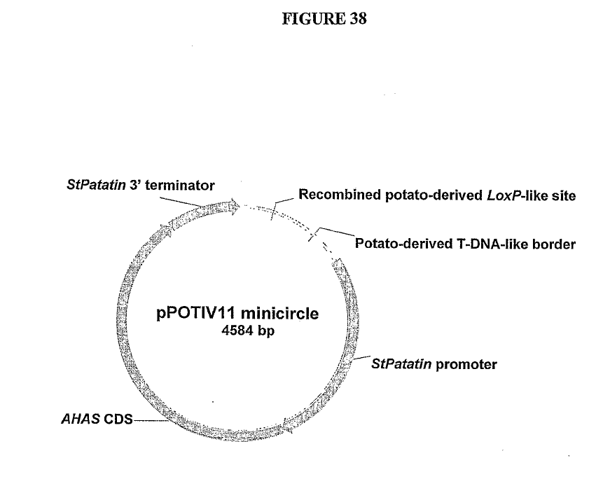

The transfer of pSOUParaBADCre and pPOTIV11 into the same Agrobacterium cell provides the inducible Cre recombinase gene in trans to the binary vector containing the T-DNA forming minicircle. Selection for the presence of the codA negative selection marker gene on pPOTIV11 prevents to recovery of any transformed plants originating from the parent T-DNA of pPOTIV11 prior to minicircle formation. This provides a convenient system to ensure effective intragenic transformation of potato without the inadvertent transfer of vector backbone sequences. The 4584 bp potato ‘POTIV11’ minicircle is composed entirely of DNA fragments derived from potato and contains a chimeric selectable marker gene conferring resistance to chlorsulfron (FIG. 38). The full sequence of the potato ‘POTIV11’ minicircle is shown in SEQ ID NO: 31, where:
nucleotides 1-409 represent a potato-derived DNA sequence composed of two adjoining two EST's (nucleotides 1-230 originating from the reverse complement of nucleotides 17-246 of NCBI accession BQ111407; nucleotides 231-409 originating from the reverse complement of nucleotides 366-548 from NCBI accession BQ045786)
nucleotides 212-245 represent the Cre-induced recombined potato-derived LoxP-like site;
nucleotides 410-729 represent a potato-derived T-DNA border region composed of two adjoining EST's (nucleotides 410-522 originating the reverse complement of nucleotides 121-233 of NCBI accession BE924124; nucleotides 523-729 originating from the reverse complement of nucleotides 138-344 from NCBI accession BG889577) to create a T-DNA border from nucleotides 516-540;
nucleotides 730-2007 are from the patatin class I promoter (reverse complement of nucleotides 41542-42819 of NCBI accession DQ274179);
nucleotides 2008-4138 represent the coding region of an AHAS gene from potato cultivar Iwa with two point mutations (C to T at nucleotide 2530 resulting in an amino acid substitution from proline to serine and T to A at nucleotide 3661 resulting in an amino acid substitution from tryptophan to arginine);
nucleotides 4139-4584 are from the patatin class I 3′ terminator sequence (originating from nucleotides 3575-4020 of NCBI accession M18880)

The pPOTIV11 and pSOUParaBAD-Cre plasmids were transformed into the disarmed Agrobacterium tumefaciens strain EHA105 (Hood et al 1993, Transgenic Research, 2: 208-218), using the freeze-thaw method (Hofgen and Willmitzer 1988, Nucleic Acids Research, 16: 9877). Agrobacterium habouring the two plasmids was cultured overnight at 28° C. in LB broth supplemented with 50 μg/ml kanamycin and 200 mM L-arabinose and used to transform potato (Solanum tuberosum ‘Iwa’).

Virus-free plants of cultivar Iwa were multiplied in vitro on a multiplication medium consisting of MS salts and vitamins (Murashige & Skoog 1962, Physiologia Plantarum, 15: 473-497) plus 30 g/l sucrose, 40 mg/l ascorbic acid, 500 mg/l casein hydrolysate, and 7 g/l agar. The agar was added after pH was adjusted to 5.8 with 0.1 M KOH, then the medium was autoclaved at 121° C. for 15 min. Then 50 ml was dispensed into (80 mm diameter×50 mm high) pre-sterilised plastic containers (Vertex Plastics, Hamilton, New Zealand). Plants were routinely subcultured as two to three node segments every 3-4 weeks and incubated at 26° C. under cool white fluorescent lamps (80-100 μmol/m²/s; 16-h photoperiod).

Fully expanded leaves from the in vitro plants were excised, cut in half across midribs, while submerged in the liquid Agrobacterium culture. After about 30 sec, these leaf segments were blotted dry on sterile filter paper (Whatman® No. 1, 100 mm diameter). They were then cultured on callus induction medium (multiplication medium without the casein hydrolysate, but supplemented with 0.2 mg/l napthaleneactic acid and 2 mg/l benzylaminopurine) in standard plastic Petri dishes (9 cm diameter×1 cm high) under reduced light intensity (5-10 μmol/m²/s) by covering the Petri dishes with white paper. After two days, the leaf segments were transferred to the callus induction medium supplemented with 200 mg/l Timentin™ (filter sterilised and added after autoclaving) to prevent Agrobacterium overgrowth. Five days later, they were transferred on to the same medium further supplemented with 10 μg/l chlorsulfuron (filter sterilised and added after autoclaving) in order to select the transformed cell colonies. Individual chlorsulfuron-tolerant cell colonies (0.5-1 mm diameter), developing on the leaf segments in 3-6 weeks, were excised and transferred on to regeneration medium (potato multiplication medium without the casein hydrolysate and with sucrose reduced to 5 g/l, plus 1.0 mg/l zeatin and 5 mg/l GA₃, both filter sterilised and added after autoclaving) supplemented with 200 mg/l Timentin and 10 μg/1 chlorsulfuron in plastic Petri dishes (9 cm diameter×2 cm high). These were cultured under low light intensity (30-40 μmol/m²/s) until shoots regenerated. A single healthy shoot derived from individual cell colonies were excised and transferred to multiplication medium containing 100 mg l⁻¹ Timentin for recovery of transformed plants. The addition of 200 mg/15-fluorocytosine along with the chlorsulfuron ensured recovery of plants only derived from the ‘POTIV11’ minicircle.

### Example 5

### Design, Construction and Verification of Plant Derived Recombination Sites: loxP-Like Sites for Recombination with Cre Recombinase

BLAST searches were conducted of publicly available plant DNA sequences from NCBI, SGN and TIGR databases.

### 1) Potato DNA Fragment Containing a LoxP-Like Sequence—PotLoxP

A fragment containing a loxP-like sequence was designed from two EST sequences from potato (Solanum tuberosum) (NCBI accessions BQ111407 and BQ045786). This fragment, named POTLOXP, is illustrated below. Restriction enzyme sites used for DNA cloning into the potato intragenic T-DNA described in Example 8 are shown in bold and the loxP-like sequence shown in bold and light grey.

| (SEQ ID NO: 32) | |
|---|---|
| 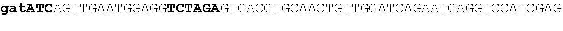 | |
| 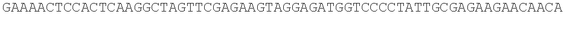 | |
| 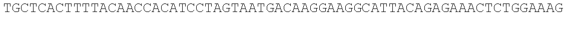 | |
| 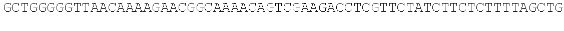 | |
| 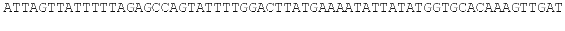 | |
| 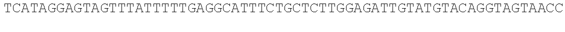 | |
| 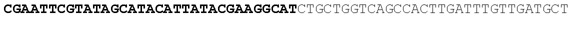 | |
| 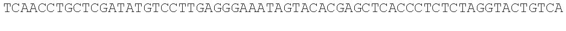 | |
| 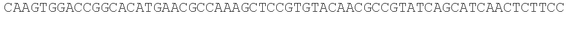 | |
| 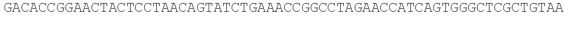 | |
| 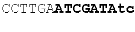 | |
| Nucleotides 1-3 | part of EcoRV restriction enzyme site (from |
| | the potato intragenic vector pPOTINV) |
| Nucleotides 4-402 | nucleotides 17-415 of NCBI accession BQ111407 |
| Nucleotides 403-653 | nucleotides 298-548 of NCBI accession |
| | BQ045786 |
| Nucleotides 654-655 | part of EcoRV restriction enzyme site (from |
| | the potato intragenic |
| | T-DNA) |

The designed potato loxP-like sequence has 6 nucleotide mismatches from the native loxP sequence as illustrated in bold below.

| | |
|---|---|
| | (SEQ ID NO: 33) |
| loxP sequence | ATAACTTCGTATAGCATACATTATACGAAGTTAT |
| | (SEQ ID NO: 34) |
| Potato loxP-like | 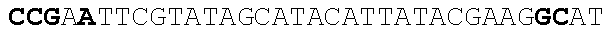 |

The 655 bp POTLOXP sequence illustrated above was synthesised by Genscript Corporation (Piscatawa, N.J., www.genscript.com) and supplied cloned into pUC57. All plasmid constructions were performed using standard molecular biology techniques of plasmid isolation, restriction, ligation and transformation into Escherichia coli strain DH5α (Sambrook et al., Molecular Cloning: A Laboratory Manual, 2nd Ed. Cold Spring Harbor Press, 1987).

Initially the 1286 bp SalI fragment encompassing the T-DNA composed of potato DNA from pUC57POTINV was subcloned into pGEMT to form pGEMTPOTINV. POTLOXP was then cloned into pGEMTPOTINV twice, firstly as a XbaI to ClaI fragment, then subsequently as a EcoRV to EcoRV fragment. Confirmation of the POTLOXP inserts was verified using restriction enzyme analysis and DNA sequencing. The resulting plasmid was named pPOTLOXP2.

The DNA sequence of the 2316 bp SalI fragment comprising the potato derived T-DNA region in pPOTLOXP2 is illustrated below. Only the nucleotides in italics are not part of potato genome sequences. The POTLOXP regions are shaded. The T-DNA borders are shown in bold, with the left border positioned at 314-337 and the right border positioned at. 2005-2028. Restriction sites illustrated in bold represent those used in cloning the POTLOXP regions into pGEMTPOTINV. Unique restriction sites in pPOTLOXP2 for cloning between POTLOXP sites are:

| | |
|---|---|
| aflII | C/TTAAG |
| AgeI | A/CCGGT |
| BamHI | G/GATCC |
| BstD102I | GAG/CGG |
| CspI | CG/GWCCG |
| PinAI | A/CCGGT |

(SEQ ID NO: 35)
GTCGACAGTAAAAGTTGCACCTGGAATAAGGTTTTCATTCTTCACAGGAGGCATCTCACTCTTT
CTAGCAGGTCTTGAACGCTTAGATTGAACAGATGTAGGACTCACATCTGATATGGAGGATTCTT
GACTTGTTTCAGCAGCATCAGATGAAGCTTCTGAGACTTCACCTGATCCATCATCTGTAGCAGT
TGCTTCTACTTCTTCCACTGCTACATCAGTCTCAGTTGCTGATACTATAAGACCTCTTAATTTA
GGTCGTAAAATGCAACCAACTCTAAAATGGGGAAACAATTTAATAGATGTTGACAGAGGCAGGA
TATATTTTGGGGTAAACGGGAATTCTTCAGCAGTTGCTCGAGGGAGATTGGCGGTGCTTTCAGC

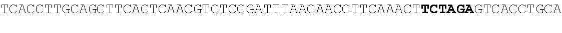

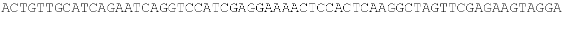

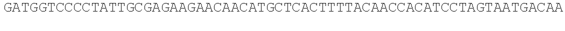

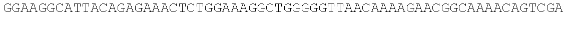

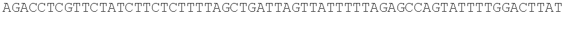

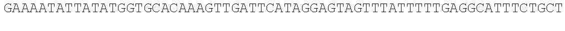

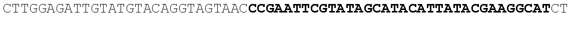

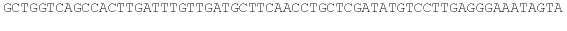

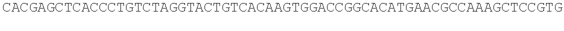

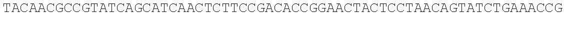

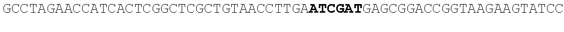

GGTTCAGGTTTCTGAGGATGGCACTATCAAAGCCACCGACTTAAAGAAGATAACAACAGGACAG
AATGATAAAGGTCTTAAGCTTTATGATCCAGGCTATCTCAACACAGCACCTGTTAGGTCATCAA

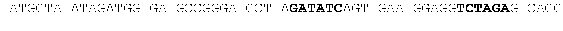

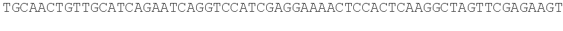

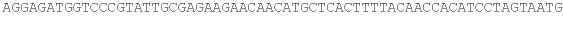

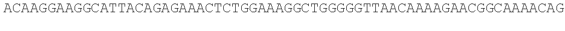

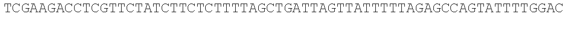

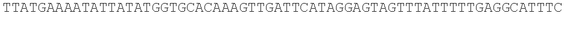

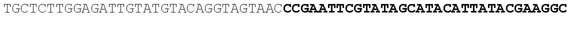

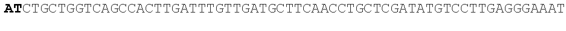

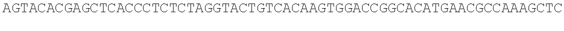

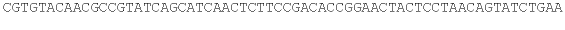

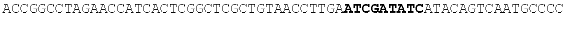

ATGATGCTCATCCAATGGGGGTTCTTGTCAGTGCAATGAGTGCTCTTTCCGTTTTTCATCCTGA
TGCAAATCCAGCTCTGAGAGGACAGGATATATACAAGTGTAAACAATTTAAAAGCATATGGTGG
CACTGCTCAATATATGAGGTGGGCGCGAGAAGCAGGTACCAATGTGTCCTCATCAAGAGATGCA
TTCTTTACCAATCCAACGGTCAAAGCATACTACAAGTCTTTTGTCAAGGCTATTGTGACAAGAA
AAAACTCTATAAGTGGAGTTAAATATTCAGAAGAGCCCGCCATATTTGCGTGGGAACTCATAAA
TGAGCCTCGTTGTGAATCCAGTTCATCAGCTGCTGCTCTCCAGGCGTGGATAGCAGAGATGGCT
GGATTGTCGAC

The ability of this construct to undergo recombination between the POTLOXP sites was tested in vivo using Cre recombinase expressing Escherichia coli strain 294-Cre (Buchholz et al., 1996, Nucleic Acids Research 24 (15) 3118-3119). The binary vector pPOTLOXP2 was transformed into E. coli strain 294-Cre and maintained by selection with 100 mg/l ampillicin and incubation at 23° C. Raising the temperature to 37° C. induces expression of Cre recombinase in E. coli strain 294-Cre, which effected recombination between the two POTLOXP sites in pPOTLOX2. This was evident by a reduction in the size of pPOTLOXP2 from 5316 bp to 4480 pb. Plasmid isolated from colonies of E. coli strain 294-Cre transformed with pPOTLOXP2 and cultured at 37° C., was restricted with SalI. All colonies tested produced the fragments of 3.0 kb and 1.5 kb expected when recombination between the POTLOXP sites has occurred.

Recombination between the POTLOXP sites was further verified by DNA sequencing. Plasmid was isolated from colonies of E. coli strain 294-Cre transformed with pPOTLOXP2 and cultured at 37° C., then DNA sequenced across the SalI region inserted into pGEMT. The resulting sequence from two independent cultures is illustrated below and confirms that recombination is base pair faithful through the remaining POTLOXP site in plasmid preparations. Only the nucleotides in italics are not part of the potato genome sequences. The remaining POTLOXP region is shaded. The T-DNA borders are shown in bold, with the left border positioned at 314-337 and the right border positioned at 1169-1192. Restriction sites illustrated in bold represent those remaining from cloning the POTLOXP regions into pPOTINV.

(SEQ ID NO: 36)
GTCGACAGTAAAAGTTGCACCTGGAATAAGGTTTTCATTCTTCACAGGAGGCATCTCACTCTTT
CTAGCAGGTCTTGAACGCTTAGATTGAACAGATGTAGGACTCACATCTGATATGGAGGATTCTT
GACTTGTTTCAGCAGCATCAGATGAAGCTTCTGAGACTTCACCTGATCCATCATCTGTAGCAGT
TGCTTCTACTTCTTCCACTGCTACATCAGTCTCAGTTGCTGATACTATAAGACCTCTTAATTTA
GGTCGTAAAATGCAACCAACTCTAAAATGGGGAAACAATTTAATAGATGTTGACAGAGGCAGGA
TATATTTTGGGGTAAACGGGAATTCTTCAGCAGTTGCTCGAGGGAGATTGGCGGTGCTTTCAGC

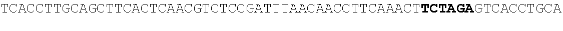

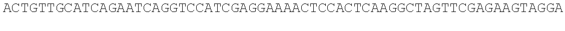

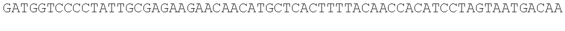

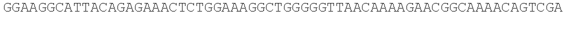

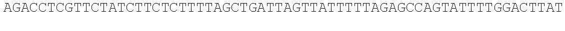

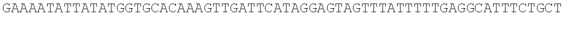

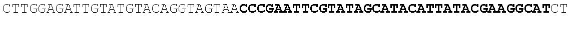

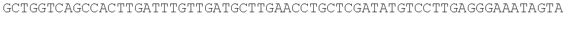

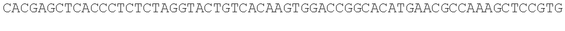

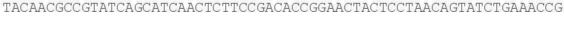

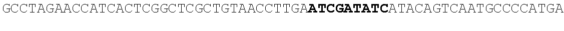

TGCTCATCCAATGGGGGTTCTTGTCAGTGCAATGAGTGCTCTTTCCGTTTTTCATCCTGATGCA
AATCCAGCTCTGAGAGGACAGGATATATACAAGTGTAAACAATTTAAAAGCATATGGTGGCACT
GCTCAATATATGAGGTGGGCGCGAGAAGCAGGTACCAATGTGTCCTCATCAAGAGATGCATTCT
TTACCAATCCAACGGTCAAAGCATACTACAAGTCTTTTGTCAAGGCTATTGTGACAAGAAAAAA
CTCTATAAGTGGAGTTAAATATTCAGAAGAGCCCGCCATATTTGCGTGGGAACTCATAAATGAG
CCTCGTTGTGAATCCAGTTCATCAGCTGCTGCTCTCCAGGCGTGGATAGCAGAGATGGCTGGAT
TTGTCGAC

2) LoxP-Like Sequences from Other Species
Medicago Trunculata (Barrel Medic) LoxP-Like Sequence Designed from 2 ESTs

| | |
|---|---|
| LoxP | |
| ATAACTTCGTATAATGTATGCTATACGAAGTTAT | (SEQ ID NO: 37) |
| Barrel medic loxP-like | |
| ATGACTTCGTATAATGTATGCTATACGAAGTGTG | (SEQ ID NO: 38) |

Nucleotides 1-19
Nucleotides 109-127 of NCBI accession CA919120
Nucleotides 20-34
Nucleotides 14-28 of NCBI accession CA989265

The barrel medic loxP-like site has 4 nucleotide mismatches from the native loxP sequence (illustrated above in bold).

Picea (Spruce) LoxP-Like Sequence Designed from 2 ESTs

| | |
|---|---|
| LoxP | |
| ATAACTTCGTATAATGTATGCTATACGAAGTTAT | (SEQ ID NO: 39) |
| Spruce loxP-like | |
| ATACCTTCGTATAATGTATGCTATACAAAGAAAT | (SEQ ID NO: 40) |

Nucleotides 1-15
Nucleotides 226-240 of NCBI accession CO215992
Nucleotides 16-34
Nucleotides 148-166 of NCBI accession CO255617

The spruce loxP-like site has 4 nucleotide mismatches from the native loxP sequence (illustrated above in bold)

Zea Mays (Maize) LoxP-Like Sequence Designed from 2 ESTs

| | |
|---|---|
| LoxP | |
| ATAACTTCGTATAATGTATGCTATACGAAGTTAT | (SEQ ID NO: 41) |
| Maize loxP-like | |
| GCCACTCCGTATAATGTATGCTATACGAAATGAT | (SEQ ID NO: 42) |

Nucleotides 1-20
Nucleotides 326-345 of NCBI accession CB278114
Nucleotides 21-34
Nucleotides 11-27 of NCBI accession CD001443

The maize loxP-like site has 6 nucleotide mismatches from the native loxP sequence (illustrated above in bold).

### Example 6

### Design, Construction and Verification of Plant Derived Recombination Sites: frt-Like Sites for Recombination with FLP Recombinase

BLAST searches were conducted of publicly available plant DNA sequences from NCBI, SGN and TIGR databases.

### 1) Potato DNA Fragment Containing Aft-Like Sequence—PotFrt

A fragment containing a frt-like sequence was designed from two EST sequences from potato (Solanum tuberosum) (NCBI accessions BQ513657 and BG098563). This fragment, named POTFRT, is illustrated below. Restriction enzyme sites used for DNA cloning into the potato intragenic T-DNA are shown in bold and the frt-like sequence shown in bold and light grey.

| (SEQ ID NO: 43) | |
|---|---|
| 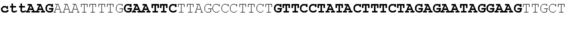 | |
| 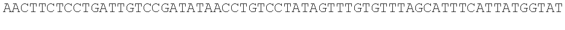 | |
| 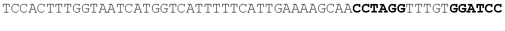 | |
| Nucleotides 1-3 | part of BfrI restriction enzyme site (from |
| | the potato intragenic vector pPOTINV) |
| Nucleotides 4-45 | nucleotides 454 to |
| | 495 of NCBI accession BQ513657 |
| Nucleotides | nucleotides 40 to 179 |
| 46-185 | of NCBI accession BG098563 |

The designed potato frt-like sequence has 5 nucleotide mismatches from the native sequence as illustrated in bold below.

| | |
|---|---|
| | (SEQ ID NO: 44) |
| frt sequence | GAAGTTCCTATACTTTCTAGAGAATAGGAACTTC |
| | (SEQ ID NO: 45) |
| Potato frt-like sequence | 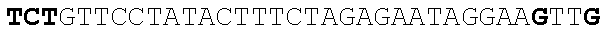 |

The 185 bp POTFRT sequence illustrated above was synthesised by Genscript Corporation (Piscatawa, N.J., www.genscript.com) and supplied cloned into pUC57. All plasmid constructions were performed using standard molecular biology techniques of plasmid isolation, restriction, ligation and transformation into Escherichia coli strain DH5α (Sambrook et al., Molecular Cloning: A Laboratory Manual, 2nd Ed. Cold Spring Harbor Press, 1987).

POTFRT was cloned into the T-DNA composed of potato DNA residing in the plasmid pGEMTPOTINV twice, firstly as a EcoRI to AvrII fragment, then subsequently as a BfrI to BamHI fragment. Confirmation of the POTFRT inserts was verified using restriction enzyme analysis and DNA sequencing. The resulting plasmid was named pPOTFRT2.

The DNA sequence of the 1432 bp SalI fragment comprising the potato derived T-DNA region in the resulting pPOTFRT2 is illustrated below. Only the nucleotides in italics are not part of potato genome sequences. The POTFRT regions are shaded. The T-DNA borders are shown in bold, with the left border positioned at 314-337 and the right border positioned at 1121-1144. Restriction sites illustrated in bold represent those used to clone the POTFRT regions into pGEMTPOTINV. Unique restriction sites in pPOTFRT2 for cloning between POTFRT sites are:

| | |
|---|---|
| AgeI | A/CCGGT |
| BstD102I | GAG/CGG |
| ClaI | AT/CGAT |
| CspI | CG/GWCCG |
| PinAI | A/CCGGT |

(SEQ ID NO: 46)
GTCGACAGTAAAAGTTGCACCTGGAATAAGGTTTTCATTCTTCACAGGAGGCATCTCACTCTTT
CTAGCAGGTCTTGAACGCTTAGATTGAACAGATGTAGGACTCACATCTGATATGGAGGATTCTT
GACTTGTTTCAGCAGCATCAGATGAAGCTTCTGAGACTTCACCTGATCCATCATCTGTAGCAGT
TGCTTCTACTTCTTCCACTGCTACATCAGTCTCAGTTGCTGATACTATAAGACCTCTTAATTTA
GGTCGTAAAATGCAACCAACTCTAAAATGGGGAAACAATTTAATAGATGTTGACAGAGGCAGGA

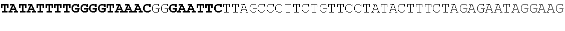

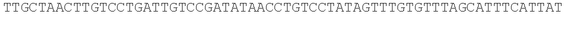

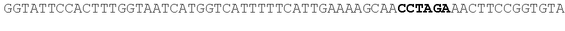

TCCGCCGTTTCCGGCGTTGCACCTCCGCCGAATCTAAAAGGTGCGTTGACGATCATCGATGAGC
GGACCGGTAAGAAGTATCCGGTTCAGGTTTCTGAGGATGGCACTATCAAAGCCACCGACTTAAA

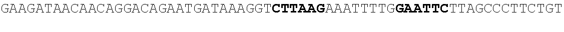

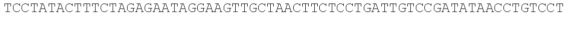

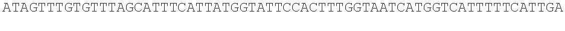

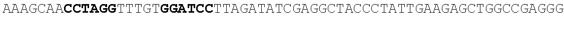

AAGTTCCTTCTTGGAAGTGGCATATCTTTTGTTGTATGGTAATTTACCATCTGAGAACCAGTTA
GCAGACTGGGAGTTCACAGTTTCACAGCATTCAGCGGTTCCACAAGGACTCTTGGATATCATAC
AGTCAATGCCCCATGATGCTCATCCAATGGGGGTTCTTGTCAGTGCAATGAGTGCTCTTTCCGT
TTTTCATCCTGATGCAAATCCAGCTCTGAGAGGACAGGATATATACAAGTGTAAACAATTTAAA
AGCATATGGTGGCACTGCTCAATATATGAGGTGGGCGCGAGAAGCAGGTACCAATGTGTCCTCA
TCAAGAGATGCATTCTTTACCAATCCAACGGTCAAAGCATACTACAAGTCTTTTGTCAAGGCTA
TTGTGACAAGAAAAAACTCTATAAGTGGAGTTAAATATTCAGAAGAGCCCGCCATATTTGCGTG
GGAACTCATAAATGAGCCTCGTTGTGAATCCAGTTCATCAGCTGCTGCTCTCCAGGCGTGGATA
GCAGAGATGGCTGGATTTGTCGAC

The ability of this construct to undergo recombination between the POTFRT sites was tested in vivo using FLP recombinase expressing Escherichia coli strain 294-FLP (Buchholz et al., 1996, Nucleic Acids Research 24 (15) 3118-3119). The binary vector pPOTFRT2 was transformed into E. coli strain 294-FLP and maintained by selection with 100 mg/l ampillicin and incubation at 23° C. Raising the temperature to 37° C. induces expression of FLP recombinase in E. coli strain 294-FLP, which effected recombination between the two POTFRT sites in pPOTFRT2. This was evident by a reduction in the size of pPOTFRT2 from 4432 bp to 4086 pb. Plasmid isolated from colonies of E. coli strain 294-FLP transformed with pPOTFRT2 and cultured at 37° C., was restricted with SalI. All colonies tested produced the fragments of 3.0 kb, 1.4 kb, and 1.1 kb. These three fragments represent the pGEMT backbone, the unrecombined POTFRT2 fragment, and the expected fragment from recombination between the POTLOXP sites, respectively.

Recombination between the POTFRT sites was further verified by DNA sequencing. The resulting sequence is illustrated below and confirms that recombination is base pair faithful through the remaining POTFRT site. The remaining POTFRT region is shaded. The left T-DNA border is illustrated in bold and positioned at 253-276. Restriction sites illustrated in bold represent those remaining from cloning the POTFRT regions into pGEMTPOTINV.

(SEQ ID NO: 47)
TTTCTAGCAAGTCTTGTACGCTTAGATTGAACAGATGTAGGACTCACATCTGATATGGAGGATT
CTTGACTTGTTTCAGCAGCATCAGATGAAGCTTCTGAGACTTCACCTGATCCATCATCTGTAGC
AGTTGCTTCTACTTCTTCCACTGCTACATCAGTCTCAGTTGCTGATACTATAAGACCTCTTAAT
TTAGGTCGTAAAATGCAACCAACTCTAAAATGGGGAAACAATTTAATAGATGTTGACAGAGGCA

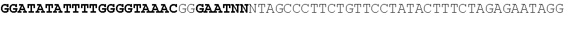

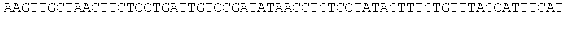

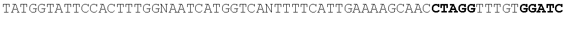

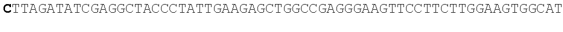

ATCTTTTGTTGTATGGTAATTTACCATCTGAGAACCAGTTAGCAGACTGGGAGTTCACAGTTTC
ACAGCATTCAGCGGTTCCACAAGGACTCTTGGATATCATACAGTCAATGCCCCATGATGCTCAT
CCAATGGGGGTACTTGTCAGTGCAATGAGTGCTCTTTCCGTTTTT

2) Onion (Allium cepa) Frt-Like Fragment—AllFrt

A fragment containing a frt-like sequence was designed from two EST sequences from onion (NCBI accessions CF434781 and CF445353). This fragment, named ALLFRT, is illustrated below. Restriction enzyme sites to allow cloning into the onion intragenic binary vector described in Example 8 are shown in bold and the frt-like sequence is illustrated in bold and light grey.

| (SEQ ID NO: 48) | |
|---|---|
| 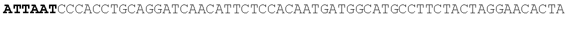 | |
| 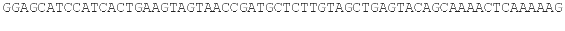 | |
| 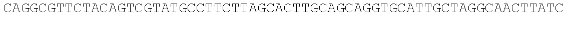 | |
| 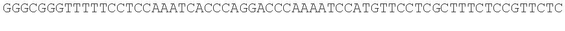 | |
| 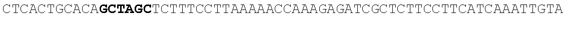 | |
| 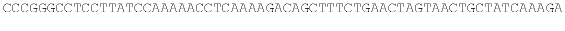 | |
| 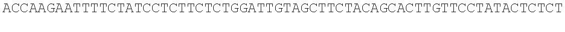 | |
| 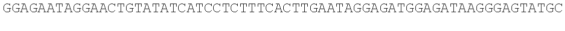 | |
| 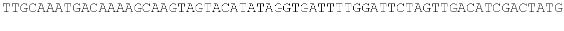 | |
| 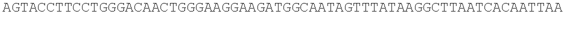 | |
| 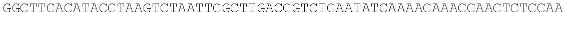 | |
| 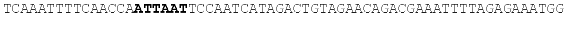 | |
| 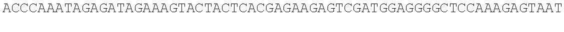 | |
| 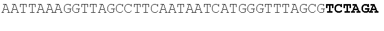 | |
| Nucleotides 1-450 | nucleotides 28-477 of |
| | NCBI accession CF434718 |
| Nucleotides 451-875 | nucleotides 105-529 of NCBI |
| | accession CF445383 |

The designed onion rt-like sequence has 7 nucleotide mismatches from the native frt sequence as illustrated in bold below.

| | |
|---|---|
| | (SEQ ID NO: 49) |
| Frt sequence | GAAGTTCCTATACTTTCTAGAGAATAGGAACTTC |
| | (SEQ ID NO: 50) |
| Onion frt-like sequence | 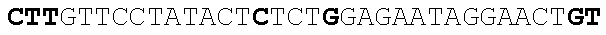 |

The 875 bp ALLFRT sequence can be cloned into pALLINV twice, once via flanking VspI sites into NdeI site of pALLINV and subsequently via NheI and XbaI site into the XbaI site of pALLINV. The correct orientation and confirmation of the ALLFRT insert can be verified by restriction enzyme analysis and DNA sequencing.

The DNA sequence of the 2896 bp SalI fragment comprising the onion derived T-DNA region in the resulting pALLFRT2 is illustrated below. Only the nucleotides in italics are not part of onion genome sequences. The ALLFRT regions are shaded. The T-DNA borders are shown in bold, with the left border positioned at 520-543 and the right border positioned at 2490-2513. Restriction sites illustrated in bold represent those used to clone the ALLFRT regions into the onion T-DNA like sequence.

(SEQ ID NO: 51)
GTCGACTTCCCTTTCCTCTACTCCACTTGTTTCTCGCTTTCTCTACTTCCTTTTTCTCTCTTTT
CTTTATATTTATTGCTCAGCTGGGATTAATTACTGTCATTTATTCCTCATATCTATTTTATTGA
ATTAAAACGGTTATTTAGCTCGAGGCCTTCTCTCTTATTCTTTGCTTCCAAGGAGAGAGAATAT
GGCGAGTGGTAGCAATCATCAGCATGGTGGAGGAGGAAGAAGAAGAGGCGGAATGTTAGTCGCT
GCGACCTTGCTTATTCTTCCTGCCATTTTCCCCAATTTGTTTGTTCCTCTTCCCTTTGCTTTTG
GTAGTTCTGGCAGCGGTGCATCTCCTTCTCTCTTCTCCGAATGGAATGCTCCTAAACCTAGGCA
TCTCTCTCTTCTGAAAGCAGCCATTGAGCGTGAGATTTCTGACGAACAAAAATCAGAGCTGTGG
TCTCCCTTGCCTCCACAGGGATGGAAACCGTGCCTTGAGACTCAATATAGTAGCGGGCTACCCA
GTAGATCGACAGGATATATTCAAGTGTAAAACAAGATGCTGAATCGATTAGCAATGGTTCGCTC

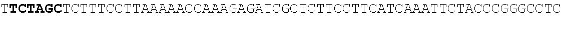

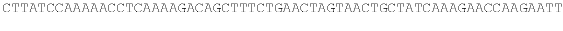

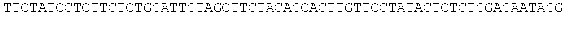

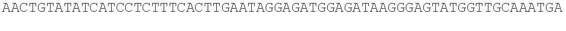

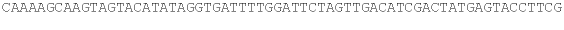

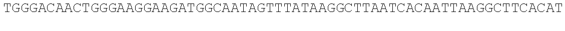

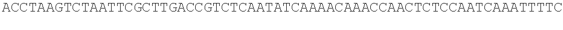

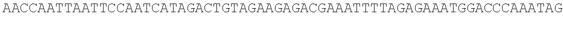

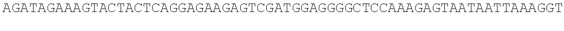

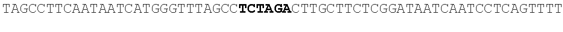

TGATTCCTTCTCGAAGCTTCCTTGATCTCCATAAGATGGTAAACAAGGAGGCGATAAAAAAAGA
AAGGGCTAGACTTGCTGATGAGATGAGCAGAGGATATTTTGCGGATATGGCAGAGATTCGTATA
CATGGTGGCAAGATTGCTATGGCAAATGAAATTCTTATTCCATCAGGGGAAGCAATCAAATTTC
CTGATTTGACAGTAAAATTGTCTGATGATAGCAGTTTGCATTTACCAATTGTATCTACACAAAG
TGCTACAAATAACAATGCTAAATCCACTCCTGCTGCCTCATTGTTGTGCCTTTCCTTCAGAGCA
AGTTCACAGACAATGGTTGAATCATGGACTGTTCCTTTTTTGGACACTTTTAACTCTTCAGAAG

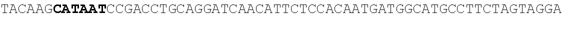

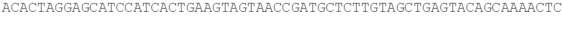

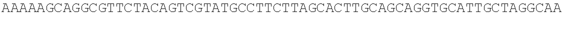

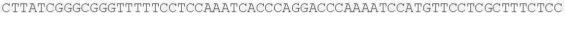

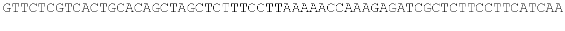

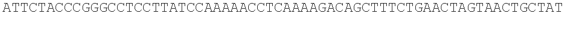

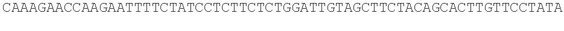

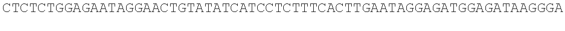

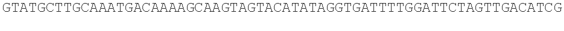

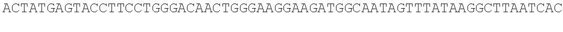

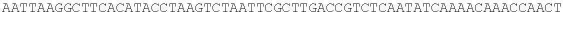

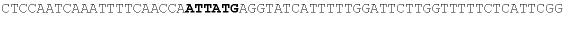

ACCAATCAAGAGAATGTTTCTTAACATGACGAAGAAACCCACTGCTACTCAGCGGAAGATTGGT
TATTTCATTTGGTGATCACTATGATTTTAGGAAGCAGCTTCAAATTGTAAATCTTTTGACAGGA
TATATATTACTGTAAAAAGTGAAGAGAGAAATGTGATATATGCTGATGTTTCCATGGAGAGGGG
TGCATTTCTTGTTCAACAAGCTATGAGGGCTTTCCATGGAAAGAATATAGAAAGCGCAAAATCA
AGGCTTAGTCTTTGCGAGGAGGATATTCGTGGGCAGTTAGAGATGACAGATAACAAACCAGAGT
TATATTCACAGCTTGGTGCTGTCCTTGGAATGCTAGGAGACTGCTGTCGAGGAATGGGTGATAC
TAATGGTGCGATTCCATATTATGAAGAGAGTGTGGAATTCCTCTTAAAAATGCCTGCAAAAGAT
CCCGAGGTTGTACATACACTATCAGTTTCCTTGAATAAAATTGGAGACCTGAAATACTACGAAG
GAGATCTGCAGTCGAC

Restriction enzyme sites available for cloning between ALLFRT sequences include:

| | |
|---|---|
| ApaBI | GCANNNNN/TGC |
| BsiI | C/TCGTG |
| BspMI | ACCTGCNNNN/ |
| DraIII | CACNNN/GTG |
| HindIII | A/AGCTT |
| MfeI | C/AATTG |
| NheI | G/CTAGC |
| PflMI | CCANNNN/NTGG |
| ScaI | AGT/ACT |
| SphI | GCATG/C |
| XbaI | T/CTAGA |

3) Frt-Like Sequences from Other Species
Brassica Napus (Rape) Frt-Like Sequence Designed from 2 ESTs

| | |
|---|---|
| Frt sequence | |
| GAAGTTCCTATACTTTCTAGAGAATAGGAACTTC | (SEQ ID NO: 52) |
| Rape frt-like sequence | |
| ACAGTTCCTATACTTTCTGGAGAATAGGAAGGTG | (SEQ ID NO: 53) |

Nucleotides 1-14
Nucleotides 397-410 of NCBI accession CD824140
Nucleotides 15-34
Nucleotides 128-147 of NCBI accession CD825268

The rape frt-like sequence has 6 nucleotide mismatches from the native frt sequence (illustrated above in bold).

Glycine Max (Soybean) Frt-Like Sequence Designed from 2 ESTs

| | |
|---|---|
| Frt sequence | |
| GAAGTTCCTATACTTTCTAGAGAATAGGAACTTC | (SEQ ID NO: 54) |
| Soybean frt-like sequence | |
| ACAGTTCCTATACTTTCTACAGAATAGGAACTTC | (SEQ ID NO: 55) |

Nucleotides 1-19
Nucleotides 84-102 of NCBI accession BE057270
Nucleotides 20-34
Nucleotides 243-257 of NCBI accession BI970552

The soybean frt-like sequence has 3 nucleotide mismatches from the native frt sequence (illustrated above in bold).

Triticum Aestivum (Wheat) Frt-Like Sequence Designed from 2 ESTs

| | |
|---|---|
| Frt sequence | |
| GAAGTTCCTATACTTTCTAGAGAATAGGAACTTC | (SEQ ID NO: 56) |
| Wheat frt-like sequence | |
| AGAGTTCCTATACTTTCTAGAGAATAGGAACCCC | (SEQ ID NO: 57) |

Nucleotides 1-18
Nucleotides 446-463 of NCBI accession CD877128
Nucleotides 19-34
Nucleotides 1805-1820 of NCBI accession BT009538

The wheat frt-like sequence has 4 nucleotide mismatches from the native frt sequence (illustrated above in bold).

Pinus Taeda (Loblolly Pine) Frt-Like Sequence Designed from 2 ESTs

| | |
|---|---|
| Frt sequence | |
| GAAGTTCCTATACTTTCTAGAGAATAGGAACTTC | (SEQ ID NO: 58) |
| Loblolly pine frt-like sequence | |
| AAAGTTCCTATACTTTCTGGAGAATAGGAAAACA | (SEQ ID NO: 59) |

Nucleotides 1-16
Nucleotides 14-29 of NCBI accession AA556441
Nucleotides 17-34
Nucleotides 764-781 of NCBI accession AF101785

The loblolly pine frt-like sequence has 6 nucleotide mismatches from the native frt sequence (illustrated above in bold).

The above examples illustrate practice of the invention. It will be well understood by skilled in the art that numerous variations and modifications may be made without departing from the spirit and scope of the invention.

### REFERENCES

Anand A, Krichevsky A, Schornack S, Lahaye T, Tang Y, Citovsky V, Mysore K S. Arabidopsis VIRE2 interacting protein2 is required for Agrobacterium T-DNA integration in plants. Plant Cell 2007; 19:1695-1708
Barrell P J, Shang Y J, Cooper P A, Conner A J (2002) Alternative selectable markers for potato transformation using minimal T-DNA vectors. Plant Cell Tissue and Organ Culture 70: 61-8.
Barrell P J, Conner A J (2006) Minimal T-DNA vectors suitable for agricultural deployment of transgenic plants. BioTechniques 41: 708-10.
Bevan M W, Flavell R B, Chilton M D (1983) A chimaeric antibiotic-resistance gene as a selectable marker for plant-cell transformation. Nature 304: 184-97.
Chen Z Y, He C Y, Kay M A (2005) Improved production and purification of minicircle DNA vector free of plasmid bacterial sequences and capable of persistent transgene expression in vivo. Human Gene Therapy, 16: 126-131.
Christey M C (2001) Use of R1 mediated transformation for production of transgenic crop plants. In Vitro Cellular & Developmental Biology-Plant 37: 687-700.
Conner A J (2000) Crops: food, environment and ethics. In: Prebble R, editor. Designer Genes. Dark Horse Publishing Ltd, Wellington. p. 141-51.
Conner A J, Jacobs J M E (2006) GM plants without foreign DNA: implications from new approaches in vector development. In: Roberts A, editor. Proceedings of the 9th International Symposium on the Biosafety of Genetically Modified Organisms: Biosafety research and environmental risk assessment. Jeju Island, Korea. p. 197-203.
Conner A J, Glare T R, Nap J P (2003) The release of genetically modified crops into the environment: II. Overview of ecological risk assessment. The Plant Journal 33: 19-46.
Conner A J, Barrell P J, Jacobs J M E, Baldwin S J, Lokerse A S, Nap J P H (2005) Transformation vectors. International Patent Application Number PCT/NZ2005/000117.
Conner A J, Barrell P J, Baldwin S J, Lokerse A S, Cooper P A, Erasmuson, A K, Nap J P, Jacobs JME (2007) Intragenic vectors for gene transfer without foreign DNA. Euphytica 154: 341-53.
Darquet A M, Cameron B, Wils P, Scherman D, Crouzet J (1997) A new DNA vehicle for nonviral gene delivery: Supercoiled minicircle. Gene Therapy 4: 1341-1349.
Darquet A M, Rangara R, Kreis P, Schwartz B, Naimi S, Delaere P, Crouzet J, Scherman D (1999) Minicircle: An improved DNA molecule for in vitro and in vivo gene transfer. Gene Therapy 6: 209-218.
de Buck S C, de Wilde M, van Montagu A, Depicker (2006) T-DNA vector backbone sequences are frequently integrated into the genome of transgenic plants obtained by Agrobacterium-mediated transformation. Molecular Breeding 6: 459-68.
de Frammond A J, Barton K A, Chilton M D (1983) Mini-Ti: a new vector strategy for plant genetic engineering. Bio/Technology 5: 262-9.
Düring K. (1994) A plant transformation vector with a minimal T-DNA. Transgenic Research 3: 138-140.
Fraley R T, Rogers S G, Horsch R B, Sanders P R, Flick J S, Adams S P, Bittner M L, Brand L A, Fink C L, Fry J S, Galluppi G R, Goldberg S B, Hoffmann N L, Woo S C (1983) Expression of bacterial genes in plant cells. Proceedings of the National Academy of Sciences (USA) 80: 4803-7.
Fraley R T, Rogers S G, Horsch R B, Eichholtz D A, Flick J S, Fink C L, Hoffmann N L, Sanders P R (1985) The SEV system: a new disarmed Ti plasmid vector system for plant transformation. Bio/Technology 3: 629-35.
Gelvin S B (2003) Agrobacterium-mediated plant transformation: the biology behind the “gene-jockeying” tool. Microbiology and Molecular Biology Reviews 67: 16-37.
Gheysen G, Angenon G, van Montagu M (1998) Agrobacterium-mediated plant transformation: a scientifically intriguing story with significant applications. In: Lindsey K. editor. Transgenic Plant Research. Harwood Academic Publishers, Amsterdam; 1998. p. 1-33.
Grant J E, Dommisse E M, Christey M C. Conner A J (1991) Gene transfer to plants using Agrobacterium. In: D. R. Murray (Ed.), Advanced Methods in Plant Breeding and Biotechnology. CAB International, Wallingford. p. 50-73.
Hanson B, Engler D, Moy Y, Newman B, Ralston E, Gutterson N (1999) A simple method to enrich an Agrobacterium-transformed population for plants containing only T-DNA sequences. The Plant Journal 19: 727-34.
Heeres P, Schippers-Rozenboom M, Jacobsen E, Visser R G F (2002) Transformation of a large number of potato varieties: genotype-dependent variation in efficiency and somaclonal variability. Euphytica 124: 13-22.
Hellens R, Mullineaux P, Klee H (2000) A guide to Agrobacterium binary Ti vectors. Trends in Plant Science 5: 446-51.
Herrera-Estrella L, de Block M, Messens E, Hernalsteens J P, van Montagu M, Schell J (1983) Chimeric genes as dominant selectable markers in plant cells. The EMBO Journal 2: 987-95.
Hoekema A, Hirsh P R, Hooykaas P J J, Schilperoort R A (1983) A binary plant vector strategy based on separation of vir- and T-region of the Agrobacterium lumefaciens Ti plasmid. Nature 303: 179-80.
Houba-Herin N, Domin M, Pedron J (1994) Transposition of a Ds element from a plasmid into the plant genome in Nicotiana plumbaginifolia protoplast-derived cells. The Plant Journal 6: 55-66.
Kononov M E, Bassuner B, Gelvin S B (1997) Integration of T-DNA binary vector “backbone” sequences into the tobacco genome: evidence for multiple complex patterns of integration. The Plant Journal 11: 945-57.
Kuraya Y, Ohta S, Fukuda M, Hiei Y, Murai N, Hamada K, Ueki J, Imaseki H, Komari T (2004) Suppression of the transfer of non-T-DNA ‘vector backbone’ sequences by multiple left border repeats in vectors for transformation of higher plants mediated by Agrobaeterium tumefaciens. Molecular Breeding 14: 309-20.
Nap J P, Metz P L J, Escaler M, Conner A J (2003) The release of genetically modified crops into the environment: I. Overview of current status and regulations. The Plant Journal 33: 1-18.
Petolino J (2002) Direct DNA delivery into intact cells and tissues. In: Khachatourians G G, McHughen A, Scorza R, Nip W K, Hui Y H. editors. Transgenic Plants and Crops, Marcel Dekker, Inc, New York. p. 137-43.
Porsch P, Jahnke A, and During K (1998) A plant transformation vector with a minimal T-DNA II. Irregular integration patterns of the T-DNA in the plant genome. Plant Molecular Biology 37: 581-585.
Rommens C M, Humara J M, Ye J S, Yan H, Richael C, Zhang L, Perry R, Swords K (2004) Crop improvement through modification of the plant's own DNA. Plant Physiology 135: 421-31.
Stougaard J (1993) Substrate-dependent negative selection in plants using a bacterial cytosine deaminase gene. The Plant Journal 3: 755-61.
Sugimoto K, Otsuki Y, Saji S, Hirochika H (1994) Transposition of the maize Ds element from a viral vector to the rice genome. The Plant Journal 5: 863-871.
Twyman R M, Christou P (2004) Plant transformation technology: particle bombardment. In: Christou P, Klee H. editors. Handbook of Plant Biotechnology. John Wiley & Son, New York. p. 263-89.
Uze M, Potrykus I, Sautter C (1999) Single-stranded DNA in the genetic transformation of wheat (Triticum aestivum L.): transformation efficiency and integration pattern. Theoretical and Applied Genetics 99: 487-95.
Wenck A, Czako M, Kanevski I, Marton L (1997) Frequent collinear long transfer of DNA inclusive of the whole binary vector during Agrobacterium-mediated transformation. Plant Molecular Biology 34: 913-922.
Yan H, Rommens C M 2007. Transposition-based plant transformation. Plant Physiology 143: 570-578.
Yang A, Su Q, An L 2008. Ovary-drip transformation: a simple method for directly generating vector- and marker-free transgenic maize (Zea mays L.) with a linear GFP cassette transformation. Planta in press; DOI 10.1007/s00425-008-0871-5.
Zambryski P, Joos H, Genetello C, Leemans J, van Montagu M, Schell J (1983) Ti plasmid vector for the introduction of DNA into plant cells without alteration of their normal regeneration capacity. EMBO Journal 2: 2143-50.

### SUMMARY OF SEQUENCE LISTING

| SEQ | Sequence | Species/ | | | |
|---|---|---|---|---|---|
| ID NO: | type | Artificial | Molecule | Type | Reference |
| 1 | polynucleotide | artificial | vector | | pUC57PhMCcab |
| 2 | polynucleotide | artificial | | minicircle | Deep Purple |
| 3 | polynucleotide | artificial | | minicircle | Purple Haze |
| 4 | polynucleotide | artificial | vector | | pUC57StMCpatStan2 |
| 5 | polynucleotide | artificial | | minicircle | PatStan2 |
| 6 | polynucleotide | artificial | | expression | Stan2GBSS |
| | | | | cassette | |
| 7 | polynucleotide | artificial | | expression | Stan2Patatin |
| | | | | cassette | |
| 3 | polynucleotide | artificial | vector | | pPOTLOXP2: |
| | | | | | Stan2GBSSPT |
| 9 | polynucleotide | artificial | vector | | pPOTLOXP2: |
| | | | | | Stan2Patatin |
| 10 | polynucleotide | artificial | | minicircle | Stan2GBSSMC |
| 11 | polynucleotide | artificial | | minicircle | Stan2PatatinMC |
| 12 | polynucleotide | artificial | | minicircle | |
| | | | | forming T- | |
| | | | | DNA region | |
| 13 | polynucleotide | artificial | primer | | Cre For |
| 14 | polynucleotide | artificial | primer | | Cre Rev |
| 15 | polynucleotide | artificial | vector | | pMOA38 |
| 16 | polynucleotide | artificial | | minicircle | MOA38MC |
| 17 | polynucleotide | artificial | primer | | LOXPMCF2 |
| 18 | polynucleotide | artificial | primer | | LOXPMCR2 |
| 19 | polynucleotide | artificial | primer | | Cre For New |
| 20 | polynucleotide | artificial | primer | | Cre Rev New |
| 21 | polynucleotide | artificial | vector | | pMOA40 |
| 22 | polynucleotide | artificial | vector | minicircle | MOA40MC |
| 23 | polynucleotide | artificial | primer | | LOXPMCF1 |
| 24 | polynucleotide | artificial | primer | | LOXPMCR1 |
| 25 | polynucleotide | artificial | primer | | LOXPMCF2 |
| 26 | polynucleotide | artificial | primer | | Cre For |
| 27 | polynucleotide | artificial | primer | | Cre Rev |
| 28 | polynucleotide | artificial | | vector insert | potato derived T-DNA |
| | | | | | region |
| 29 | polynucleotide | artificial | | minicircle | POTIV10 |
| 30 | polynucleotide | artificial | | vector insert | potato derived T-DNA |
| | | | | | region |
| 31 | polynucleotide | artificial | | minicircle | POTIV11 |
| 32 | polynucleotide | artificial | | vector insert | POTLOXP |
| 33 | polynucleotide | artificial | | | |
| 34 | polynucleotide | artificial | | | |
| 35 | polynucleotide | artificial | | vector insert | |
| 36 | polynucleotide | artificial | | | |
| 37 | polynucleotide | artificial | | | |
| 38 | polynucleotide | artificial | | | |
| 39 | polynucleotide | artificial | | | |
| 40 | polynucleotide | artificial | | | |
| 41 | polynucleotide | artificial | | | |
| 42 | polynucleotide | artificial | | | |
| 43 | polynucleotide | artificial | | | |
| 44 | polynucleotide | artificial | | | |
| 45 | polynucleotide | artificial | | | |
| 46 | polynucleotide | artificial | | | |
| 47 | polynucleotide | artificial | | | |
| 48 | polynucleotide | artificial | | | |
| 49 | polynucleotide | artificial | | | |
| 50 | polynucleotide | artificial | | | |
| 51 | polynucleotide | artificial | | | |
| 52 | polynucleotide | artificial | | | |
| 53 | polynucleotide | artificial | | | |
| 54 | polynucleotide | artificial | | | |
| 55 | polynucleotide | artificial | | | |
| 56 | polynucleotide | artificial | | | |
| 57 | polynucleotide | artificial | | | |
| 58 | polynucleotide | artificial | | | |
| 59 | polynucleotide | artificial | | | |
| 60 | polynucleotide | artificial | primer | | NA34For |
| 61 | polynucleotide | artificial | primer | | PETCABPTRev |
| 62 | polynucleotide | artificial | primer | | PanfrtFor |
| 63 | polynucleotide | artificial | primer | | GBSSTermRev |
| 64 | polynucleotide | artificial | | | loxP consensus motif |
| 65 | polynucleotide | artificial | | | fit consensus motif |
| 66 | polynucleotide | artificial | | | T-DNA border-like |
| | | | | | sequence consensus |
| | | | | | motif |
| 67 | polynucleotide | Petunia | | | Petunia Cab 22R |
| | | hybrida | | | promoter |
| 68 | polypeptide | Petunia | | | Petunia Purple Haze |
| | | hybrida | | | |
| 69 | polypeptide | Petunia | | | Petunia Deep Purple |
| | | hybrida | | | |

1-74. (canceled)

75. A vector comprising first and second recombinase recognition sequences,
wherein the vector comprises at least one T-DNA border-like sequence between the recombinase recognition sequences, and wherein the recombinase recognition sequences, and any sequence between the recombinase recognition sequences, are derived from plant species.

76. The vector of claim 75 that comprises two T-DNA border-like sequences between the recombinase recognition sequences,

77. The vector of claim 75 in which the T-DNA border-like sequence or sequences is/are derived from a species interfertile with the plant species from which the recombinase recognition sequences are derived.

78. The vector of claim 75 that is capable of producing a minicircle DNA molecule in the presence of a suitable recombinase.

79. The vector of claim 75 in which the minicircle produced is composed entirely of plant-derived sequence.

80. The vector of claim 75 comprising at least one expression construct between the recombinase recognition sequences.

81. The vector of claim 80 in which the expression construct, and the elements within it, are derived from plants.

82. A vector comprising first and second recombinase recognition sequences, comprising at least one T-DNA border sequence between the recombinase recognition sequences.

83. The vector of claim 82, which is capable of producing a minicircle DNA molecule in the presence of a suitable recombinase.

84. The vector of claim 82 which further comprises at least one expression construct between the recombinase recognition sequences.

85. The vector of claim 82 which comprises, between the recombinase recognition sequences, at least one T-DNA border-like sequence, in place of the T-DNA border sequence.

86. A minicircle DNA molecule composed entirely of sequences derived from plant species, generated from a vector comprising first and second recombinase recognition sequences wherein the recombinase recognition sequences, and any sequence between the recombinase recognition sequences, are derived from plant species.

87. A minicircle DNA molecule of claim 86 that is generated from a vector comprising at least one T-DNA-like border sequence between the recombinase recognition sequences.

88. The minicircle DNA molecule of claim 86 comprising at least one expression construct.

89. The minicircle DNA molecule of claim 88, wherein the expression construct includes a light-regulated promoter.

90. The minicircle DNA molecule of claim 88, wherein the expression construct includes a sequence to be expressed encoding a polypeptide that is an R2R3 MYB transcription factor.

91. The minicircle DNA molecule of claim 86 which comprises at least one T-DNA border-like sequence.

92. A minicircle DNA molecule comprising at least one T-DNA border sequence.

93. A minicircle DNA molecule comprising at least one T-DNA border sequence that is generated from the vector of claim 8.

94. The minicircle of claim 92, that comprises at least one expression construct.

95. The minicircle DNA molecule of claim 94, wherein the expression construct includes a light-regulated promoter.

96. The minicircle DNA molecule of claim 94, wherein the expression construct includes a sequence to be expressed encoding a polypeptide that is an R2R3 MYB transcription factor.

97. The minicircle of claim 92, wherein said minicircle comprises at least one T-DNA border-like sequence, in place of the T-DNA border sequence.

98. A plant cell or plant or plant tissue, organ, propagule or progeny of the plant transformed with a minicircle of claim 86.

99. A plant cell or plant or plant tissue, organ, propagule or progeny of the plant transformed with a minicircle of claim 92.

100. A method for producing a minicircle, the method comprising contacting a vector of claim 75 with a recombinase, to produce a minicircle by site-specific recombination.

101. A method for producing a minicircle, the method comprising contacting a vector of claim 82 with a recombinase, to produce a minicircle by site-specific recombination.

102. A method for transforming a plant, the method comprising introducing a minicircle DNA molecule into a plant cell, or plant, to be transformed, wherein the minicircle DNA molecule is a minicircle DNA molecule of claim 86.

103. A method for transforming a plant, the method comprising introducing a minicircle DNA molecule into a plant cell, or plant, to be transformed, wherein the minicircle DNA molecule is a minicircle DNA molecule of claim 92.

104. The method of claim 102 comprising the additional step of generating the minicircle DNA molecule from a vector, prior to introducing the minicircle into the plant.

105. The method of claim 103 comprising the additional step of generating the minicircle DNA molecule from a vector, prior to introducing the minicircle into the plant.

106. A plant cell or plant produced by a method of claim 102.

107. A plant cell or plant produced by a method of claim 103.
